(19) United States
(12) Patent Application Publication
(10) Pub. No.: US 2023/0141433 A1
(43) Pub. Date: May 11, 2023

(54) TRICYCLIC PESTICIDAL COMPOUNDS

(71) Applicant: BASF SE, Ludwigshafen (DE)

(72) Inventors: Rizwan Shabbir Shaikh, Navi Mumbai (IN); Wolfgang von Deyn, Neustadt (DE); Pulakesh Maity, Navi Mumbai (IN); Birte Schroeder, Ludwigshafen (DE); Rupsha Chaudhuri, Navi Mumbai (IN); Sunderraman Sambasivan, Navi Mumbai (IN); Ashokkumar Adisechan, Navi Mumbai (IN)

(21) Appl. No.: 17/918,135

(22) PCT Filed: Mar. 31, 2021
(86) PCT No.: PCT/EP2021/058526

§ 371 Date: Oct. 11, 2022

(30) Foreign Application Priority Data
Apr. 14, 2020 (IN) 202021016140
May 27, 2020 (EP) 20176691.2

(51) Int. Cl. C07D 519/00; A01N 43/90; A01P 7/04

(52) U.S. Cl. CPC C07D 519/00; A01N 43/90; A01P 7/04

(57) ABSTRACT

The invention relates to compounds of formula (I), wherein the variables are as defined in the specification. It also relates to the use of compounds of formula (I) as an agrochemical pesticide; to pesticidal mixtures comprising compounds of formula (I); and to agrochemical or veterinary compositions comprising compounds of formula (I). Other objects are seed comprising compounds of formula (I); and methods for controlling invertebrate pests, infestation, or infection by invertebrate pests by application of compounds of formula (I).

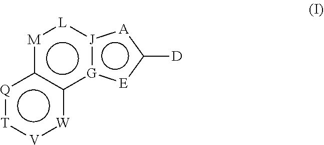

The invention relates to compounds of formula (I) or an agrochemically or veterinarily acceptable salt, stereoisomer, tautomer, or N-oxide thereof

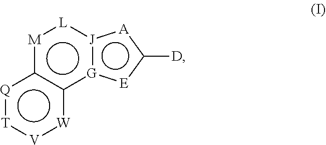

wherein the variables are as defined below. The invention also relates to the use of compounds of formula (I) as an agrochemical pesticide; to pesticidal mixtures comprising a compound of formula (I) and another agrochemically active ingredient; to agrochemical or veterinary compositions comprising a compound of formula (I) or the pesticidal mixture and a liquid or solid carrier; and to seed comprising a compound of formula (I) or the pesticidal mixture. The invention also relates to methods for controlling invertebrate pests, infestation, or infection by invertebrate pests by application of the compounds of formula (I) or the pesticidal mixtures comprising them.

Invertebrate pests and in particular insects, arachnids and nematodes destroy growing and harvested crops and attack wooden dwelling and commercial structures, thereby causing large economic loss to the food supply and to property. Accordingly, there is an ongoing need for new agents for combating invertebrate pests.

WO2017/167832A1 discloses bicyclic compounds and their use as agrochemical pesticides, whereas tricyclic compounds are not described.

Due to the ability of target pests to develop resistance to pesticidally active agents, there is an ongoing need to identify further compounds, which are suitable for combating invertebrate pests such as insects, arachnids and nematodes. Furthermore, there is a need for new compounds having a high pesticidal activity and showing a broad activity spectrum against a large number of different invertebrate pests, especially against difficult to control insects, arachnids and nematodes. There is furthermore a need to find compounds that display a higher efficacy as compared with known pesticides, which reduces the application rates and costs for the applicant, and decreases the environmental effects on soil and ground water.

It is therefore an object of the present invention to identify and provide compounds, which exhibit a high pesticidal activity and have a broad activity spectrum against invertebrate pests.

It has been found that these objects can be achieved by substituted tricyclic compounds of formula I as depicted and defined below, including their stereoisomers, their salts, in particular their agriculturally or veterinarily acceptable salts, their tautomers and their N-oxides.

Therefore, the invention provides in a first aspect compounds of formula (I), or an agrochemically or veterinarily acceptable salt, stereoisomer, tautomer, or N-oxide thereof

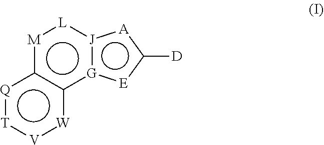

wherein the variables in formula (I) have the following meaning,
A is CH, N, or NH;
E is N, O, S, NR^{E}, or OR^{E};
G, J are independently C or N;
L is N or CR^{L};
M is N or CR^{M};
Q is N or CR^{Q};
T is N or CR^{T};
V is N or CR^{V};
W is N or CR^{W};
R^{E}, R^{L}, R^{M}, R^{Q}, R^{T}, R^{V}, and R^{W} are independently selected from H, halogen, N₃, CN, NO₂, SCN, SF₅, C₁-C₆-alkyl, C₁-C₆-alkoxy, C₂-C₆-alkenyl, tri-C₁-C₆-alkylsilyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxyx-C₁-C₄-alkyl, which groups are unsubstituted or substituted with halogen;
   C(═O)OR¹, NR²R³, C₁-C₆-alkylen-NR²R³, O—C₁-C₆-alkylen-NR²R³, C₁-C₆-alkylen-CN, NH—C₁-C₆-alkylen-NR²R³, C(═O)NR²R³, C(═O)R⁴, SO₂NR²R³, S(═O)_{q}R⁵, OR⁶, SR⁶, phenyl, and benzyl, wherein the phenyl ring g is unsubstituted or substituted with one or more, same or different substituents R¹¹;
   R¹ is H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, or C₃-C₆-cycloalkoxy-C₁-C₄-alkyl, which groups are unsubstituted or substituted with halogen;
      C₁-C₆-alkylen-NR²R³, C₁-C₆-alkylen-CN, or
      phenyl or benzyl, wherein the phenyl ring is unsubstituted, or substituted with one or more, same or different substituents R¹¹;
      R¹¹ is selected from halogen, N₃, OH, CN, NO₂, SCN, SF₅, C₁-C₆-alkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, which groups are unsubstituted or substituted with halogen;
   R² is H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted, or substituted with one or more, same or different substituent selected from halogen, CN and HO;
      C(═O)R²¹, C(═O)OR²¹, C(═O)NR²¹, C₁-C₆-alkylen-CN, or phenyl or benzyl, wherein the phenyl ring is unsubstituted or substituted with one or more, same or different substituents R¹¹;
      R²¹ is H, C₁-C₆-alkyl, C₁-C₆-haloalkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄ alkyl, phenyl, or a saturated, partially-, or fully unsaturated 5- or 6-membered heterocycle, wherein the cyclic moieties are unsubstituted or substituted with one or more, same or different substituents R¹¹;
   R³ is H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen; C₁-C₆-alkylen-CN, or phenyl or benzyl, wherein the phenyl ring is unsubstituted or substituted with one or more, same or different substituents R¹¹; or
   NR²R³ may also form an N-bound, saturated 3- to 8-membered heterocycle, which in addition to the nitrogen atom may have 1 or 2 further heteroatoms or heteroatom moieties selected from O, S(═O)_{q}, NH, and N—C₁-C₆-alkyl, and wherein the N-bound heterocycle is unsubstituted or substituted with one or more, same or different substituents selected from halogen, C₁-C₄-alkyl, C₁-C₄-haloalkyl, C₁-C₄-alkoxy and C₁-C₄-haloalkoxy;
   R⁴ is H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, or C₃-C₆-cycloalkoxy-C₁-C₄-alkyl, which groups are unsubstituted or substituted with one or more, same of different substituents selected from halogen, CN, and OH; phenyl or benzyl, wherein the phenyl ring unsubstituted, or substituted with one or more, same or different substituents R¹¹;
   R⁵ is C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, or C₃-C₆-cycloalkoxy-C₁-C₄-alkyl, which groups are unsubstituted or substituted with halogen; C₁-C₆-alkylen-NR²R³, C₁-C₆-alkylen-CN, phenyl or benzyl, wherein the phenyl ring is unsubstituted, or substituted with one or more, same or different substituents R¹¹;
   R⁶ is phenyl, which is unsubstituted or substituted with one or more, same or different substituents R¹¹;
D is a moiety of formula

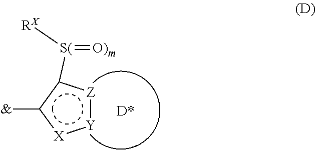

wherein the “&”-symbol signifies the connection to the remainder of formula (I), wherein the dotted circle in the 5-membered ring means that the 5-membered ring may be saturated, partially unsaturated, or fully unsaturated;
R^{X} is C₁-C₆-alkyl, C₃-C₆-cycloalkyl, C₃-C₆-cycloalkyl-C₁-C₄-alkyl, which are unsubstituted or substituted with halogen; or phenyl or benzyl, wherein the phenyl ring is unsubstituted or substituted with one or more, same or different substituents R¹¹;
   X is N, S, O, CR⁷, or NR⁸;
   Y and Z are independently C or N, wherein at least one of the variables selected from Y and Z is C;
   D* is a 5- or 6-membered saturated, partially unsaturated, or fully unsaturated carbo- or heterocycle, which carbo- or heterocycle includes the atoms Y and Z as ring members and is unsubstituted or substituted with one or more, same or different substituents R⁹, and wherein said heterocycle comprises 0, 1, 2, or 3, same or different heteroatoms O, N, or S in addition to those that may be present as ring members Y and Z;
      R⁷ is H, halogen, OH, CN, NC, NO₂, N₃, SCN, NCS, NCO, SF₅, C₁-C₆-alkyl, C₃-C₆-cycloalkyl, C₂-C₆-alkenyl, C₃-C₆-cycloalkenyl, C₂-C₆-alkynyl, which groups are unsubstituted, or substituted with one or more, same or different substituents R^{G1};
         a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents R^{H1}, and wherein said N- and S-atoms are independently oxidized, or non-oxidized;
         phenyl, which is unsubstituted, or substituted with one or more, same or different substituents R^{J1};
         OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═S)SR^{K1}, OS(═O)_{q}R^{K1}, OS(═)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})(═O)OR^{K1}, S(═O)_{q}R^{V1}, SC(═O)SR^{K1}, SC(═O)NR^{L1}R^{M1}, S(═O)_{q}NR^{L1}R^{M1}, C(═O)R^{P1}, C(═S)R^{P1}, C(═O)NR^{L1}R^{M1}, C(═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, Si(R^{S1})₂R^{T1};
      R⁸ is H, CN, C₁-C₆-alkyl, C₃-C₆-cycloalkyl, C₂-C₆-alkenyl, C₃-C₆-cycloalkenyl, C₂-C₆-alkynyl, which groups are unsubstituted or substituted with one or more, same or different substituents R^{G1};
         a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents R^{H1}, and wherein said N- and S-atoms are independently oxidized, or non-oxidized; phenyl, which is unsubstituted, or substituted with one or more, same or different substituents R^{J1};
         OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═S)SR^{K1}, OS(═)R^{K1}, OS(═O)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)_{q}R^{V1}, SC(═O)SR^{K1}, SC(═O)NR^{L1}R^{M1}, S(═O)_{q}NR^{L1}R^{M1}, C(═O)R^{P1}, C(═S)R^{P1}, C(═O)NR^{L1}R^{M1}, C(═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, or Si(R^{S1})₂R^{T1};
      each R⁹ is independently H, halogen, OH, CN, NC, NO₂, N₃, SCN, NCS, NCO, SF₅, C₁-C₆-alkyl, C₃-C₆-cycloalkyl, C₂-C₆-alkenyl, C₃-C₆-cycloalkenyl, or C₂-C₆-alkynyl, C₃-C₆-cycloalkyl-C₁-C₃-alkyl, which groups are unsubstituted, or substituted with one or more, same or different substituents R^{G1};
         a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents R^{H1}, and wherein said N- and S-atoms are independently oxidized, or non-oxidized;
         phenyl, which is unsubstituted, or substituted with one or more, same or different substituents R^{J1};
         OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═S)SR^{K1}, OS(O)_{q}R^{K1}, OS(═O)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)_{q}R^{V1}, SC(═O)SR^{K1}, SC(═O)NR^{L1}R^{M1}, S(═O)_{q}NR^{L1}R^{M1}, C(═O)R^{P1}, C(═S)R^{P1}, C(═O)NR^{L1}R^{M1}, C(═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, or Si(R^{S1})₂R^{T1};
         or two substituents R^{G1} form, together with the ring members of ring D to which they are bound, a 5- or 6-membered saturated, partially unsaturated, or fully unsaturated carbo- or heterocycle, which carbo- or heterocycle is unsubstituted, or substituted with one or more, same or different substituents R^{J1}, and wherein said heterocycle comprises one or more, same or different heteroatoms O, N, or S;
         each R^{G1} is independently halogen, OH, CN, NC, NO₂, C₁-C₆-alkyl, C₃-C₆-cycloalkyl, C₃-C₆-cycloalkenyl, which groups are unsubstituted or substituted with one or more, same or different substituents selected from halogen, OH, CN, C₁-C₃-alkoxy, C₁-C₃-haloalkoxy, and C₁-C₃-alkyl-carbonyl;
            a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents selected from halogen, OH, CN, C₁-C₃-alkoxy, C₁-C₃-haloalkoxy, and C₁-C₃-alkyl-carbonyl, and wherein said N- and S-atoms are independently oxidized, or non-oxidized;
            phenyl, which is unsubstituted or substituted with one or more, same or different substituents selected from halogen, OH, CN, NO₂, C₁-C₃-alkyl, C₁-C₃-haloalkyl, C₁-C₃-alkoxy, C₁-C₃-haloalkoxy, and C₁-C₃-alkyl-carbonyl; OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═S)SR^{K1}, OS(═)R^{K1}, OS(═)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)_{q}R^{V1}, SC(═O)SR^{K1}, SC(═O)NR^{L1}R^{M1}, S(═O)_{q}NR^{L1}R^{M1}, C(═O)R^{P1}, C(═S)R^{P1}, C(═O)NR^{L}R^{M1}, (═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, Si(R^{S1})₂R^{T1};
         each R^{H1} is independently halogen, CN, NC, NO₂, SCN, NCS, NCO, C₁-C₆-alkyl, C₃-C₆-cycloalkyl, C₃-C₆-cycloalkenyl, which groups are unsubstituted, or substituted with one or more, same or different substituents selected from halogen, OH, CN, C₁-C₁₀-alkoxy, C₁-C₃-haloalkoxy, and C₁-C₃-alkyl-carbonyl;
            phenyl, which is unsubstituted, or substituted with one or more, same or different substituents selected from halogen, OH, CN, NO₂, C₁-C₃-alkyl, C₁-C₃-haloalkyl, OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═K)SR^{K1}, OS(═)R^{K1}, OS(═)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)_{q}R^{V1}, SC(═O)SR^{K1}, SC(═O)NR^{L1}R^{M1}, S(═O)_{q}NR^{L1}R^{M1}, (═O)R^{P1}, (═S)R^{P1}, C(═O)NR^{L1}R^{M1}, (═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, Si(R^{S1})₂R^{T1}; or
            two geminal substituents R^{H1} form together with the atom to which they are bound a group ═O, ═S, or ═NR^{L};
         each R^{J1} is independently halogen, CN, NC, NO₂, SCN, NCS, NCO, C₁-C₆-alkyl, C₃-C₆-cycloalkyl, C₃-C₆-cycloalkenyl, which groups are unsubstituted, or substituted with one or more, same or different substituents selected from halogen, OH, CN, C₁-C₁₀-alkoxy, C₁-C₃-haloalkoxy, and C₁-C₃-alkyl-carbonyl;
            phenyl, which is unsubstituted, or substituted with one or more, same or different substituents selected from halogen, OH, CN, NO₂, C₁-C₃-alkyl, C₁-C₃-haloalkyl, OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═S)SR^{K1}, OS(═)R^{K1}, OS(═)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)R^{V1}, SC(═O)SR^{K1}, SC(═O)NR^{L1}R^{M1}, S(═O)_{q}NR^{L1}R^{M1}, C(═O)R^{P1}, C(═S)R^{P1}, C(═O)NR^{L1}R^{M1}, (═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, Si(R^{S1})₂R^{T1};
         each R^{K1} is independently H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with one or more, same or different substituents selected from halogen, CN, NR^{M1}R^{N1};
            C(═O)NR^{M1}R^{N1}, C(═O)R^{T1}; or
            phenyl or benzyl, wherein the phenyl ring is unsubstituted or substituted with one or more, same or different substituents R^{X1};
         each R^{L1} is independently H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen; C₁-C₆-alkylen-CN;
            phenyl and benzyl, which groups are unsubstituted or substituted with one or more, same or different substituents R^{X1};
         each R^{M1}, R^{R1} is independently H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen;
            C₁-C₆-alkylen-CN; or
            phenyl or benzyl, wherein the phenyl ring is unsubstituted or substituted with one or more, same or different substituents R^{X1};
         each moiety NR^{M1}R^{R1} or NR^{L1}R^{M1} may also form an N-bound, saturated 5- to 8-membered heterocycle, which in addition to the nitrogen atom may have 1 or 2 further heteroatoms or heteroatom moieties selected from O, S(═O)_{q}, and N—R′, wherein R′ is H or C₁-C₆-alkyl and wherein the N-bound heterocycle is unsubstituted or substituted with one or more, same or different substituents selected from halogen, C₁-C₄-alkyl, C₁-C₄-haloalkyl, C₁-C₄-alkoxy and C₁-C₄-haloalkoxy;
         each R^{N1} is independently H, halogen, CN, NO₂, SCN, C₁-C₁₀-alkyl, C₃-C₃-cycloalkyl, C₂-C₆-alkenyl, C₃-C₆-cycloalkenyl, C₂-C₆-alkynyl, which groups are unsubstituted, or substituted with one or more, same or different substituents selected from halogen, C₁-C₆-alkyl, C₁-C₆-alkoxy, C₁-C₆-haloalkyl, and C₁-C₆-haloalkoxy;
            a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents selected from halogen, C₁-C₃-alkyl, C₁-C₃-alkoxy, C₁-C₃-haloalkyl, and C₁-C₃-haloalkoxy, and wherein said N- and S-atoms are independently oxidized, or non-oxidized;
            phenyl, which is unsubstituted, or substituted with one or more, same or different substituents selected from halogen, C₁-C₃-alkyl, C₁-C₃-alkoxy, C₁-C₃-haloalkyl, and C₁-C₃-haloalkoxy;
         each R^{O1} is independently H, C₁-C₄-alkyl, C₁-C₆-cycloalkyl, C₁-C₂-alkoxy-C₁-C₂-alkyl, phenyl, or benzyl;
         each R^{P1} is independently H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen;
            phenyl or benzyl, wherein the phenyl ring is unsubstituted or substituted with one or more, same or different substituents R^{X1};
         each R^{S1}, R^{T1} is independently H, C₁-C₆-alkyl, C₁-C₆-haloalkyl, C₁-C₆-alkoxy, C₁-C₄-alkoxy-C₁-C₄-alkyl, C₃-C₆-cycloalkyl, C₃-C₆-halocycloalkyl, C₁-C₄-haloalkoxy-C₁-C₄-alkyl, or phenyl;
         each R^{V1} is independently C₁-C₆-alkyl, C₃-C₆-cycloalkyl, C₃-C₆-cycloalkyl-C₁-C₄-alkyl, which are unsubstituted or substituted with halogen; or phenyl or benzyl, wherein the phenyl ring is unsubstituted or substituted with R^{X1};
         each R^{X1} is independently halogen, N₃, OH, CN, NO₂, SCN, SF₅, C₁-C₆-alkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, which groups are unsubstituted or substituted with halogen;
   the index m is 0, 1, or 2;
   the index q is 0, 1, or 2.

The tricyclic compounds of the formula (I), and their agriculturally acceptable salts are highly active against animal pest, i.e. harmful arthropodes and nematodes, especially against insects and acaridae which are difficult to control by other means.

Moreover, the present invention relates to and includes the following embodiments:
compositions comprising at least one compound of formula (I) as defined above;
agricultural and veterinary compositions comprising an amount of at least one compound of formula (I) or an enantiomer, diasteromer or salt thereof as defined above;
methods for combating invertebrate pests, infestation, or infection by invertebrate pests, which method comprises contacting said pest or its food supply, habitat or breeding grounds with a pesticidally effective amount of at least one compound of formula (I) as defined above or a composition thereof;
methods for controlling invertebrate pests, infestation, or infection by invertebrate pests, which method comprises contacting said pest or its food supply, habitat or breeding grounds with a pesticidally effective amount of at least one compound of formula (I) as defined above or a composition comprising at least one compound of formula (I);
methods for preventing or protecting against invertebrate pests comprising contacting the invertebrate pests, or their food supply, habitat or breeding grounds with compounds of the general formula (I) as defined above or a composition comprising at least one compound of formula (I) as defined above or a composition comprising at least one compound of formula (I);
methods for protecting crops, plants, plant propagation material and/or growing plants from attack or infestation by invertebrate pests comprising contacting or treating the crops, plants, plant propagation material and growing plants, or soil, material, surface, space, area or water in which the crops, plants, plant propagation material is stored or the plant is growing, with a pesticidally effective amount of at least one compound of formula (I) as defined above or a composition comprising at least one compound of formula (I);
non-therapeutic methods for treating animals infested or infected by parasites or preventing animals of getting infected or infested by parasites or protecting animals against infestation or infection by parasites which comprises orally, topically or parenterally administering or applying to the animals a parasiticidally effective amount of a compound of formula (I) as defined above or a composition comprising at least one compound of formula (I);
methods for treating, controlling, preventing or protecting animals against infestation or infection by parasites by administering or applying orally, topically or parenterally to the animals a substituted compound of the general formula (I) as defined above or a composition comprising at least one compound of formula (I);
seed comprising a compound of formula (I) as defined above, in an amount of from 0.1 g to 10 kg per 100 kg of seed;
the use of the compounds of formula (I) as defined above for protecting growing plants or plant propagation material from attack or infestation by invertebrate pests;
the use of compounds of formula (I) or the enantiomers, diastereomers or veterinary acceptable salts thereof for combating parasites in and on animals;
a process for the preparation of a veterinary composition for treating, controlling, preventing or protecting animals against infestation or infection by parasites which comprises adding a parasiticidally effective amount of an compound of formula (I) or the enantiomers, diastereomers and/or veterinary acceptable salt thereof to a carrier composition suitable for veterinary use;
the use of a compound of formula (I) or the enantiomers, diastereomers and/or veterinary acceptable salt thereof for the preparation of a medicament for treating, controlling, preventing or protecting animals against infestation or infection by parasites.

All the compounds of formula (I) and, if applicable, their stereoisomers, their tautomers, their salts or their N-oxides as well as compositions thereof are particularly useful for controlling invertebrate pests, in particular for controlling arthropods and nematodes and especially insects. Therefore, the invention relates to the use of a compound of formula (I) as an agrochemical pesticide, preferably for combating or controlling invertebrate pests, in particular invertebrate pests of the group of insects, arachnids or nematodes.

The term “compound(s) according to the invention” or “compound(s) of formula (I)” as used in the present invention refers to and comprises the compound(s) as defined herein and/or stereoisomer(s), salt(s), tautomer(s) or N-oxide(s) thereof. The term “compound(s) of the present invention” is to be understood as equivalent to the term “compound(s) according to the invention”, therefore also comprising stereoisomer(s), salt(s), tautomer(s) or N-oxide(s) of compounds of formula (I).

The terms “tricyclic scaffold” or “tricyclic moiety” relate to the following moiety of formula (I)

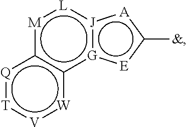

wherein “&” means the remainder of formula (I) and wherein the other variables have a meaning as defined form formula (I). For the avoidance of doubt, it is submitted that the circles in the rings of the tricyclic scaffold above and in any other formula displayed herein means a full unsaturation of the respective ring or ring system, preferably an aromatic ring or ring system.

The term “composition(s) according to the invention” or “composition(s) of the present invention” encompasses composition(s) comprising at least one compound of formula (I) according to the invention as defined above, therefore also including a stereoisomer, an agriculturally or veterinary acceptable salt, tautomer or an N-oxide of the compounds of formula (I).

The compounds of the present invention may be amorphous or may exist in one or more different crystalline states (polymorphs) or modifications which may have a different macroscopic properties such as stability or show different biological properties such as activities. The present invention includes both amorphous and crystalline compounds of the formula (I), mixtures of different crystalline states or modifications of the respective compound of formula (I), as well as amorphous or crystalline salts thereof.

The compounds of the formula (I) may have one or, depending on the substitution pattern, more centers of chirality, in which case they are present as mixtures of enantiomers or diastereomers. The invention provides both the single pure enantiomers or pure diastereomers of the compounds of formula (I), and their mixtures and the use according to the invention of the pure enantiomers or pure diastereomers of the compound of formula (I) or its mixtures. Suitable compounds of the formula (I) also include all possible geometrical stereoisomers (cis/trans isomers) and mixtures thereof. Cis/trans isomers may be present with respect to an alkene, carbon-nitrogen double-bond or amide group. The term “stereoisomer(s)” encompasses both optical isomers, such as enantiomers or diastereomers, the latter existing due to more than one center of chirality in the molecule, as well as geometrical isomers (cis/trans isomers). The present invention relates to every possible stereoisomer of the compounds of formula (I), i.e. to single enantiomers or diastereomers, as well as to mixtures thereof.

Depending on the substitution pattern, the compounds of the formula (I) may be present in the form of their tautomers. Hence the invention also relates to the tautomers of the formula (I) and the stereoisomers, salts, tautomers and N-oxides of said tautomers.

Salts of the compounds of the formula (I) are preferably agriculturally and/or veterinary acceptable salts. They can be formed in a customary method, e.g. by reacting the compound with an acid of the anion in question if the compound of formula (I) has a basic functionality or by reacting an acidic compound of formula (I) with a suitable base.

Suitable agriculturally or veterinary useful salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxy-alkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethyl-ammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formulae I with an acid of the corresponding anion, preferably of hydrochloric acid, hydrobromic acid, sulfuric acid, phosphoric acid or nitric acid.

The term “N-oxide” includes any compound of the present invention which has at least one tertiary nitrogen atom that is oxidized to an N-oxide moiety.

The organic moieties groups mentioned in the above definitions of the variables are—like the term halogen—collective terms for individual listings of the individual group members. The prefix Cₙ-Cₘ indicates in each case the possible number of carbon atoms in the group. “Halogen” will be taken to mean F, Cl, Br, and I, preferably F.

The term “substituted with”, e.g. as used in “partially, or fully substituted with” means that one or more, e.g. 1, 2, 3, 4 or 5 or all of the hydrogen atoms of a given radical have been replaced by one or more, same or different substituents, such as a halogen, in particular F. Accordingly, for substituted cyclic moieties, e.g. 1-cyanocyclopropyl, one or more of the hydrogen atoms of the cyclic moiety may be replaced by one or more, same or different substituents.

The term “Cₙ-Cₘ-alkyl” as used herein (and also in Cₙ-Cₘ-alkylamino, di-Cₙ-Cₘ-alkylamino, Cₙ-Cₘ-alkylaminocarbonyl, di-(Cₙ-Cₘ-alkylamino)carbonyl, Cₙ-Cₘ-alkylthio, Cₙ-Cₘ-alkylsulfinyl and Cₙ-Cₘ-alkylsulfonyl) refers to a branched or unbranched saturated hydrocarbon group having n to m, e.g. 1 to 10 carbon atoms, preferably 1 to 6 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl and their isomers. C₁-C₄-alkyl means for example methyl, ethyl, propyl, 1-methylethyl, butyl, 1-methylpropyl, 2-methylpropyl or 1,1-dimethylethyl.

The term “Cₙ-Cₘ-haloalkyl” as used herein (and also in Cₙ-Cₘ-haloalkylsulfinyl and Cₙ-Cₘ-haloalkylsulfonyl) refers to a straight-chain or branched alkyl group having n to m carbon atoms, e.g. 1 to 10 in particular 1 to 6 carbon atoms (as mentioned above), where some or all of the hydrogen atoms in these groups may be replaced by halogen atoms as mentioned above, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like. The term C₁-C₁₀-haloalkyl in particular comprises C₁-C₂-fluoroalkyl, which is synonym with methyl or ethyl, wherein 1, 2, 3, 4 or 5 hydrogen atoms are substituted with fluorine atoms, such as fluoromethyl, difluoromethyl, trifluoromethyl, 1-fluoroethyl, 2-fluoroethyl, 2,2-difluoroethyl, 2,2,2-trifluoroethyl and pentafluoromethyl.

Similarly, “Cₙ-Cₘ-alkoxy” and “Cₙ-Cₘ-alkylthio” (or Cₙ-Cₘ-alkylsulfenyl, respectively) refer to straight-chain or branched alkyl groups having n to m carbon atoms, e.g. 1 to 10, in particular 1 to 6 or 1 to 4 carbon atoms (as mentioned above) bonded through oxygen (or sulfur linkages, respectively) at any bond in the alkyl group. Examples include C₁-C₄-alkoxy such as methoxy, ethoxy, propoxy, isopropoxy, butoxy, sec-butoxy, isobutoxy and tert-butoxy, further C₁-C₄-alkylthio such as methylthio, ethylthio, propylthio, isopropylthio, and n-butylthio.

Accordingly, the terms “Cₙ-Cₘ-haloalkoxy” and “Cₙ-Cₘ-haloalkylthio” (or Cₙ-Cₘ-haloalkyl-sulfenyl, respectively) refer to straight-chain or branched alkyl groups having n to m carbon atoms, e.g. 1 to 10, in particular 1 to 6 or 1 to 4 carbon atoms (as mentioned above) bonded through oxygen or sulfur linkages, respectively, at any bond in the alkyl group, where some or all of the hydrogen atoms in these groups may be replaced by halogen atoms as mentioned above, for example C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy and pentafluoroethoxy, further C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio and pentafluoroethylthio and the like. Similarly, the terms C₁-C₂-fluoroalkoxy and C₁-C₂-fluoroalkylthio refer to C₁-C₂-fluoroalkyl which is bound to the remainder of the molecule via an oxygen atom or a sulfur atom, respectively.

The term “C₂-Cₘ-alkenyl” as used herein intends a branched or unbranched unsaturated hydrocarbon group having 2 to m, e.g. 2 to 10 or 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “C₂-Cₘ-alkynyl” as used herein refers to a branched or unbranched unsaturated hydrocarbon group having 2 to m, e.g. 2 to 10 or 2 to 6 carbon atoms and containing at least one triple bond, such as ethynyl, propynyl, 1-butynyl, 2-butynyl, and the like.

The term “Cₙ-Cₘ-alkoxy-Cₙ-Cₘ-alkyl” as used herein refers to alkyl having n to m carbon atoms, e.g. like specific examples mentioned above, wherein one hydrogen atom of the alkyl radical is replaced by an Cₙ-Cₘ-alkoxy group; wherein the value of n and m of the alkoxy group are independently chosen from that of the alkyl group.

The suffix “-carbonyl” in a group or “C(═O)” denotes in each case that the group is bound to the remainder of the molecule via a carbonyl C═O group. This is the case e.g. in alkylcarbonyl, haloalkylcarbonyl, aminocarbonyl, alkylaminocarbonyl, dialkylaminocarbonyl, alkoxycarbonyl, haloalkoxycarbonyl.

The term “aryl” as used herein refers to a mono-, bi- or tricyclic aromatic hydrocarbon radical such as phenyl or naphthyl, in particular phenyl (also referred as to C₆H₅ as substituent).

The term “C₃-Cₘ-cycloalkyl” as used herein refers to a monocyclic ring of 3- to m-membered saturated cycloaliphatic radicals, e.g. cyclopropyl, cyclobutyl, cyclopentyl, cyclohexyl, cycloheptyl, cyclooctyl and cyclodecyl.

The term “alkylcycloalkyl” denotes as well as the term “alkyl which may be substituted with cycloalkyl” an alkyl group which is substituted with a cycloalkyl ring, wherein alkyl and cycloakyl are as herein defined.

The term “cycloalkylalkyl” denotes as well as the term “cycloalkyl which may be substituted with alkyl” a cycloalkyl ring which is substituted with an alkyl group, wherein alkyl and cycloakyl are as herein defined.

The term “alkylcycloalkylalkyl” denotes as well as the term “alkylcycloalkyl which may be substituted with alkyl” an alkylcycloalkyl group which is substituted with an alkyl, wherein alkyl and alkylcycloalkyl are as herein defined.

The term “C₃-Cₘ-cycloalkenyl” as used herein refers to a monocyclic ring of 3- to m-membered partially unsaturated cycloaliphatic radicals.

The term “cycloalkylcycloalkyl” denotes as well as the term “cycloalkyl which may be substituted with cycloalkyl” a cycloalkyl substitution on another cycloalkyl ring, wherein each cycloalkyl ring independently has from 3 to 7 carbon atom ring members and the cycloalkyls are linked through one single bond or have one common carbon atom. Examples of cycloalkylcycloalkyl include cyclopropylcyclopropyl (e.g. 1,1′-bicyclopropyl-2-yl), cyclohexylcyclohexyl wherein the two rings are linked through one single common carbon atom (e.g. 1,1′-bicyclohexyl-2-yl), cyclohexylcyclopentyl wherein the two rings are linked through one single bond (e.g. 4-cyclopentylcyclohexyl) and their different stereoisomers such as (1R,2S)-1,1′-bicyclopropyl-2-yl and (1R,2R)-1,1′-bicyclopropyl-2-yl. The term “carbocycle” or “carbocyclyl” includes, unless otherwise indicated, in general a 3- to 12-membered, preferably a 3- to 8-membered or a 5- to 8-membered, more preferably a 5- or 6-membered mono-cyclic, ring comprising 3 to 12, preferably 3 to 8 or 5 to 8, more preferably 5 or 6 carbon atoms.

The carbocyclic radicals may be saturated, partially unsaturated, or fully unsaturated. Preferably, the term “carbocycle” covers cycloalkyl and cycloalkenyl groups as defined above, for example cyclopropane, cyclobutane, cyclopentane and cyclohexane rings. When it is referred to “fully unsaturated” carbocycles, this term also includes “aromatic” carbocycles. In certain preferred embodiments, a fully unsaturated carbocycle is an aromatic carbocycle as defined below, preferably a 6-membered aromatic carbocycle.

The term “hetaryl” or “aromatic heterocycle” or “aromatic heterocyclic ring” includes monocyclic 5- or 6-membered heteroaromatic radicals comprising as ring members 1, 2, 3 or 4 heteroatoms selected from N, O and S. Examples of 5- or 6-membered heteroaromatic radicals include pyridyl, i.e. 2-, 3-, or 4-pyridyl, pyrimidinyl, i.e. 2-, 4- or 5-pyrimidinyl, pyrazinyl, pyridazinyl, i.e. 3- or 4-pyridazinyl, thienyl, i.e. 2- or 3-thienyl, furyl, i.e. 2- or 3-furyl, pyrrolyl, i.e. 2- or 3-pyrrolyl, oxazolyl, i.e. 2-, 3- or 5-oxazolyl, isoxazolyl, i.e. 3-, 4- or 5-isoxazolyl, thiazolyl, i.e. 2-, 3- or 5-thiazolyl, isothiazolyl, i.e. 3-, 4- or 5-isothiazolyl, pyrazolyl, i.e. 1-, 3-, 4- or 5-pyrazolyl, i.e. 1-, 2-, 4- or 5-imidazolyl, oxadiazolyl, e.g. 2- or 5-[1,3,4]oxadiazolyl, 4- or 5-(1,2,3-oxadiazol)yl, 3- or 5-(1,2,4-oxadiazol)yl, 2- or 5-(1,3,4-thiadiazol)yl, thiadiazolyl, e.g. 2- or 5-(1,3,4-thiadiazol)yl, 4- or 5-(1,2,3-thiadiazol)yl, 3- or 5-(1,2,4-thiadiazol)yl, triazolyl, e.g. 1H-, 2H- or 3H-1,2,3-triazol-4-yl, 2H-triazol-3-yl, 1H-, 2H-, or 4H-1,2,4-triazolyl and tetrazolyl, i.e. 1H- or 2H-tetrazolyl. The term “hetaryl” also includes bicyclic 8 to 10-membered heteroaromatic radicals comprising as ring members 1, 2 or 3 heteroatoms selected from N, O and S, wherein a 5- or 6-membered heteroaromatic ring is fused to a phenyl ring or to a 5- or 6-membered heteroaromatic radical. Examples of a 5- or 6-membered heteroaromatic ring fused to a phenyl ring or to a 5- or 6-membered heteroaromatic radical include benzofuranyl, benzothienyl, indolyl, indazolyl, benzimidazolyl, benzoxathiazolyl, benzoxadiazolyl, benzothiadiazolyl, benzoxazinyl, chinolinyl, isochinolinyl, purinyl, 1,8-naphthyridyl, pteridyl, pyrido[3,2-d]pyrimidyl or pyridoimidazolyl and the like. These fused hetaryl radicals may be bonded to the remainder of the molecule via any ring atom of 5- or 6-membered heteroaromatic ring or via a carbon atom of the fused phenyl moiety.

The terms “heterocycle”, “heterocyclyl” or “heterocyclic ring” includes, unless otherwise indicated, in general 3- to 12-membered, preferably 3- to 8-membered, 3- to 7-membered, or 5- to 8-membered, more preferably 5- or 6-membered, in particular 6-membered monocyclic heterocyclic radicals. The heterocyclic radicals may be saturated, partially unsaturated, or fully unsaturated. As used in this context, the term “fully unsaturated” also includes “aromatic”. In a preferred embodiment, a fully unsaturated heterocycle is thus an aromatic heterocycle, preferably a 5- or 6-membered aromatic heterocycle comprising one or more, e.g. 1, 2, 3, or 4, preferably 1, 2, or 3 heteroatoms selected from N, O and S as ring members. Examples of aromatic heterocycles are provided above in connection with the definition of “hetaryl”. Unless otherwise indicated, “hetaryls” are thus covered by the term “heterocycles”. The heterocyclic non-aromatic radicals usually comprise 1, 2, 3, 4 or 5, preferably 1, 2 or 3 heteroatoms selected from N, O and S as ring members, where S-atoms as ring members may be present as S, SO or SO₂. Examples of 5- or 6-membered heterocyclic radicals comprise saturated or unsaturated, non-aromatic heterocyclic rings, such as oxiranyl, oxetanyl, thietanyl, thietanyl-S-oxid (S-oxothietanyl), thietanyl-S-dioxid (S-dioxothiethanyl), pyrrolidinyl, pyrrolinyl, pyrazolinyl, tetrahydrofuranyl, dihydrofuranyl, 1,3-dioxolanyl, thiolanyl, S-oxothiolanyl, S-dioxothiolanyl, dihydrothienyl, S-oxodihydrothienyl, S-dioxodihydrothienyl, oxazolidinyl, oxazolinyl, thiazolinyl, oxathiolanyl, piperidinyl, piperazinyl, pyranyl, dihydropyranyl, tetrahydropyranyl, 1,3- and 1,4-dioxanyl, thiopyranyl, S. oxothiopyranyl, S-dioxothiopyranyl, dihydrothiopyranyl, S-oxodihydrothiopyranyl, S-dioxodihydrothiopyranyl, tetrahydrothiopyranyl, S-oxotetrahydrothiopyranyl, S-dioxotetrahydrothiopyranyl, morpholinyl, thiomorpholinyl, S-oxothiomorpholinyl, S-dioxothiomorpholinyl, thiazinyl and the like. Examples for heterocyclic ring also comprising 1 or 2 carbonyl groups as ring members comprise pyrrolidin-2-onyl, pyrrolidin-2,5-dionyl, imidazolidin-2-onyl, oxazolidin-2-onyl, thiazolidin-2-onyl and the like.

The terms “alkylene”, “alkenylene”, and “alkynylene” refer to alkyl, alkenyl, and alkynyl as defined above, respectively, which are bonded to the remainder of the molecule, via two atoms, preferably via two carbon atoms, of the respective group, so that they represent a linker between two moieties of the molecule. In particular, the term “alkylene” may refer to alkyl chains such as CH₂CH₂, —CH(CH₃)—, CH₂CH₂CH₂, CH(CH₃)CH₂, CH₂CH(CH₃), CH₂CH₂CH₂CH₂, CH₂CH₂CH₂CH₂CH₂, CH₂CH₂CH₂CH₂CH₂CH₂, and CH₂CH₂CH₂CH₂CH₂CH₂CH₂. Similarly, “alkenylene” and “alkynylene” may refer to alkenyl and alkynyl chains, respectively.

The term “5- to 6-membered carbocyclic ring” as used herein refers to cyclopentane and cyclohexane rings.

Examples of 5- or 6-membered saturated heterocyclic rings include: 2-tetrahydrofuranyl, 3-tetrahydrofuranyl, 2-tetrahydrothienyl, 3-tetrahydrothienyl, 2-pyrrolidinyl, 3-pyrrolidinyl, 3-pyrazolidinyl, 4-pyrazolidinyl, 5-pyrazolidinyl, 2-imidazolidinyl, 4-imidazolidinyl, 2-oxazolidinyl, 4-oxazolidinyl, 5-oxazolidinyl, 3-isoxazolidinyl, 4-isoxazolidinyl, 5-isoxazolidinyl, 2-thiazolidinyl, 4-thiazolidinyl, 5-thiazolidinyl, 3-isothiazolidinyl, 4-isothiazolidinyl, 5-isothiazolidinyl, 1,2,4-oxadiazolidin-3-yl, 1,2,4-oxadiazolidin 5 yl, 1,2,4-thiadiazolidin-3-yl, 1,2,4-thiadiazolidin-5-yl, 1,2,4-triazolidin-3-yl,-1,3,4-oxadiazolidin-2-yl, 1,3,4-thiadiazolidin-2-yl, 1,3,4-triazolidin-2-yl, 2-tetrahydropyranyl, 4-tetrahydropyranyl, 1,3-dioxan-5-yl, 1,4-dioxan-2-yl, 2-piperidinyl, 3-piperidinyl, 4-piperidinyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl, 2-morpholinyl, 3-morpholinyl, 2-thiomorpholinyl, 3-thiomorpholinyl, 1-oxothiomorpholin-2-yl, 1-oxothiomorpholin-3-yl, 1,1-dioxothiomorpholin-2-yl, 1,1-dioxothiomorpholin-3-yl.

Examples of 5- or 6-membered partially unsaturated heterocyclyl or heterocyclic rings include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin 3 yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3 dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl.

Examples of 5- or 6-membered fully unsaturated heterocyclic (hetaryl) or heteroaromatic rings are: 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

A “C₂-Cₘ-alkylene” is divalent branched or preferably unbranched saturated aliphatic chain having 2 to m, e.g. 2 to 7 carbon atoms, for example CH₂CH₂, —CH(CH₃)—, CH₂CH₂CH₂, CH(CH₃)CH₂, CH₂CH(CH₃), CH₂CH₂CH₂CH₂, CH₂CH₂CH₂CH₂CH₂, CH₂CH₂CH₂CH₂CH₂CH₂, and CH₂CH₂CH₂CH₂CH₂CH₂CH₂.

The term “alkylamino” as used herein refers to a straight-chain or branched saturated alkyl group having 1 to 10 carbon atoms, preferably 1 to 4 carbon atoms, more preferably 1 to 3 carbon atoms, which is bonded via a nitrogen atom, e.g. an —NH— group.

The term “dialkylamino” as used herein refers to a straight-chain or branched saturated alkyl group having 1 to 10 carbon atoms, preferably 1 to 4 carbon atoms, more preferably 1 to 3 carbon atoms, which is bonded via a nitrogen atom, which is substituted by another straight-chain or branched saturated alkyl group having 1 to 10 carbon atoms, preferably 1 to 4 carbon atoms, more preferably 1 to 3 carbon atoms, e.g. a methylamino or ethylamino group.

The term “alkylthio “(alkylsulfanyl: alkyl-S—)” as used herein refers to a straight-chain or branched saturated alkyl group having 1 to 10 carbon atoms, preferably 1 to 4 carbon atoms (═C₁-C₄-alkylthio), more preferably 1 to 3 carbon atoms, which is attached via a sulfur atom. Examples include methylthio, ethylthio, propylthio, isopropylthio, and n-butylthio.

The term “haloalkylthio” as used herein refers to an alkylthio group as mentioned above wherein the hydrogen atoms are partially or fully substituted by fluorine, chlorine, bromine and/or iodine. Examples include chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio and pentafluoroethylthio and the like.

The term “alkylsulfinyl” (alkylsulfoxyl: C₁-C₅-alkyl-S(═O)—), as used herein refers to a straight-chain or branched saturated alkyl group (as mentioned above) having 1 to 10 carbon atoms, preferably 1 to 4 carbon atoms (═C₁-C₄-alkylsulfinyl), more preferably 1 to 3 carbon atoms bonded through the sulfur atom of the sulfinyl group at any position in the alkyl group.

The term “alkylsulfonyl” (alkyl-S(═O)₂—) as used herein refers to a straight-chain or branched saturated alkyl group having 1 to 10 carbon atoms, preferably 1 to 4 carbon atoms (═C₁-C₄-alkylsulfonyl), preferably 1 to 3 carbon atoms, which is bonded via the sulfur atom of the sulfonyl group at any position in the alkyl group.

The term “alkylcarbonyl” (C₁-C₆—C(═O)—) refers to a straight-chain or branched alkyl group as defined above, which is bonded via the carbon atom of a carbonyl group (C═O) to the remainder of the molecule.

The term “alkoxycarbonyl” refers to an alkoxygroup group as defined above, which is bonded via the carbon atom of a carbonyl group (C═O) to the remainder of the molecule.

The term “alkylaminocarbonyl” (C₁-C₅—NH—C(═O)—) refers to a straight-chain or branched alkylamino group as defined above, which is bonded via the carbon atom of a carbonyl group (C═O) to the remainder of the molecule. Similarly, the term “dialkylaminocarbonyl” refers to a straight-chain or branched saturated alkyl group as defined above, which is bonded to a nitrogen atom, which is substituted with another straight-chain or branched saturated alkyl group as defined above, which nitrogen atom in turn is bonded via a carbonyl group (C═O) to the remainder of the molecule.

### Preparation Methods

The compounds of formula (I) can be prepared by standard methods of organic chemistry. If certain derivatives cannot be prepared by the processes outlined below, they can be obtained by derivatization of other compounds of formula (I) that are accessible by these methods. The substituted or unsubstituted tricyclic scaffold can for example be prepared by the methods disclosed in WO2013/059559 A2, Examples 1-31 and p. 109-113. The bicyclic moiety of formula (D) on the other hand may be prepared as described in PCT/EP2020/082186. The variables of the following formulae are—unless specified otherwise—as defined for formula (I).

Process 1: For compounds of formula (I) in which A and G are N, such as in compounds of formula (IC), WO2013/059559 A2 describes the condensation reaction of diketones of formula (II) with 1,6-bisamino pyridines of formula (III) to result in 1,8-napthyridines of formula (IV)

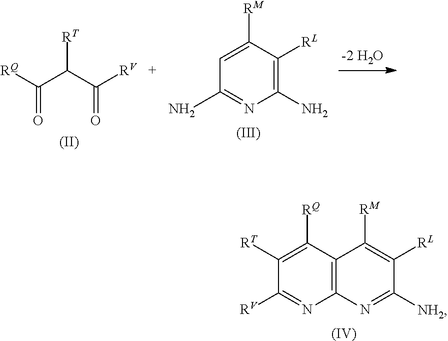

wherein the variables of formulae (II), (III) and (IV) have a meaning as defined for formula (I). Such reactions are usually carried out in the presence of an acid catalyst, e.g. CH₃COOH, at elevated temperatures, e.g. 100-200° C. in an aprotic solvent. Suitable reaction conditions are described in WO2013/059559 A2, paragraphs [00185], or [00189].

Compounds of formula (IV) may then be reacted with 2-bromo-ethanone compounds of formula (V) to result in compounds of formula (VI), which fall under the definition of compounds of formula (I)

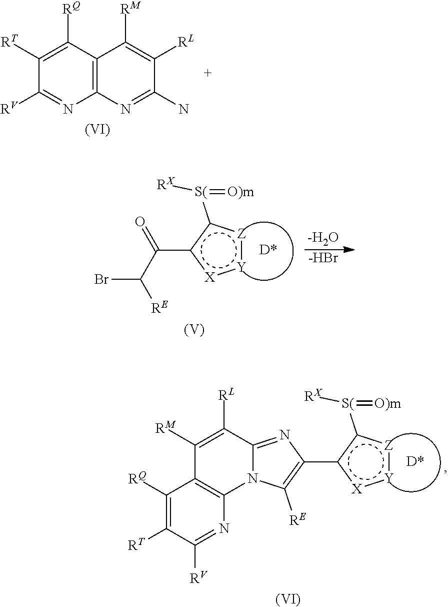

wherein the variables of formulae (IV), (V), and (VI) have a meaning as defined for formula (I). Suitable conditions and solvents for the reaction are described in WO2013/059559 A2, e.g. [00186], or [00190]. Compounds of formula (V) are commercially available or may be prepared as described in WO2016129684 A1, JP 2018177759, PCT/EP2020/082186, WO2018033455 or JP 2018043953.

Process 2: Similarly to the synthesis as described for compounds of formula (VI), compounds of formula (I), wherein A and G are N, J is C, E is CR^{E}, L is CR^{L}, M is CR^{M}, Q is CR^{Q}, T is CR^{T}, V is CR^{V}, and W is CR^{W}, corresponding to compounds of formula (IT),

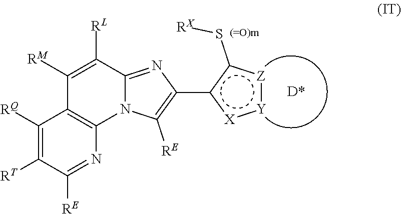

can be prepared from compounds of formula (IVa), which are commercially available,

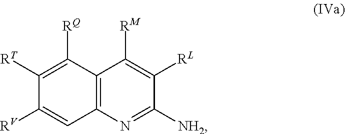

wherein all variables of formulae (IT) and (IVa) are as defined for compounds of formula (I).

Compounds of formula (I), wherein A and G are N, can alternatively be prepared in analogy to WO2013/059559 A2. Typically, a compound of formula VIII is reacted with methyl acrylate in a Heck-type cross-coupling reaction to a compound of formula (IX)

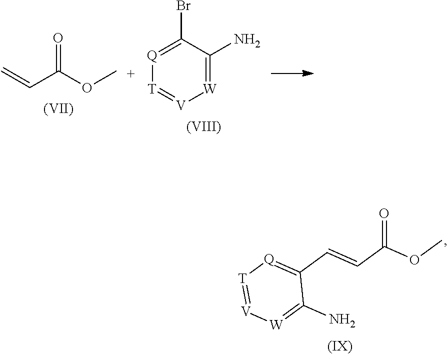

wherein the variables of formulae (VIII) and (IX) have a meaning as defined for formula (I). The reaction is typically carried out in the presence of a Pd(0)-catalyst, which is produced in situ from a Pd(II)-salt in the presence of a suitable ligand, e.g. triphenylphosphane. The reaction may also require the addition of a base, such as an organic base, e.g. triethylamine.
Compounds of formula (IX) may then over a series of reaction steps be converted to compounds of formula (X), as described in WO2013/059559 A2,

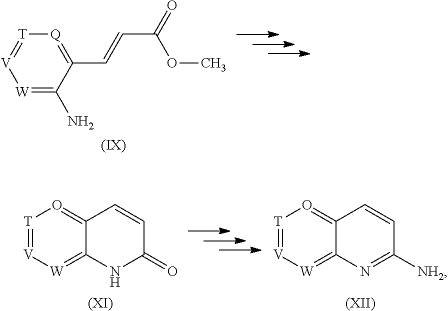

wherein the variables in formulae (IX), (X), and (XII) have a meaning as defined for formula (I).

Compounds of formula (XII) may be reacted with compounds of formula (V) to yield compounds of formula (XIII), falling under the definition of compounds of formula (I)

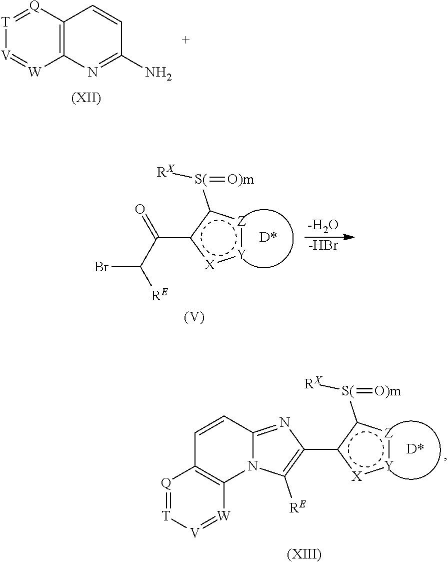

wherein the variables of formulae (V), (XII) and (XIII) have a meaning as defined for formula (I). Reactions of this type have been described in WO2013/059559 A2. The reaction is typically carried out at temperatures of from 50-100° C. in an aprotic polar solvent, e.g. DMF.

Process 3: Compounds of formula (I), wherein A and E are N, and J and G are C, such as in compounds of formulae (IA), (IB), and (ID), may be prepared as follows and as exemplified in the Synthesis Examples. The synthesis typically starts with compounds of formula (XIV)

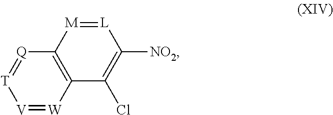

wherein all variables have a meaning as defined for formula (I). Compounds of formula (XIV) are commercially available or may be prepared as described in Bachmann et al, Journal of the American Chemical Society, 1947, vol. 69, p. 365-371. Alternatively, compounds of formula (XIV) may be prepared from compounds of formula (XV) by nitration and chloro-dehydroxylation as described in Gouley et al., Journal of the American Chemical Society, 1947, vol. 69, p. 303-306,

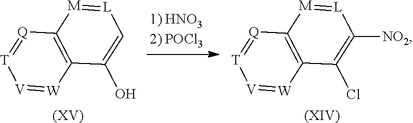

wherein the variables have a meaning as defined for formula (I). Nitration reactions of this type are typically carried out in fuming HNO₃, preferably in the presence of concentrated H₂SO₄ at a temperature of from −5° C. to 30° C.

In a first step, compounds of formula (XV) are then reacted with an amine compound R^{E}—NH₂ to yield compounds of formula (XVI)

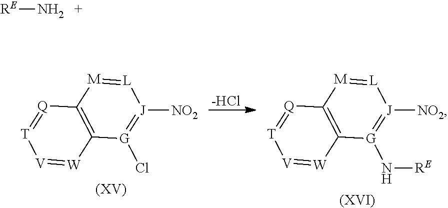

wherein the variables of formulae (XV) and (XVI) are as defined for formula (I). The reaction is typically carried out under elevated temperatures of 40-60° C. in a non-protic solvent, such as an ether, or an aromatic or aliphatic hydrocarbon solvent, e.g. tetrahydrofuran.

In a second step, compounds of formula (XVI) are typically reduced by addition of a reducing agent, such as nascent hydrogen, to form compounds of formula (XVII)

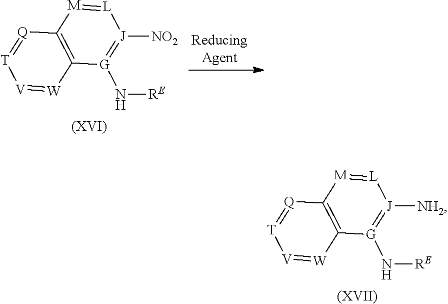

wherein the variables of formulae (XVI) and (XVII) are as defined for formula (I). The nascent hydrogen may for example be produced in situ by the addition of Zn or Fe and CH₃COOH, which also serves as a solvent to the reaction.

In a third step, compounds of formula (XVII) are then reacted with a carbonic acid of formula (XVIII) in the presence of a Coupling Agent to yield compounds of formula (XIX)

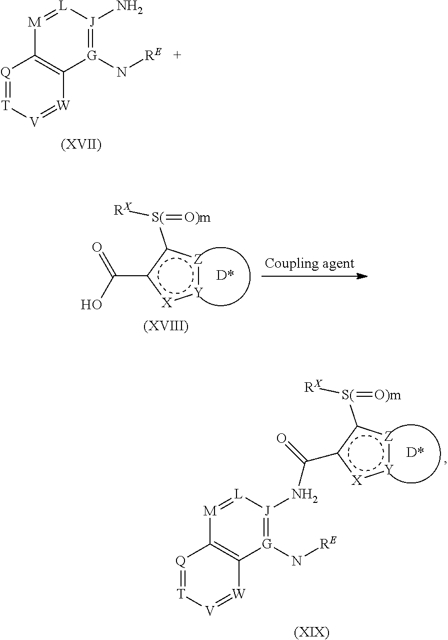

wherein the variables of formulae (XVII), (XVIII) and (XIX) are as defined for formula (I). Typical Coupling Agents are hexafluorophosphate azabenzotriazole tetramethyl uronium (HATU), 3-[Bis(dimethylamino)methyliumyl]-3H-benzotriazol-1-oxide hexafluorophosphate (HBTU), or O-(1H-6-Chlorobenzotriazole-1-yl)-1,1,3,3-tetramethyluronium hexafluorophosphate (HCTU). The reaction may be carried out in a polar aprotic solvent, such as DMF, in the presence of a base. Compounds of formula (XVIII) are commercially available or may be prepared as described in WO2016162318, JP2017033541, JP 2018070585, WO 2018052136, WO2018033455, WO2018050825, WO2015155103, WO2018024657, WO2019043944, or WO2019068572.

In a fourth step, compounds of formula (XIX) are treated with an Acid Catalyst, such as CH₃COOH, or toluene sulfonic acid, to produce compounds of formula (XX), which fall under the definition of compounds of formula (I), in a condensation reaction

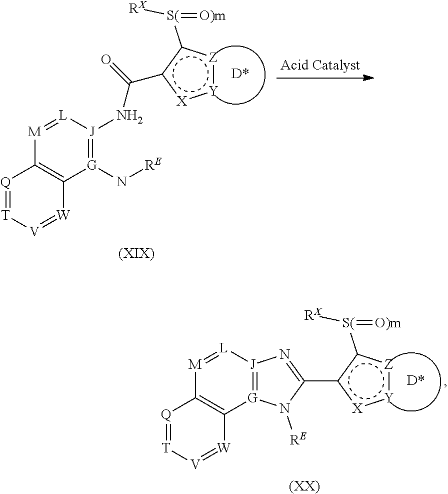

wherein the variables of formulae (XIX), and (XX) have a meaning as defined for formula (I).

Process 4: Compounds of formula (I), wherein A is CH and E is NH may be prepared starting form compounds of formula (XXI)

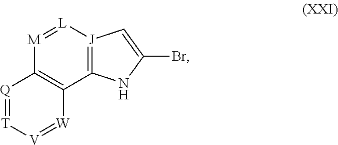

wherein the variables of formula (XXI) have a meaning as defined for formula (I). Compounds of formula XXI are commercially available, or as described in Wang et al., RSC Advances, 2014, vol. 4, issue 51, p. 26918-26923. Compounds of formula (XXI) are also available by methods analogous to those disclosed in WO2013/059559A2, Example 14.

Compounds of formula (XXI) may be reacted with compounds of formula (XXII) in a cross-coupling reaction to yield compounds of formula (XXIII) falling under the definition of compounds of formula (I)

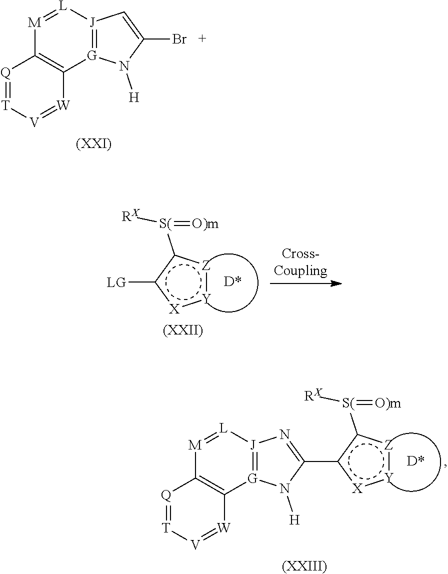

wherein LG is a Leaving Group the other variables of formulae (XXI) and (XXIII) have a meaning as defined for formula (I). Compounds of formula (XXII) are commercially available or may be prepared as described in JP2018024672, JP 2019124548. Typical cross-coupling reactions are Suzuki, Stille and Negishi-type cross-couplings. These reaction are typically carried out in the presence of a Pd(0)-catalyst, which is produced in situ from a Pd(II)-salt in the presence of a suitable ligand, e.g. triphenylphosphane. Suitable Leaving Groups depend on the type of cross-coupling reaction. Leaving Groups suitable in Suzuki-type cross-coupling reactions include boronates, as described in Wesela-Bauman et al., Organic & Biomolecular Chemistry, 2015, vol. 13, issue 11, p. 3268-3279. Suitable Leaving Groups in Stille-type cross-coupling reactions include trialkyl-tin moieties, which are accessible as described in Stille, Angewandte Chemie, 1986, vol. 98, p. 504-519. Suitable Leaving Groups in Negishi-type cross-coupling reactions include zink halogenides, which are accessible as described in Krasovskiy et al, Angewandte Chemie, 2006, volume 45, p. 6040-6044.

Compounds of formula (I), wherein A is NH and E is CR^{E} may be prepared starting form compounds of formula (XXIV)

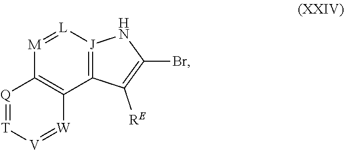

wherein the variables of formula (XXIV) have a meaning as defined for formula (I).

Compounds of formula (XXIV) may be reacted with compounds of formula (XXII) in a cross-coupling reaction as described above to yield compounds of formula (XXV) falling under the definition of compounds of formula (I)

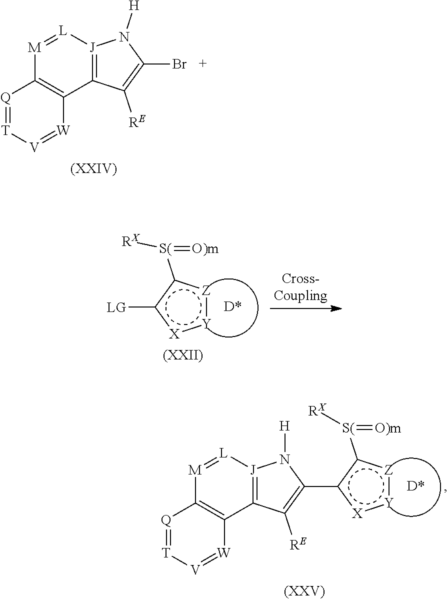

wherein LG is a Leaving Group the other variables of formulae (XXII), (XXIV), (XXV) have a meaning as defined for formula (I). Typical cross-coupling reactions are Suzuki, Stille and Negishi-type cross-couplings. These reaction are typically carried out in the presence of a Pd(0)-catalyst, which is produced in situ from a Pd(II)-salt in the presence of a suitable ligand, e.g. triphenylphosphane. Suitable Leaving Groups depend on the type of cross-coupling reaction. Leaving Groups suitable in Suzuki-type cross-coupling reactions include boronates, as described in Wesela-Bauman et al., Organic & Biomolecular Chemistry, 2015, vol. 13, issue 11, p. 3268-3279. Suitable Leaving Groups in Stille-type cross-coupling reactions include trialkyl-tin moieties, which are accessible as described in Stille, Angewandte Chemie, 1986, vol. 98, p. 504-519. Suitable Leaving Groups in Negishi-type cross-coupling reactions include zink halogenides, which are accessible as described in Krasovskiy et al, Angewandte Chemie, 2006, volume 45, p. 6040-6044.

Process 5: Compounds of formula (I), wherein either A or E is N, may also be available via the Bischler-Möhlau-Indole synthesis. Typical educts are compounds of formula (XXVI) or compounds of formula (XXVII),

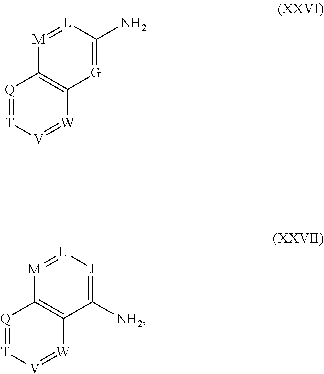

wherein the variables of formulae (XXVI) and (XXVII) have a meaning as defined for formula (I). Compounds of formulae (XXVI) or (XXVII) are commercially available. They are typically reacted with a compound of formula (V) to form compounds of formula (XXVIII) or (XXIX), falling under the definition of compounds of formula (I)

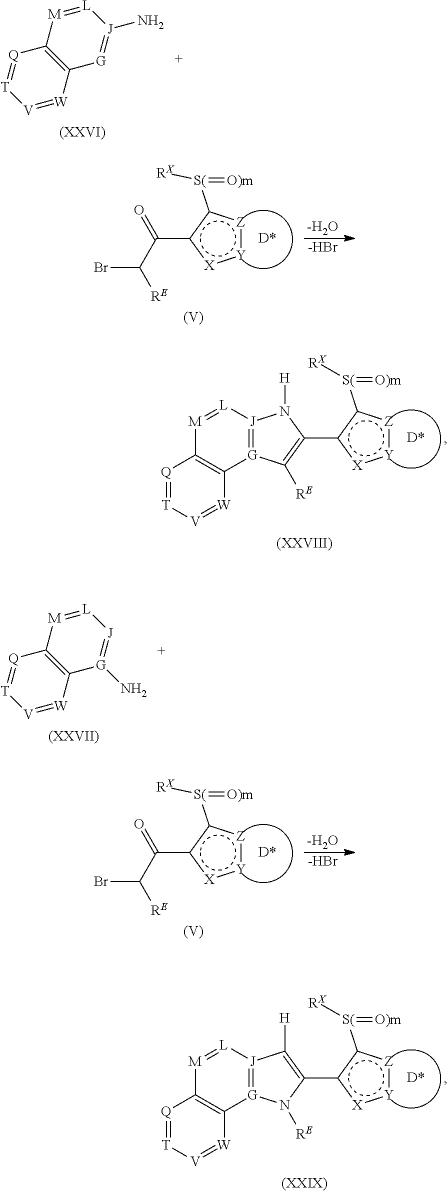

wherein the variables of formulae (XXVI) and (XXVII) have a meaning as defined for formula (I). The reaction is typically carried out in the presence of a base, e.g. Na₂CO₃, under irradiation of microwaves. Reactions of this type have been described by Sridharan et al., Synlett, 2006, p. 91-95. Alternatively, the reaction may be carried out in the presence of a catalyst and a base, such as LiBr and Na₂CO₃, as described by Pchalek et al., Tetrahedron, 2005, vol. 61, issue 3, p. 77-82.

Process 6: Compounds of formula (I), wherein E and J are N, A is CH, and G is C may be prepared from compounds of formula (XXX)

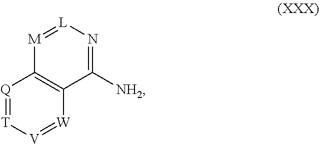

Compounds of formula (XXX) are commercially available or may be prepared as described in WO2003/016275 A1; WO2017/111076 A1; WO2017/014323 A1; WO2014/053208 A1; Van den Haak et al., Journal of Organic Chemistry, 1982, vol. 47, issue 9, p. 1673-7; or US2015/0322090. Compounds of formula (XXX) may be reacted with compounds of formula (V) to yield compounds of formula (XXXI), which fall under the definition of compounds of formula (I)

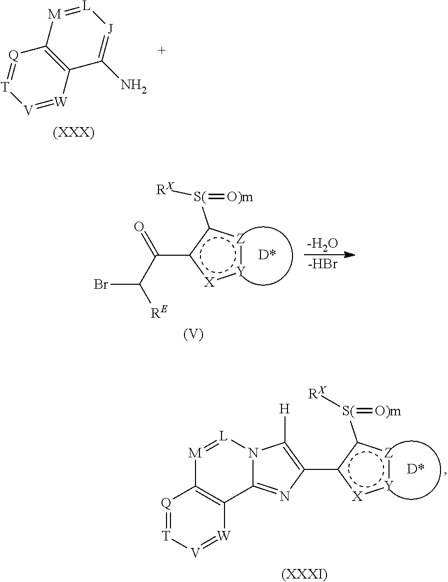

wherein the variables of formulae (V), (XXX) and (XXXI) have a meaning as defined for formula (I). Suitable conditions and solvents for the reaction are described in WO2013/059559 A2, e.g. [00186], or [00190]. Compounds of formula (V) are commercially available or may be prepared as described in Campiani et al, Journal of Medicinal Chemistry, 1998, vol. 41, no. 20, p. 3763-3772.

Process 7: Compounds of formula (I), wherein E is O, may be prepared from compounds of formula (XXXIII) by a Sonogashira-type coupling reaction with methyl prop-2-ynoate to yield compounds of formula (XXXIV)

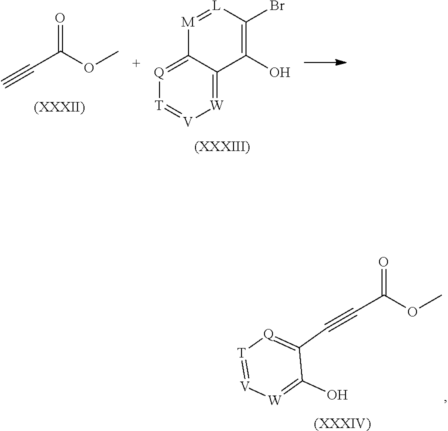

wherein the variables of formulae (XXXIII) and (XXXIV) have a meaning as defined for formula (I). The reaction is typically carried out in an inert solvent the presence of a Cu(I)-salt, such as CuI, a base, such as NaOH, Pd(0), which is produced in situ from Pd(II)Cl₂, and a ligand, such as triphenylphosphine. Compounds of formula (XXXIII) are commercially available.

Compounds of formula (XXXIV) may then be converted to the furan compounds of formula (XXXV) by cycloisomerization

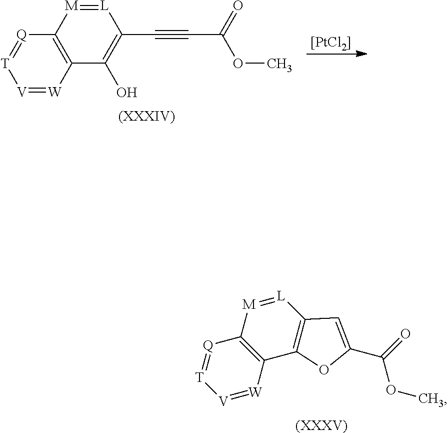

wherein the variables of formulae (XXXIV) and (XXXV) have a meaning as defined for formula (I). The reaction is carried out in the presence of a Pt-catalyst, e.g. PtCl₂ in a non-polar solvent, such as toluene, at elevated temperatures of 50 to 100° C. Reactions of this type have been described by Fürstner et al., Journal of the American Chemical Society, 2005, vol. 127, issue 43, p. 15024-15025.

Compounds of formula (XXXV) may then be reacted with NaOH to generate the carboxylic acid compounds of formula (XXXVI)

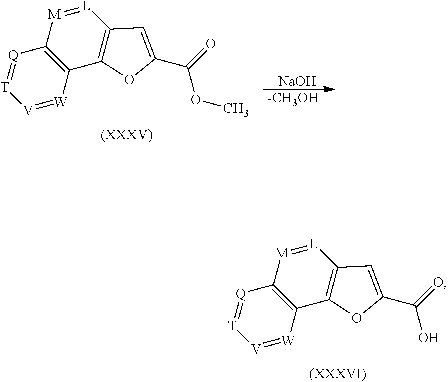

wherein the variables of formulae (XXXV) and (XXXVI) have a meaning as defined for formula (I). The reaction is typically carried out in an aqueous solution of NaOH at a temperature of 50 to 100° C.

Compounds of formula (XXXVI) may be used in a halo-decarboxylation reaction with N(ⁿBu)₄Br₃ to form compounds of formula (XXXVII)

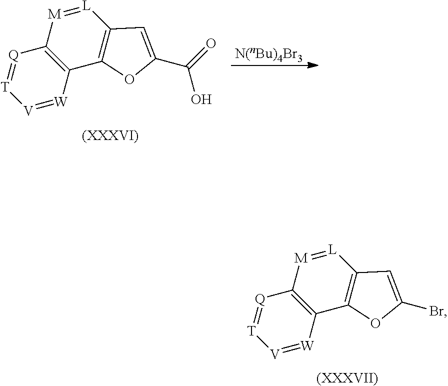

wherein the variables of formulae (XXXVI) and (XXXVII) have a meaning as defined for formula (I). The reaction is typically carried out in a non-protic polar solvent, e.g. acetonitrile, under addition of K₃PO₄, as described in Quibell et al., Chemical Science, 2018, vol. 9, p. 3860.

Compounds of formula (XXXVII) may then be reacted with compounds of formula (XXII) in a Suzuki-type coupling reaction to form compounds of formula (XXXVIII), which fall under the definition of compounds of formula (I)

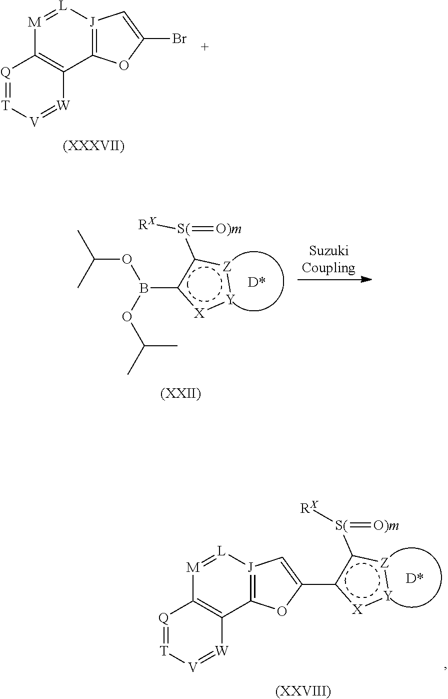

wherein the variables of formulae (XXII), (XXXVII) and (XXXVIII) have a meaning as defined for formula (I). The reaction is typically carried out in the presence of a Pd(0)-catalyst, which is produced in situ from a Pd(II)-salt in the presence of a suitable ligand, e.g. triphenylphosphane. Usually, a base is added to the reaction mixture, such as NaOH.

Process 8: Compounds of formula (I), wherein E is O and A is N, can be prepared from compounds of formula (XXXIX)

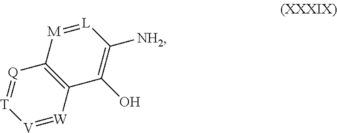

wherein the variables of formula (XXXIX) have a meaning as defined for formula (I). Compounds of formula (XXXIX) are commercially available or may be prepared as described in WO2008/082715 A2, or U.S. Pat. No. 7,364,881 E1.

In a first step, compounds of formula (XXXIX) are reacted with a carbonic acid of formula (XVIII) in the presence of a Coupling Agent to yield compounds of formula (XL), which fall under the definition of compounds of formula (I)

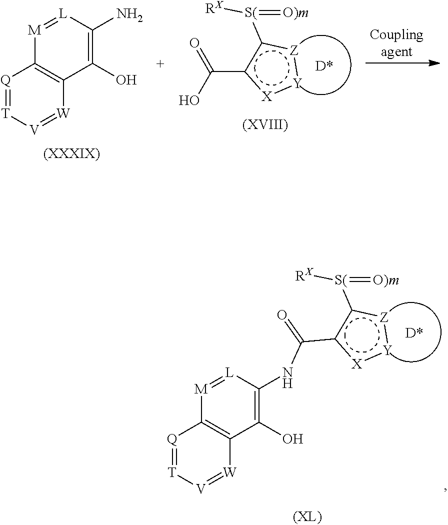

wherein the variables of formulae (XVIII), (XXXIX), and (XL) are as defined for formula (I). Typical Coupling Agents are hexafluorophosphate azabenzotriazole tetramethyl uronium (HATU), 3-[Bis(dimethylamino)methyliumyl]-3H-benzotriazol-1-oxide hexafluorophosphate (HBTU), or O-(1H-6-Chlorobenzotriazole-1-yl)-1,1,3,3-tetramethyluronium hexafluorophosphate (HCTU). The reaction may be carried out in a polar aprotic solvent, such as DMF.

In a second step, compounds of formula (XL) are then cyclized to the oxazol compound of formula (XLI), which fall under the definition of compounds of formula (I), under the addition of POCl₃

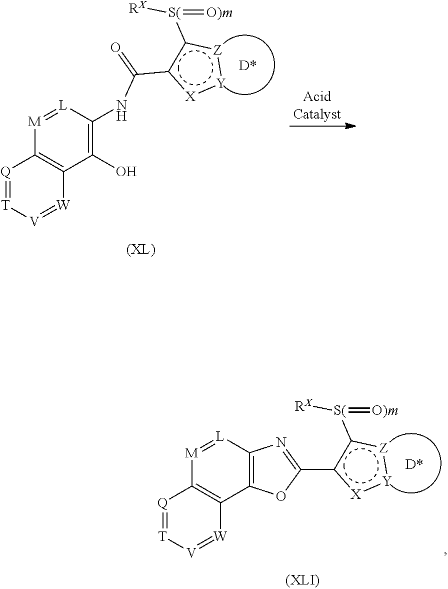

wherein the variables have a meaning as defined for formula (I).

The reaction usually takes place at conditions as described by Li et al., Journal of Organic Chemistry, 2009, vol. 74, issue 9, pp. 3286-3292.

Process 9: Compounds of formula (I), wherein E is S, can be prepared analogously to the compounds of formula (I), wherein E is O. Compounds of formula (I), wherein E is S and A is N, can be prepared starting from compounds of formula (XV). In a first step, compounds of formula (XV) are reacted with Na₂S to yield compounds of formula (XLII)

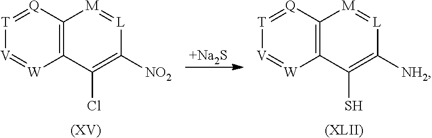

wherein the variables in formulae (XV) and (XLII) have a meaning as defined for formula (I). Reactions of this type have been described by Bachmann et al., Journal of the American Chemical Society, 1947, vol. 69, p. 365-371.

In a second step, compounds of formula (XLII) are then reacted with compounds of formula (XLIII) to yield compounds of formula (XLIV) falling under the definition of compounds of formula (I)

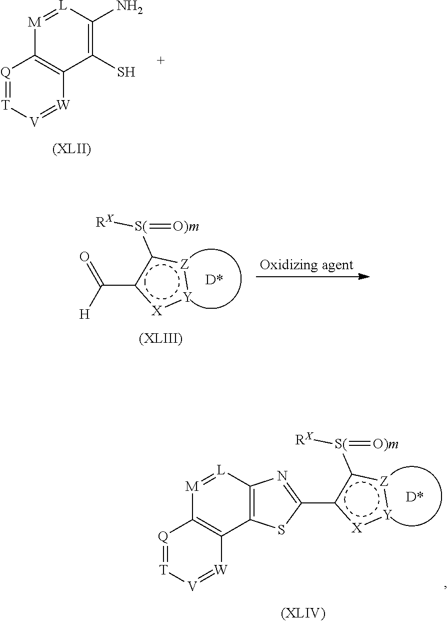

wherein the variables in formulae (XLII), (XLIII) and (XLIV) have a meaning as defined for formula (I). The reaction takes place in the presence of an Oxidizing Agent, e.g. O₂. Reactions of this type have been described in U.S. Pat. No. 4,904,669. Compounds of formula (XLIII) are commercially available or can be prepared from compounds of formula (XVIII).

Process 10: Compounds of formula (I), wherein A, E and G are N, can be prepared starting from compounds of formula (XLV). In a first step, compounds of formula (XLV), which are commercially available, are reacted with ortho-tosylhydroxylamine (TsNH₂) to yield compounds of formula (XLVI)

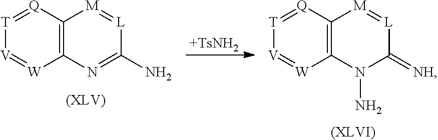

wherein the variables in formulae (XLV) and (XLVI) have a meaning as defined for formula (I). Reactions of this type have been described in Messmer et al., Journal of Organic Chemistry, 1981, vol. 46, p. 843.

Compounds of formula (XLVI) may then be reacted with compounds of formula (XLIII) to yield compounds of formula (XLVII) falling under the definition of compounds of formula (I)

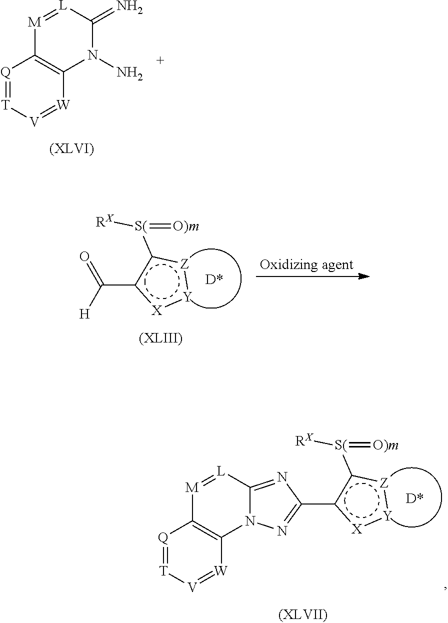

wherein the variables in formulae (XLIII), (XLVI) and (XLVII) have a meaning as defined for formula (I). Reactions of this type have been described in Hoang et al, ARKIVOC, 2001 (ii), 42-50. The reaction is typically carried out in the presence of a base, e.g. KOH, in a protic solvent at a temperature of from 15 to 100° C., preferably at approximately 25° C.

Compounds of formulae (VI), (XIII), (XX), (XXIII), (XXV), (XXVIII), (XXIX), (XXXII), (XXVIII), (XLI), (XLIV), or (XLVII) when m is o or 1 may be oxidized by reaction with an oxidizing agent, e.g. Na₂WO₄, H₂O₂, MnO₂, in a suitable solvent to yield compounds falling under the definition of formula (I). Such oxidation reactions have been described in Voutyritsa et al., Synthesis, vol. 49, issue 4, p. 917-924; Tressler et al, Green Chemistry, vol. 18, issue 18, p. 4875-4878; or Nikkhoo et al., Applied Organometallic Chemistry, 2018, vol. 32, issue 6.

Process 11: Compounds of formula (I), wherein A, E and W are N, and L is CR^{L}, M is CR^{M}, Q is CR^{Q}, T is CR^{T}, and V is CR^{V} can be prepared starting from compounds of formula (XLVIII), which is commercially available,

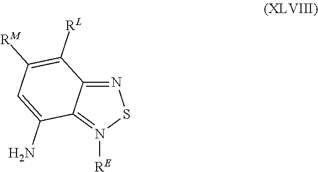

wherein the variables of formula (XLVIII) are as defined for formula (I).
Syntheses of this type have been described in WO2013/059559, p. 143, Example 28. The inventive compounds can be prepared by analogy, wherein the quinoline-7,8-diamine derivative of formula (XLIX) as obtained in step B of Example 28 in WO2013/059558 is further reacted with a compound of formula (XVIII) in the presence of a Coupling Agent, as described above, to yield compounds of formula (L)

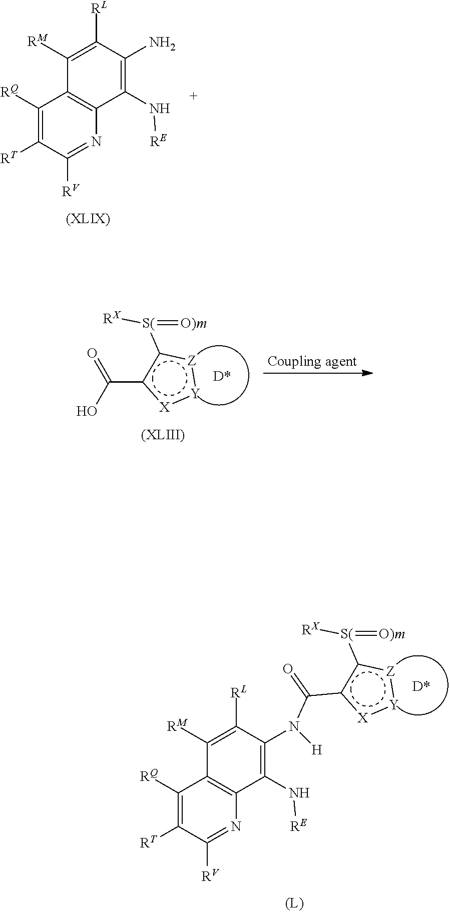

wherein the variables of formulae (XVIII), (XLIX) and (L) are as defined for formula (I).

Just as described for compounds of formula (XIX), compounds of formula (L) may then be treated with an Acid Catalyst to produce compounds of formula (LI), which fall under the definition of compounds of formula (I)

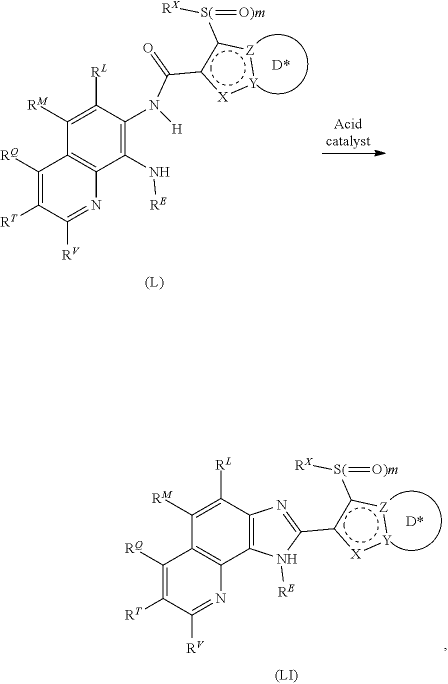

wherein the variables of formulae (L) and (LI) are as defined for formula (I).

Process 12: First step: For compounds of formula (I) in which A and G are N, can be prepared by reacting compound of formula (VI) with (LII) to generate compound (LIII) by using the identical process 1 describe above. Compounds of formula (LII) wherein (LG) can be —Br, —Cl, I, —OTf are commercially available, or may be prepared as described in EP3257853A1, WO2017093180, WO2017125340, WO2018033455, WO2019175045, WO2019175046, Bloorganic & Medicinal Chemistry Letters, 22(5), 1870-1873; 2012,

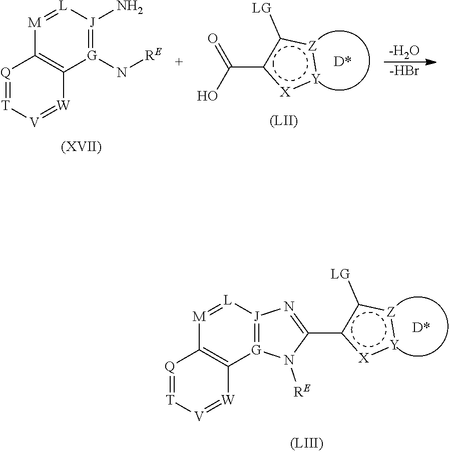

In a second step, compounds of formula (LIII) are then reacted with a compound of formula (LIV) to yield compounds of formula (IV), falling under the definition of compounds of formula (I).

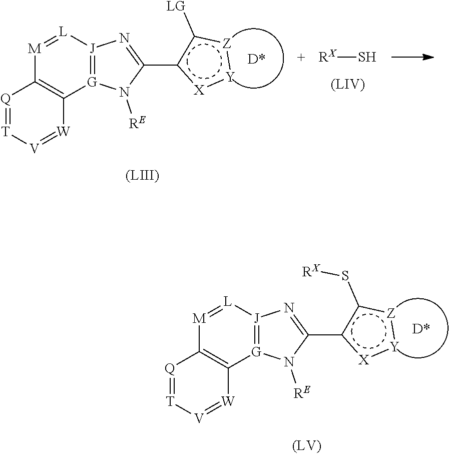

All variables in formulae (LIII), (LIV) and (LV) have a meaning as defined for formula (I). Reactions of this type have been described in WO2016162318A1. The reaction is typically carried out at a temperature of from 15 to 60° C. in an inert solvent in the presence of a base. Suitable solvents are aliphatic hydrocarbons, such as pentane, hexane, cyclohexane, or petrol ether; or aromatic hydrocarbons, such as benzene, toluene, o-, m-, and p-xylene. Mixtures of the above solvents are also possible. Suitable bases are, in general, inorganic bases, preferably alkali metal and alkaline earth metal hydrides, such as LiH, NaH, KH and CaH₂; organic bases, preferably secondary amines, such as pyrrolidine; or tertiary amines, such as diisopropylethylamine, trimethylamine, triethylamine, triisopropylamine and N-methylpiperidine, imidazol, pyridine; substituted pyridines, such as collidine, lutidine and 4-dimethylaminopyridine, and polycyclic amides and amidines, such as 1,8-diazabicycloundec-7-ene (DBU), 1,4-Diazabicyclo[2.2.2]octane (DABCO); or alkali metal salts of secondary amines, such as alkali diisopropylamide, alkali bis(trimethylsilyl)amide, alkali tetramethylpiperidene; alcoholates, such as alkali methanolate, alkali ethanolate, alkali isopropanolate, alkali tert-butanolate; alkali metal—alkyl, and alkali metal—aryl salts, such as n-butyl lithium, tert-butyl lithium, phenyl lithium. The base is typically reacted with compounds of formula (LIV) before compounds of formula (LIII) are added to form the thiolate anion. The bases are generally employed in catalytic amounts; however, they can also be used in equimolar amounts, in excess or, if appropriate, as solvent. The compound (LV) was then subjected for the oxidation of “S” to achieve the compound (XX). By using the similar reaction protocol described in process 12 step 1, compounds (XXXIX), (XLII), (XLVI), and (XLIX) can be reacted separately with (LII) to generate (LVI), (LVII), (LVIII), and (LIX) respectively.

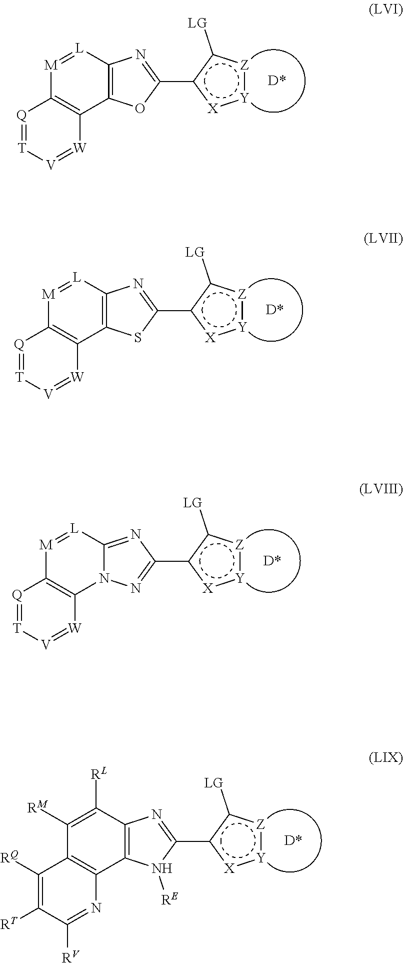

Following the second step described in process 12, compounds (LVI), (LVII), (LVIII), and (LIX) were first reacted with compound with (LIV) to generate (LX), (LXI), (LXII), and (LXIII) respectively. These compounds were further converted to (XLI), (XLIV), (XLVII), and (LI) under oxidative reaction condition as described in process 12.

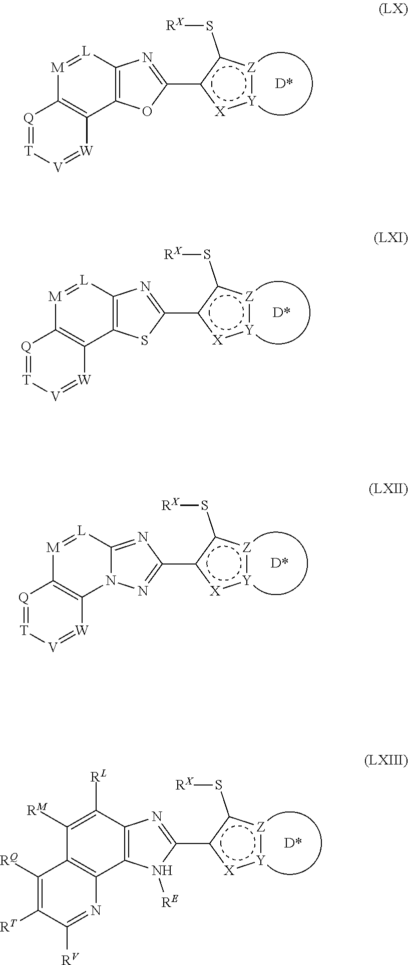

Compounds of formula (I), wherein R⁹ is C(CN)R⁷R⁸ may be prepared in analogy to what has been described for bicyclic compounds in WO2019/068572. Compounds of formula (I), wherein R^{X} is C₃-C₆-cycloalkyl, which is unsubstituted or substituted with one or more, same or different substituents R⁹ may be prepared in analogy to what has been described for bicyclic compounds in WO2019/038195. Compounds of formula (I), wherein D ring partially unsaturated may be prepared in analogy to what has been described in WO2019162174, WO2018033455.

### Preparation Methods

The compounds of formula (I) can be prepared by standard methods of organic chemistry. If certain derivatives cannot be prepared by the processes outlined below, they can be obtained by derivatization of other compounds of formula (I) that are accessible by these methods.

Embodiments and preferred compounds of the present invention for use in pesticidal methods and for insecticidal application purposes are outlined in the following paragraphs. The remarks made below concerning preferred embodiments of the variables of compounds of formula (I) are valid both on their own in combination with each other. The variables of the compounds of formula (I) have the following meanings, these meanings, both on their own and in combination with one another, being particular embodiments of the compounds of the formula (I).

The variable A is CH, N, or NH. In one embodiment, A is N. In another embodiment, A is NH. The variable E is N, NH, O, S, or CR^{E}. In one embodiment, E is NR^{E} or OR^{E}. In another embodiment, A is N or NH, and E is NR^{E} or OR^{E}. In another embodiment, E is NR^{E} or OR^{E} and A is N.

Typically, only one of E or G is N. In one embodiment, both E and G are N. In another embodiment, E is CR^{E} and G is N.

The variables G and J are independently C or N. Typically, both G and J are C. In one embodiment, G is N and J is C, preferably wherein E is N.

The variable L is N or CR^{L}. In one embodiment, the variable L is N. In another embodiment, the variable L is CR^{L}, preferably wherein R^{L} is H, C₁-C₃-alkyl, C₁-C₃-haloalkyl, or C₁-C₃-haloalkoxy, more preferably wherein R^{L} is H, C₁-C₃-fluoroalkyl, or C₁-C₃-fluoroalkoxy, most preferably wherein R^{L} is H, CF₃ or OCF₃, especially preferably wherein R^{L} is H.

The variable M is N or CR^{M}. In one embodiment, the variable M is N. In another embodiment, the variable M is CR^{M}, preferably wherein R^{M} is H, C₁-C₃-alkyl, C₁-C₃-haloalkyl, or C₁-C₃-haloalkoxy, more preferably wherein R^{M} is H, C₁-C₃-fluoroalkyl, or C₁-C₃-fluoroalkoxy, most preferably wherein R^{M} is H, CHF₂, CF₃, OCHF₂, or OCF₃, especially preferably wherein R^{M} is H or CF₃.

The variable Q is N or CR^{Q}. In one embodiment, the variable Q is N. In another embodiment, the variable Q is CR^{Q}, preferably wherein R^{Q} is H, C₁-C₃-alkyl, C₁-C₃-haloalkyl, or C₁-C₃-haloalkoxy, more preferably wherein R^{Q} is H, C₁-C₃-fluoroalkyl, or C₁-C₃-fluoroalkoxy, most preferably wherein R^{Q} is H, CF₃, OCHF₂, or OCF₃, especially preferably wherein R^{Q} is H, CF₃, or OCF₃. In another embodiment, the variable Q is CR^{Q}, preferably wherein R^{Q} is H, C₁-C₃-alkyl, C₁-C₃-alkoxy, C₁-C₃-haloalkyl, or C₁-C₃-haloalkoxy, more preferably wherein R^{Q} is H, C₁-C₃-alkyl, C₁-C₃-fluoroalkyl, C₁-C₃-alkoxy, or C₁-C₃-fluoroalkoxy, most preferably wherein R^{Q} is H, CF₃, OCF₃, OCH₂CH₃, OCHF₂, or OCH₂CF₃.

The variable T is N or CR^{T}. In one embodiment, the variable T is N. In another embodiment, the variable T is CR^{T}, preferably wherein R^{T} is H, C₁-C₃-alkyl, C₁-C₃-haloalkyl, or C₁-C₃-haloalkoxy, more preferably wherein R^{T} is H, C₁-C₃-fluoroalkyl, or C₁-C₃-fluoroalkoxy, most preferably wherein R^{T} is H, or CF₃. In another embodiment, the variable T is CR^{T}, preferably wherein R^{T} is H, C₁-C₃-alkyl, C₁-C₃-haloalkyl, C₁-C₃-alkoxy, or C₁-C₃-haloalkoxy, more preferably wherein R^{T} is H, C₁-C₃-fluoroalkyl, or C₁-C₃-fluoroalkoxy, most preferably wherein R^{T} is H, CF₃, or OCF₃.

The variable V is N or CR^{V}. In one embodiment, the variable V is N. In another embodiment, the variable V is CR^{V}, preferably wherein R^{V} is H, C₁-C₃-alkyl, C₁-C₃-haloalkyl, or C₁-C₃-haloalkoxy, more preferably wherein R^{V} is H, C₁-C₃-fluoroalkyl, or C₁-C₃-fluoroalkoxy, most preferably wherein R^{V} is H, CF₃ or OCF₃, especially preferably wherein R^{V} is H or CF₃, in particular wherein R^{V} is H.

The variable W is N or CR^{W}. In one embodiment, the variable W is N. In another embodiment, the variable W is CR^{W}, preferably wherein R^{W} is H, C₁-C₃-alkyl, C₁-C₃-haloalkyl, or C₁-C₃-haloalkoxy, more preferably wherein R^{W} is H, C₁-C₃-fluoroalkyl, or C₁-C₃-fluoroalkoxy, most preferably wherein R^{W} is H, CF₃ or OCF₃, especially preferably wherein R^{W} is H. In another embodiment, the variable W is CR^{W}, preferably wherein R^{W} is H, C₁-C₃-alkyl, C₁-C₃-haloalkyl, C₁-C₃-haloalkoxy, or C₁-C₃-alkoxy.

Preferred combinations of variables A, E, G, J, L, M, Q, T, V, and W are presented below as formulae (I-A) to (I-JJ), wherein the variables have a meaning as defined for formula (I).

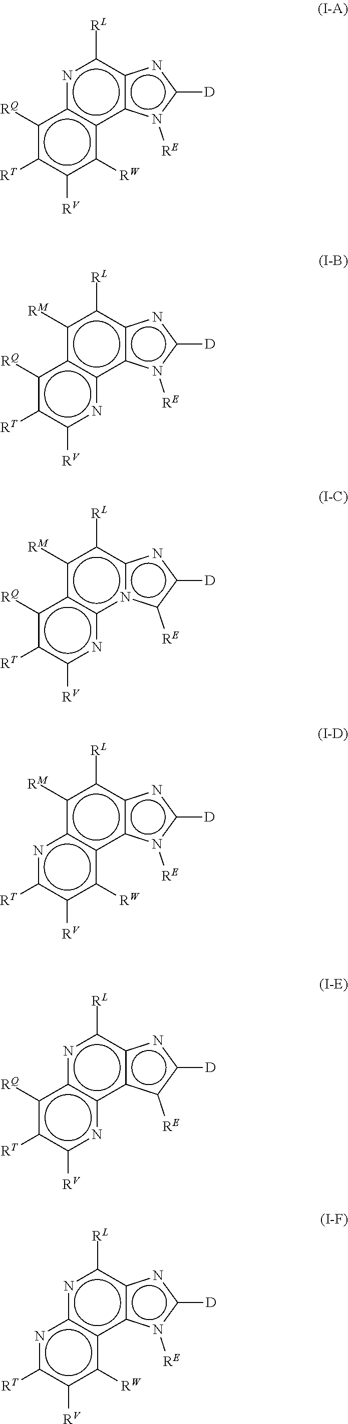

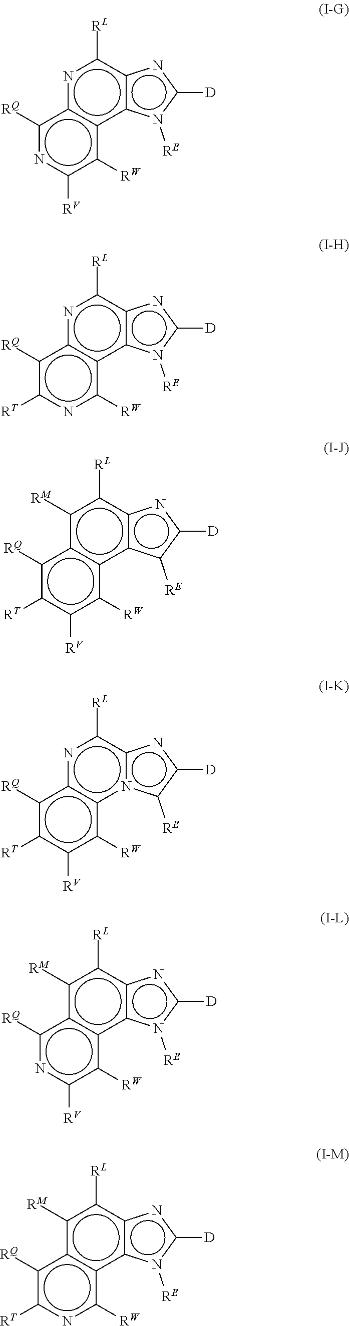

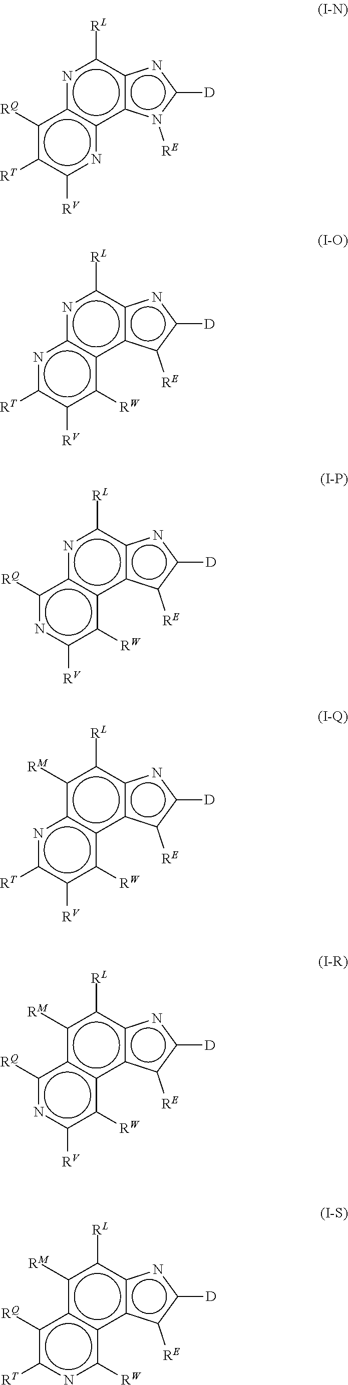

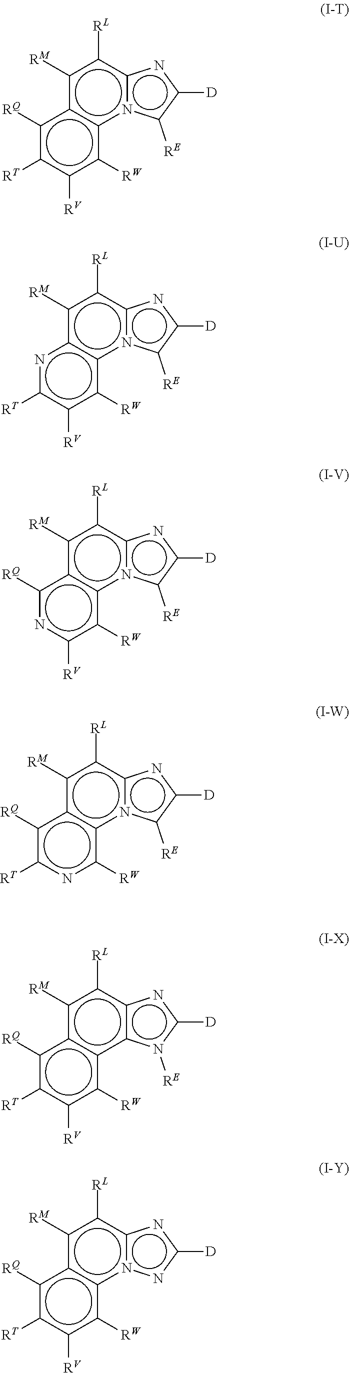

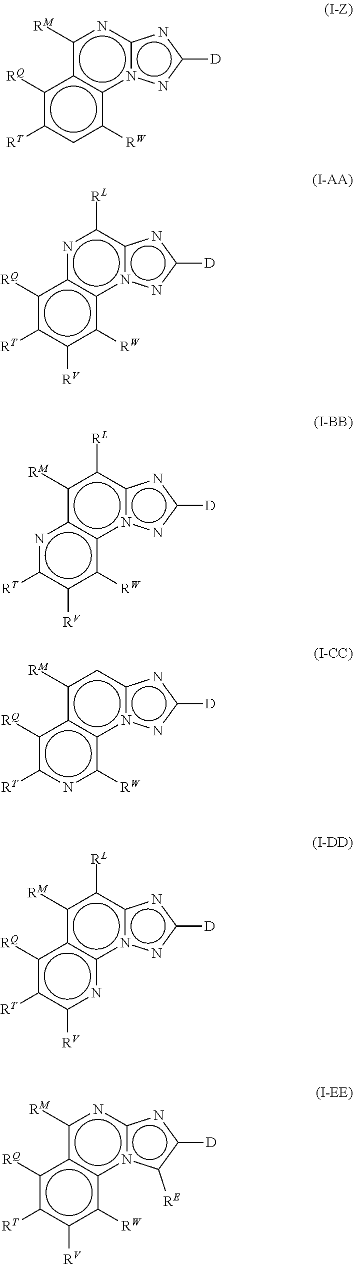

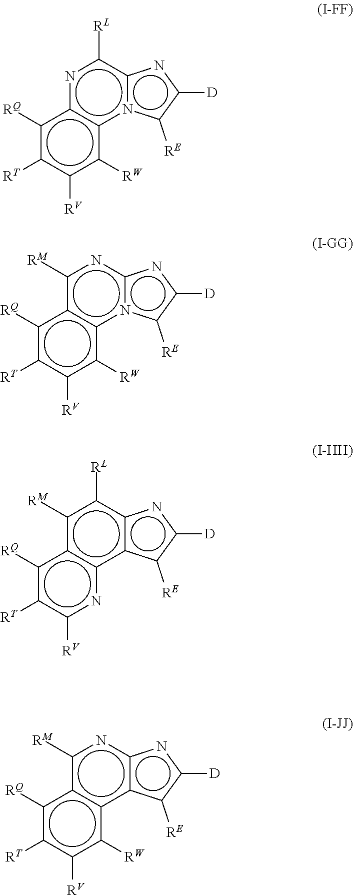

In one embodiment, compounds of formula (I) are compounds of formula (I-A). In another embodiment, compounds of formula (I) are compounds of formula (I-B). In another embodiment, compounds of formula (I) are compounds of formula (I-C). In another embodiment, compounds of formula (I) are compounds of formula (I-D). In another embodiment, compounds of formula (I) are compounds of formula (I-T). In another embodiment, compounds of formula (I) are compounds of formula (I-Y). In another embodiment, compounds of formula (I) are compounds of formulae (I-A), (I-B), (I-C), or (I-D). In another embodiment, compounds of formula (I) are compounds of formulae (I-A), (I-C), or (I-D). In another embodiment, compounds of formula (I) are compounds of formulae (I-A), (I-B), (I-C), or (I-T). In another embodiment, compounds of formula (I) are compounds of formulae (I-A) or (I-C). Typically, at least one of the variables M, Q, T or V is not N.

R^{E}, R^{L}, R^{M}, R^{Q}, R^{T}, R^{V}, and R^{W} independently are selected from H, halogen, N₃, CN, NO₂, SCN, SF₅, C₁-C₆-alkyl, C₁-C₆-alkoxy, C₂-C₆-alkenyl, tri-C₁-C₆-alkylsilyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxyx-C₁-C₄-alkyl, which groups are unsubstituted or substituted with halogen;

C(═O)OR¹, NR²R³, C₁-C₆-alkylen-NR²R³, O—C₁-C₆-alkylen-NR²R³, C₁-C₆-alkylen-CN, NH—C₁-C₆-alkylen-NR²R³, C(═O)NR²R³, C(═O)R⁴, SO₂NR²R³, S(═O)_{q}R⁵, OR⁶, C(═O)R⁶, SR⁶, and benzyl; and phenyl, which is unsubstituted or substituted with one or more, same or different substituents R¹¹.

R^{E} is typically H, halogen, C₁-C₃-alkyl, C₁-C₃-alkoxy, C₂-C₃-alkenyl, C₂-C₃-alkynyl, C₃-C₅-cycloalkyl, which groups are unsubstituted or substituted with halogen. In one embodiment, R^{E} is H, C₁-C₃-alkyl, or C₁-C₃-haloalkyl. In another embodiment, R^{E} is H or CH₃. In another embodiment, R^{E} is CH₃.

R^{L} is typically H, halogen, C₁-C₃-alkyl, C₁-C₃-alkoxy, C₂-C₃-alkenyl, C₂-C₃-alkynyl, C₃-C₅-cycloalkyl, which groups are unsubstituted or substituted with halogen. In one embodiment, R^{L} is H, C₁-C₃-alkyl, C₁-C₃-haloalkyl, C₁-C₃-alkoxy, or C₁-C₃-haloalkoxy. In another embodiment, R^{L} is H or CF₃. In another embodiment, R^{L} is H.

R^{M} is typically H, halogen, C₁-C₃-alkyl, C₁-C₃-alkoxy, C₂-C₃-alkenyl, C₂-C₃-alkynyl, C₃-C₆-cycloalkyl, which groups are unsubstituted or substituted with halogen. In one embodiment, R^{M} is H, C₁-C₃-alkyl, C₁-C₃-haloalkyl, C₁-C₃-alkoxy, or C₁-C₃-haloalkoxy. In another embodiment, R^{M} is H or CF₃.

R^{Q} is typically H, halogen, C₁-C₃-alkyl, C₁-C₃-alkoxy, C₂-C₃-alkenyl, C₂-C₃-alkynyl, C₃-C₅-cycloalkyl, which groups are unsubstituted or substituted with halogen. In one embodiment, R^{Q} is H, C₁-C₃-alkyl, C₁-C₃-haloalkyl, C₁-C₃-alkoxy, or C₁-C₃-haloalkoxy, preferably H, C₁-C₃-haloalkyl, or C₁-C₃-haloalkoxy. In another embodiment, R^{Q} is H, CHF₂, CF₃, OCHF₂, or OCF₃. In another embodiment, R^{Q} is H, CF₃ or OCF₃. In another embodiment, R^{Q} is H, C₁-C₃-alkyl, C₁-C₃-alkoxy, C₁-C₃-haloalkyl, or C₁-C₃-haloalkoxy, more preferably R^{Q} is H, C₁-C₃-alkyl, C₁-C₃-fluoroalkyl, C₁-C₃-alkoxy, or C₁-C₃-fluoroalkoxy, most preferably R^{Q} is H, CF₃, OCF₃, OCH₂CH₃, OCHF₂, or OCH₂CF₃.

R^{T} is typically H, halogen, C₁-C₃-alkyl, C₁-C₃-alkoxy, C₂-C₃-alkenyl, C₂-C₃-alkynyl, C₃-C₆-cycloalkyl, which groups are unsubstituted or substituted with halogen. In one embodiment, R^{T} is H, C₁-C₃-alkyl, C₁-C₃-haloalkyl, C₁-C₃-alkoxy, or C₁-C₃-haloalkoxy, preferably H, C₁-C₃-haloalkyl, or C₁-C₃-haloalkoxy. In another embodiment, R^{T} is H, CHF₂, CF₃, OCHF₂, or OCF₃. In another embodiment, R^{Q} is R^{T} is H, C₁-C₃-haloalkyl, or C₁-C₃-haloalkoxy. In another embodiment, R^{T} is H, or CF₃. In another embodiment, R^{T} is H, C₁-C₃-alkyl, C₁-C₃-haloalkyl, C₁-C₃-alkoxy, or C₁-C₃-haloalkoxy, more preferably R^{T} is H, C₁-C₃-fluoroalkyl, or C₁-C₃-fluoroalkoxy, most preferably R^{T} is H, CF₃, or OCF₃.

R^{V} is typically H, halogen, C₁-C₃-alkyl, C₁-C₃-alkoxy, C₂-C₃-alkenyl, C₂-C₃-alkynyl, C₃-C₅-cycloalkyl, which groups are unsubstituted or substituted with halogen. In one embodiment, R^{V} is H, C₁-C₃-alkyl, C₁-C₃-haloalkyl, C₁-C₃-alkoxy, or C₁-C₃-haloalkoxy, preferably H, C₁-C₃-haloalkyl, or C₁-C₃-haloalkoxy. In another embodiment, R^{V} is H, CHF₂, CF₃, OCHF₂, or OCF₃. In another embodiment, R^{V} is H, CF₃ or OCF₃. In another embodiment, R^{V} is H or CF₃. In another embodiment, R^{V} is H.

R^{W} is typically H, halogen, C₁-C₃-alkyl, C₁-C₃-alkoxy, C₂-C₃-alkenyl, C₂-C₃-alkynyl, C₃-C₅-cycloalkyl, which groups are unsubstituted or substituted with halogen. In one embodiment, R^{V} is H, C₁-C₃-alkyl, C₁-C₃-haloalkyl, C₁-C₃-alkoxy, or C₁-C₃-haloalkoxy. In another embodiment, R^{W} is H, CHF₂, CF₃, OCHF₂, or OCF₃. In another embodiment, R^{W} is H, CF₃ or OCF₃. In another embodiment, R^{W} is H or CF₃. In another embodiment, R^{W} is H.

In one embodiment, R^{M}, R^{Q}, R^{T}, and R^{V} independently are selected from H, C₁-C₆-alkyl, C₁-C₆-alkoxy, C₂-C₆-alkenyl, C₂-C₆-alkynyl, C₃-C₆-cycloalkyl, C₃-C₆-cycloalkoxy, and C₁-C₆-alkyl-S(═O)_{q}, which groups are unsubstituted or substituted with halogen.

In another embodiment, R^{M}, R^{Q}, R^{T}, and R^{V} independently are selected from H, C₁-C₃-alkyl, C₁-C₃-alkoxy, C₂-C₃-alkenyl, C₂-C₃-alkynyl, C₃-C₆-cycloalkyl, and C₃-C₆-cycloalkoxy, which groups are unsubstituted or substituted with halogen. In another embodiment, R^{M}, R^{Q}, R^{T}, and R^{V} independently are selected from H, C₁-C₃-alkyl, and C₁-C₃-alkoxy, which groups are unsubstituted or substituted with halogen.

In another embodiment, R^{M}, R^{Q}, R^{T}, and R^{V} independently are selected from H, C₁-C₃-haloalkyl, and C₁-C₃-haloalkoxy. In another embodiment, R^{M}, R^{Q}, R^{T}, and R^{V} independently are selected from H, C₁-C₃-fluoroalkyl, and C₁-C₃-fluoroalkoxy, wherein at least one substituent R^{M}, R^{Q}, R^{T}, and R^{V} is not H.

In one embodiment, R^{L}, R^{M}, R^{Q}, R^{T}, R^{V}, and R^{W} independently are selected from H, halogen, C₁-C₆-alkyl, C₁-C₆-alkoxy, C₂-C₆-alkenyl, C₂-C₆-alkynyl, C₃-C₆-cycloalkyl, C₃-C₆-cycloalkoxy, and C₁-C₆-alkyl-S(═O)_{q}, which groups are unsubstituted or substituted with halogen.

In another embodiment, R^{L}, R^{M}, R^{Q}, R^{T}, R^{V}, and R^{W} independently are selected from H, halogen, C₁-C₃-alkyl, C₁-C₃-alkoxy, C₂-C₃-alkenyl, C₂-C₃-alkynyl, C₃-C₆-cycloalkyl, and C₃-C₆-cycloalkoxy, which groups are unsubstituted or substituted with halogen. In another embodiment, R^{L}, R^{M}, R^{Q}, R^{T}, R^{V}, and R^{W} independently are selected from H, halogen, C₁-C₃-alkyl, and C₁-C₃-alkoxy, which groups are unsubstituted or substituted with halogen. In another embodiment, R^{L}, R^{M}, R^{Q}, R^{T}, R^{V}, and R^{W} independently are selected from H, C₁-C₃-haloalkyl, and C₁-C₃-haloalkoxy. In another embodiment, R^{L}, R^{M}, R^{Q}, R^{T}, R^{V}, and R^{W} independently are selected from H, halogen, C₁-C₃-alkyl, and C₁-C₃-alkoxy, which groups are unsubstituted or substituted with halogen, wherein at least one variable selected from R^{L}, R^{M}, R^{Q}, R^{T}, R^{V}, and R^{W} is not H. In another embodiment, R^{L}, R^{M}, R^{Q}, R^{T}, R^{V}, and R^{W} independently are selected from H, C₁-C₃-alkyl, and C₁-C₃-alkoxy, which groups are unsubstituted or substituted with halogen. In another embodiment, R^{L} and R^{W} are H, and R^{M}, R^{Q}, R^{T}, and R^{V} are independently H, halogen, C₁-C₃-alkyl, or C₁-C₃-alkoxy, which groups are unsubstituted or substituted with halogen.

In one embodiment, R^{M}, R^{Q}, R^{T}, and R^{V} independently are selected from H, halogen, C₁-C₆-alkyl, C₁-C₆-alkoxy, C₂-C₆-alkenyl, C₂-C₆-alkynyl, C₃-C₆-cycloalkyl, C₃-C₆-cycloalkoxy, and C₁-C₆-alkyl-S(═O)_{q}, which groups are unsubstituted or substituted with halogen.

In another embodiment, R^{M}, R^{Q}, R^{T}, and R^{V} independently are selected from H, halogen C₁-C₃-alkyl, C₁-C₃-alkoxy, C₂-C₃-alkenyl, C₂-C₃-alkynyl, C₃-C₆-cycloalkyl, and C₃-C₆-cycloalkoxy, which groups are unsubstituted or substituted with halogen. In another embodiment, R^{M}, R^{Q}, R^{T}, and R^{V} independently are selected from H, halogen, C₁-C₃-alkyl, or C₁-C₃-alkoxy, which groups are unsubstituted or substituted with halogen. In another embodiment, R^{M}, R^{Q}, R^{T}, and R^{V} independently are selected from H, halogen, C₁-C₃-alkyl, and C₁-C₃-alkoxy, which groups are unsubstituted or substituted with halogen, wherein at least one variable selected from R^{M}, R^{Q}, R^{T}, and R^{V} is not H. In another embodiment, R^{M}, R^{Q}, R^{T}, and R^{V} independently are selected from H, C₁-C₃-alkyl, and C₁-C₃-alkoxy, which groups are unsubstituted or substituted with halogen.

In one embodiment, R^{E} and R^{L} independently are selected from H, halogen, C₁-C₄-alkyl, C₁-C₄-alkoxy, C₂-C₄-alkenyl, and C₂-C₄-alkynyl, which groups are unsubstituted or substituted with halogen. In another embodiment, R^{E} and R^{L} independently are selected from H, C₁-C₃-alkyl, and C₁-C₃-haloalkyl. In another embodiment, R^{E} and R^{L} are independently H, or C₁-C₃-alkyl. In another embodiment, R^{L} is H and R^{E} is H or C₁-C₃-alkyl.

The variable (D) is a fused bicyclic ring of the following formula

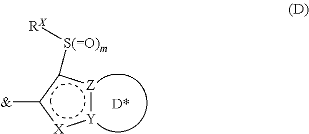

wherein the “&”-symbol signifies the connection to the remainder of formula (I), wherein the dotted circle in the 5-membered ring means that the 5-membered ring may be saturated, partially unsaturated, or fully unsaturated, and wherein the variables have a meaning as defined herein.

The variable X is N, S, O, CR⁷, or NR⁸. In one embodiment, X is N, S, or NR⁸. In another embodiment, X is N. In another embodiment, X is S. In another embodiment, X is NR⁸. In another embodiment, X is O. In another embodiment, X is N or NR⁸.

The variables Y, Z are independently C or N, wherein at least one of the variables selected from Y and Z is C. In one embodiment, Y is N and Z is C. In another embodiment, Y is C and Z is N.

The index m is 0, 1, or 2. In one embodiment, m is 0. In one embodiment, m is 1. In one embodiment, m is 2. In another embodiment, the variable m is 0 or 2.

The index q is 0, 1, or 2. In one embodiment, q is 0. In one embodiment, q is 1. In one embodiment, q is 2. In another embodiment, the variable q is 0 or 2.

R^{X} is C₁-C₆-alkyl, C₃-C₆-cycloalkyl, C₃-C₆-cycloalkyl-C₁-C₄-alkyl, which groups are unsubstituted or substituted with halogen; benzyl or phenyl, wherein the phenyl ring is unsubstituted or substituted with R¹¹. Typically, R^{X} is C₁-C₄-alkyl, which is unsubstituted or substituted with halogen, preferably C₁-C₃-alkyl, or C₁-C₃-haloalkyl, more preferably CH₃CH₂.

R⁷ is H, halogen, OH, CN, NC, NO₂, N₃, SCN, NCS, NCO, SF₅, C₁-C₆-alkyl, C₃-C₆-cycloalkyl, C₂-C₆-alkenyl, C₃-C₆-cycloalkenyl, C₂-C₆-alkynyl, which groups are unsubstituted, or substituted with one or more, same or different substituents R^{G1}; a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents R^{H1}, and wherein said N- and S-atoms are independently oxidized, or non-oxidized;

phenyl, which is unsubstituted, or substituted with one or more, same or different substituents R^{J1}; OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═S)SR^{K1}, OS(═O)R^{K1}, OS(═O)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)ₙR^{V1}, SC(═O)SR^{K1}, SC(═O)NR^{L1}R^{M1}, S(═O)_{q}NR^{L1}R^{M1}, C(═O)R^{P1}, C(═S)R^{P1}, C(═O)NR^{L1}R^{M1}, C(═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, Si(R^{S1})₂R^{T1}.

In one embodiment, R⁷ is H, halogen, OH, CN, NC, NO₂, N₃, SF₅, C₁-C₃-alkyl, C₁-C₃-alkoxy, C₃-C₆-cycloalkyl, C₂-C₃-alkenyl, C₃-C₆-cycloalkenyl, C₂-C₃-alkynyl, which groups are unsubstituted or halogenated. In another embodiment, R⁷ is H, halogen, C₁-C₃-alkyl, C₁-C₃-alkoxy, which groups are unsubstituted or halogenated.

R⁸ is H, CN, C₁-C₆-alkyl, C₃-C₆-cycloalkyl, C₂-C₆-alkenyl, C₃-C₆-cycloalkenyl, C₂-C₆-alkynyl, which groups are unsubstituted, or substituted with one or more, same or different substituents R^{G1};

a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents R^{H1}, and wherein said N- and S-atoms are independently oxidized, or non-oxidized;
phenyl, which is unsubstituted, or substituted with one or more, same or different substituents R^{J1};
OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═S)SR^{K1}, OS(═O)R^{K1}, OS(═O)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)ₙR^{V1}, SC(═O)SR^{K1}, SC(═O)NR^{L1}R^{M1}, S(═O)_{q}NR^{L1}R^{M1}, C(═O)R^{P1}, C(═S)R^{P1}, C(═O)NR^{L1}R^{M1}, (═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, Si(R^{S1})₂R^{T1}.

In one embodiment, R⁸ is H, OH, CN, NC, NO₂, N₃, SF₅, C₁-C₃-alkyl, C₁-C₃-alkoxy, C₃-C₆-cycloalkyl, C₂-C₃-alkenyl, C₃-C₅-cycloalkenyl, C₂-C₃-alkynyl, which groups are unsubstituted or halogenated. In another embodiment, R³ is H, halogen, C₁-C₃-alkyl, C₁-C₃-alkoxy, which groups are unsubstituted or halogenated.

Each R⁹ is independently H, halogen, OH, CN, NC, NO₂, N₃, SCN, NCS, NCO, SF₅, C₁-C₆-alkyl, C₃-C₆-cycloalkyl, C₂-C₆-alkenyl, C₃-C₆-cycloalkenyl, C₂-C₆-alkynyl, C₃-C₅-cycloalkyl-C₁-C₃-alkyl, which groups are unsubstituted, or substituted with one or more, same or different substituents R^{G1}; a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents R^{H1}, and wherein said N- and S-atoms are independently oxidized, or non-oxidized; phenyl, which is unsubstituted, or substituted with one or more, same or different substituents R^{J1}; OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═O)SR^{K1}, OS(═)_{q}R^{K1}, OS(═O)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)ₙR^{V1}, SC(═O)SR^{K1}, SC(═O)NR^{L1}R^{M1}, S(═O)_{q}NR^{L1}R^{M1}, C(O)R^{P1}, C(═S)R^{P1}, C(═O)NR^{L1}R^{M1}, C(═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, Si(R^{S1})₂R^{T1}; or two substituents R⁹ form, together with the ring members of ring D* to which they are bound, a 5- or 6-membered saturated, partially unsaturated, or fully unsaturated carbo- or heterocycle, which carbo- or heterocycle is unsubstituted, or substituted with one or more, same or different substituents R^{J1}, and wherein said heterocycle comprises one or more, same or different heteroatoms O, N, or S.

In one embodiment, each R⁹ is independently H, halogen, OH, CN, NO₂, SF₅, C₁-C₃-alkyl, C₃-C₆-cycloalkyl, C₂-C₃-alkenyl, C₃-C₆-cycloalkenyl, C₂-C₃-alkynyl, C₃-C₆-cycloalkyl-C₁-C₂-alkyl, which groups are unsubstituted, or substituted with one or more, same or different substituents R^{G1}; a 5- to 6-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents R^{H1}, and wherein said N- and S-atoms are independently oxidized, or non-oxidized; phenyl, which is unsubstituted, or substituted with one or more, same or different substituents R^{J1}; OR^{K1}, SR^{K1}, OC(═O)R^{K1}, OC(═O)OR^{K1}, OC(═O)NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)ₙR^{V1}, C(═O)R^{P1}, C(═O)NR^{L1}R^{M1}, C(═O)OR^{K1}; or two substituents R⁹ form, together with the ring members of ring D* to which they are bound, a 5- or 6-membered saturated, partially unsaturated, or fully unsaturated carbo- or heterocycle, which carbo- or heterocycle is unsubstituted, or substituted with one or more, same or different substituents R^{J1}, and wherein said heterocycle comprises one or more, same or different heteroatoms O, N, or S.

In another embodiment, each R⁹ is independently H, halogen, OH, CN, C₁-C₃-alkyl, C₃-C₆-cycloalkyl, C₂-C₃-alkenyl, C₃-C₆-cycloalkenyl, C₂-C₃-alkynyl, C₃-C₆-cycloalkyl-C₁-C₂-alkyl, which groups are unsubstituted, or substituted with one or more, same or different substituents R^{G1}; phenyl, which is unsubstituted, or substituted with one or more, same or different substituents CN, halogen, OR^{K1}, SR^{K1}, OC(═O)R^{K1}, OC(═O)OR^{K1}, OC(═O)NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)ₙR^{V1}, C(═O)R^{P1}, C(═O)NR^{L1}R^{M1}, or C(═O)OR^{K1}.

In another embodiment, each R⁹ is independently H, halogen, OH, CN, C₁-C₃-alkyl, C₁-C₃-alkoxy, C₂-C₃-alkenyl, C₂-C₃-alkynyl, or C₃-C₆-cycloalkyl, which groups are unsubstituted, or substituted with CN or halogen.

In another embodiment, each R⁹ is independently H, halogen, OH, CN, C₁-C₃-alkyl, C₁-C₃-alkoxy, C₂-C₃-alkenyl, or C₂-C₃-alkynyl, which groups are unsubstituted, or halogenated; In another embodiment, each R⁹ is independently H, halogen, OH, CN, C₁-C₃-alkyl, C₁-C₃-alkoxy, C₂-C₃-alkenyl, C₂-C₃-alkynyl, or C₃-C₆-cycloalkyl, which groups are unsubstituted, or substituted with CN or halogen. In another embodiment, each R⁹ is independently C₁-C₃-haloalkyl.

In another embodiment, R⁹ is C₁-C₃-alkyl, C₃-C₆-cylcloalkyl, which groups are substituted with CN, e.g. 1-cyano-cyclopropyl and 1-cyanoisopropyl. In another embodiment, R⁹ is halogen, C₁-C₃-alkyl, which is unsubstituted or substituted with CN or halogen, e.g. 1-cyano-cyclopropyl.

In another embodiment, two substituents R⁹ form, together with the ring members of ring D* to which they are bound, a 5- or 6-membered saturated, partially unsaturated, or fully unsaturated carbo- or heterocycle, which carbo- or heterocycle is unsubstituted, or substituted with one or more, same or different substituents R^{J1}, and wherein said heterocycle comprises one or more, same or different heteroatoms O, N, or S.

Each R^{G1} is independently halogen, OH, CN, NC, NO₂, C₁-C₆-alkyl, C₃-C₆-cycloalkyl, C₃-C₆-cycloalkenyl, which groups are unsubstituted, or substituted with one or more, same or different substituents selected from halogen, OH, CN, C₁-C₃-alkoxy, C₁-C₃-haloalkoxy, and C₁-C₃-alkylcarbonyl; a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents selected from halogen, OH, CN, C₁-C₃-alkoxy, C₁-C₃-haloalkoxy, and C₁-C₃-alkyl-carbonyl, and wherein said N- and S-atoms are independently oxidized, or non-oxidized; phenyl, which is unsubstituted, or substituted with one or more, same or different substituents selected from halogen, OH, CN, NO₂, C₁-C₃-alkyl, C₁-C₃-haloalkyl, C₁-C₃-alkoxy, C₁-C₃-haloalkoxy, and C₁-C₃-alkyl-carbonyl; OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═S)SR^{K1}, OS(═)R^{K1}, OS(O)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)ₙR^{V1}, SC(O)SR^{K1}, SC(═O)NR^{L1}R^{M1}, S(═O)_{q}NR^{L1}R^{M1}, C(═O)R^{P1}, C(═S)R^{P1}, C(═O)NR^{L1}R^{M1}, C(═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, Si(R^{S1})₂R^{T1}.

In one embodiment, each R^{G} is independently halogen, OH, CN, C₁-C₃-alkyl, C₃-C₆-cycloalkyl, C₃-C₆-cycloalkenyl, which groups are unsubstituted, or substituted with one or more, same or different substituents selected from halogen, OH, CN, C₁-C₃-alkoxy, C₁-C₃-haloalkoxy, and C₁-C₃-alkyl-carbonyl; a 5- to 6-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents selected from halogen, OH, CN, C₁-C₃-alkoxy, C₁-C₃-haloalkoxy, and C₁-C₃-alkyl-carbonyl, and wherein said N- and S-atoms are independently oxidized, or non-oxidized; phenyl, which is unsubstituted, or substituted with one or more, same or different substituents selected from halogen, OH, CN, NO₂, C₁-C₃-alkyl, C₁-C₃-haloalkyl, C₁-C₃-alkoxy, C₁-C₃-haloalkoxy, and C₁-C₃-alkyl-carbonyl; OR^{K1}, SR^{K1}, OC(═O)R^{K1}, OC(═O)OR^{K1}, OC(═O)NR^{L1}R^{M1}, ONR^{L1}R^{M1}, ON═CR^{N1}R^{O1}, NR^{L1}R^{M1}, NOR^{K1}, ONR^{L1}R^{M1}, NNR^{L1}, N(R^{L1})C(═O)R^{K1}, N(R^{L1})C(═O)OR^{K1}, C(═O)R^{P1}, C(═S)R^{P1}, C(═O)NR^{L1}R^{M1}, C(═O)OR^{K1}. In one embodiment, each R^{G1} is independently halogen, CN, C₁-C₃-alkyl, C₁-C₃-alkoxy, C₁-C₃-haloalkyl, C₁-C₃-haloalkoxy, or phenyl. In another embodiment, each R^{G1} is independently halogen, C₁-C₃-alkyl, C₁-C₃-alkoxy, C₁-C₃-haloalkyl, or C₁-C₃-haloalkoxy.

Each R^{H1} is independently halogen, CN, NC, NO₂, SCN, NCS, NCO, C₁-C₆-alkyl, C₃-C₆-cycloalkyl, C₃-C₆-cycloalkenyl, which groups are unsubstituted, or substituted with one or more, same or different substituents selected from halogen, OH, CN, C₁-C₁₀-alkoxy, C₁-C₃-haloalkoxy, and C₁-C₃-alkyl-carbonyl;

phenyl, which is unsubstituted, or substituted with one or more, same or different substituents selected from halogen, OH, CN, NO₂, C₁-C₃-alkyl, C₁-C₃-haloalkyl, OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═O)SR^{K1}, OS(═O)R^{K1}, OS(═O)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)ₙR^{V1}, SC(═O)SR^{K1}, SC(═O)NR^{L1}R^{M1}, S(═O)_{q}NR^{L1}R^{M1}, C(═O)R^{P1}, C(═S)R^{P1}, C(═O)NR^{L1}R^{M1}, C(═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, Si(R^{S1})₂R^{T1}; or two geminal substituents R^{H1} form together with the atom to which they are bound a group ═O, ═S, or ═NR^{L}. In one embodiment, each R^{H1} is independently halogen, CN, C₁-C₃-alkyl, C₁-C₃-haloalkyl, C₁-C₃-alkoxy, or C₁-C₃-haloalkoxy.

Each R^{J1} is independently halogen, CN, NC, NO₂, SCN, NCS, NCO, C₁-C₆-alkyl, C₃-C₆-cycloalkyl, C₃-C₆-cycloalkenyl, which groups are unsubstituted, or substituted with one or more, same or different substituents selected from halogen, OH, CN, C₁-C₁₀-alkoxy, C₁-C₃-haloalkoxy, and C₁-C₃-alkyl-carbonyl; phenyl, which is unsubstituted, or substituted with one or more, same or different substituents selected from halogen, OH, CN, NO₂, C₁-C₃-alkyl, C₁-C₃-haloalkyl, OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═S)SR^{K1}, OS(═O)_{q}R^{K1}, OS(═O)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)ₙR^{V1}, SC(═O)SR^{K1}, SC(═O)NR^{L1}R^{M1}, S(═O)NR^{L1}R^{M1}, C═O)R^{P1}, C═S)R^{P1}, C(═O)NR^{L1}R^{M1}, (═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, Si(R^{S1})₂R^{T1}. In one embodiment, each R^{J1} is independently halogen, CN, C₁-C₃-alkyl, C₁-C₃-haloalkyl, C₁-C₃-alkoxy, or C₁-C₃-haloalkoxy.

Each R^{K1} is independently H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with one or more, same or different substituents selected from halogen, CN, NR^{M1}R^{N1}; C(═O)NR^{M1}R^{N1}, C(═O)R^{T1}; or phenyl or benzyl, wherein the phenyl ring is unsubstituted or substituted with one or more, same or different substitutents R^{X1}.

In one embodiment, each R^{K1} is independently C₁-C₃-alkyl, C₂-C₃-alkenyl, C₂-C₃-alkynyl, C₃-C₆-cycloalkyl, C₃-C₆-cycloalkyl-C₁-C₄-alkyl, which groups are unsubstituted or substituted with halogen; phenyl or benzyl, which groups are unsubstituted or substituted with one or more, same or different substituents R^{X1}. In another embodiment, each R^{K1} is independently C₁-C₃-alkyl, C₂-C₃-alkenyl, C₂-C₃-alkynyl, C₃-C₆-cycloalkyl, which groups are unsubstituted or substituted with halogen; phenyl or benzyl, which groups are unsubstituted or substituted with one or more, same or different substituents selected from halogen, C₁-C₃-alkyl, C₁-C₃-alkoxy, C₁-C₃-haloalkoxy, and C₁-C₃-haloalkyl.

Each R^{L1} is independently selected from H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen; C₁-C₆-alkylen-CN; phenyl and benzyl, wherein phenyl groups are unsubstituted or substituted with one or more, same or different substituents R^{X1}.

In one embodiment, each R^{L1} is independently H, C₁-C₃-alkyl, C₂-C₃-alkenyl, C₂-C₃-alkynyl, C₃-C₆-cycloalkyl, C₃-C₆-cycloalkyl-C₁-C₄-alkyl, which groups are unsubstituted or substituted with halogen; phenyl or benzyl, wherein the phenyl groups are unsubstituted or substituted with one or more, same or different substituents R^{X1}. In another embodiment, each R^{L1} is independently H, C₁-C₃-alkyl, C₂-C₃-alkenyl, C₂-C₃-alkynyl, C₃-C₆-cycloalkyl, which groups are unsubstituted or substituted with halogen; phenyl or benzyl, which groups are unsubstituted or substituted with one or more, same or different substituents selected from halogen, C₁-C₃-alkyl, C₁-C₃-alkoxy, C₁-C₃-haloalkoxy, and C₁-C₃-haloalkyl.

Each R^{M1}, R^{R1} is independently H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen; C₁-C₆-alkylen-CN; or phenyl or benzyl, wherein the phenyl ring is unsubstituted or substituted with one or more, same or different substituents R^{X1}.

In one embodiment, each R^{M1}, R^{R1} is independently H, C₁-C₃-alkyl, C₂-C₃-alkenyl, C₂-C₃-alkynyl, C₃-C₆-cycloalkyl, C₃-C₆-cycloalkyl-C₁-C₄-alkyl, which groups are unsubstituted or substituted with halogen; phenyl or benzyl, which groups are unsubstituted or substituted with one or more, same or different substituents R^{X1}. In another embodiment, each R^{M1}, R^{R1} is independently H, C₁-C₃-alkyl, C₂-C₃-alkenyl, C₂-C₃-alkynyl, C₃-C₆-cycloalkyl, which groups are unsubstituted or substituted with halogen; phenyl or benzyl, which groups are unsubstituted or substituted with one or more, same or different substituents selected from halogen, C₁-C₃-alkyl, C₁-C₃-alkoxy, C₁-C₃-haloalkoxy, and C₁-C₃-haloalkyl.

Alternatively, each moiety NR^{M1}R^{R1}, or NR^{L1}R^{M1} may also form an N-bound, saturated 5- to 8-membered heterocycle, which in addition to the nitrogen atom may have 1 or 2 further heteroatoms or heteroatom moieties selected from O, S(═O)_{q} and N—R′, wherein R′ is H or C₁-C₆-alkyl and wherein the N-bound heterocycle is unsubstituted or substituted with one or more, same or different substituents selected from halogen, C₁-C₄-alkyl, C₁-C₄-haloalkyl, C₁-C₄-alkoxy and C₁-C₄-haloalkoxy. In one embodiment, each moiety NR^{M1}R^{R1}, or NR^{L1}R^{M1} may also form an N-bound, saturated 5- to 6-membered heterocycle, wherein the N-bound heterocycle is unsubstituted or substituted with one or more, same or different substituents selected from halogen, C₁-C₃-alkyl, C₁-C₃-haloalkyl, C₁-C₃-alkoxy and C₁-C₃-haloalkoxy.

Each R^{N1} is independently H, halogen, CN, NO₂, SCN, C₁-C₁₀-alkyl, C₃-C₃-cycloalkyl, C₂-C₁₀-alkenyl, C₃-C₃-cycloalkenyl, C₂-C₁₀-alkynyl, which groups are unsubstituted, or substituted with one or more, same or different substituents selected from halogen, C₁-C₆-alkyl, C₁-C₆-alkoxy, C₁-C₆-haloalkyl, and C₁-C₆-haloalkoxy; a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents selected from halogen, C₁-C₃-alkyl, C₁-C₃-alkoxy, C₁-C₃-haloalkyl, and C₁-C₃-haloalkoxy, and wherein said N- and S-atoms are independently oxidized, or non-oxidized; phenyl, which is unsubstituted, or substituted with one or more, same or different substituents selected from halogen, C₁-C₃-alkyl, C₁-C₃-alkoxy, C₁-C₃-haloalkyl, and C₁-C₃-haloalkoxy.

In one embodiment, each R^{N1} is independently C₁-C₃-alkyl, C₂-C₃-alkenyl, C₂-C₃-alkynyl, C₃-C₆-cycloalkyl, which groups are unsubstituted or substituted with halogen; or phenyl, which is unsubstituted or substituted with one or more, same or different substituents selected from halogen, C₁-C₃-alkyl, C₁-C₃-alkoxy, C₁-C₃-haloalkyl, and C₁-C₃-haloalkoxy. In another embodiment, each RN is independently C₁-C₃-alkyl, C₂-C₃-alkenyl, C₂-C₃-alkynyl, C₃-C₆-cycloalkyl, which groups are unsubstituted or substituted with halogen; or phenyl, which is unsubstituted or substituted with one or more, same or different substituents selected from halogen, C₁-C₃-alkyl, C₁-C₃-alkoxy, C₁-C₃-haloalkyl, and C₁-C₃-haloalkoxy.

Each R^{O1} is independently H, C₁-C₄-alkyl, C₁-C₆-cycloalkyl, C₁-C₂-alkoxy-C₁-C₂-alkyl, phenyl, or benzyl; In one embodiment, each R^{O1} is independently H, or C₁-C₃-alkyl.

Each R^{P1} is independently H, C₁-C₅-alkyl, C₂-C₅-alkenyl, C₂-C₅-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen; phenyl or benzyl, wherein the phenyl ring is unsubstituted or substituted with one or more, same or different substituents R^{X1}.

In one embodiment, each R^{P1} is independently C₁-C₃-alkyl, C₂-C₃-alkenyl, C₂-C₃-alkynyl, C₃-C₆-cycloalkyl, C₃-C₆-cycloalkyl-C₁-C₄-alkyl, which groups are unsubstituted or substituted with halogen; phenyl or benzyl, which groups are unsubstituted or substituted with one or more, same or different substituents R^{X1}. In another embodiment, each R^{P1} is independently C₁-C₃-alkyl, C₂-C₃-alkenyl, C₂-C₃-alkynyl, C₃-C₅-cycloalkyl, which groups are unsubstituted or substituted with halogen; phenyl or benzyl, which groups are unsubstituted or substituted with one or more, same or different substituents selected from halogen, C₁-C₃-alkyl, C₁-C₃-alkoxy, C₁-C₃-haloalkoxy, and C₁-C₃-haloalkyl.

Each R^{S1}, R^{T1} is independently H, C₁-C₁₀-alkyl, C₁-C₆-haloalkyl, C₁-C₁₀-alkoxy, C₁-C₄-alkoxy-C₁-C₄-alkyl, C₃-C₃-cycloalkyl, C₃-C₃-halocycloalkyl, C₁-C₄-haloalkoxy-C₁-C₄-alkyl, or phenyl. In one embodiment, each ach R^{S1}, R^{T1} is independently H, C₁-C₃-alkyl, or C₁-C₃-haloalkyl.

Each R^{V1} is independently C₁-C₆-alkyl, C₃-C₆-cycloalkyl, C₃-C₆-cycloalkyl-C₁-C₄-alkyl, which are unsubstituted or substituted with halogen; or phenyl or benzyl, wherein the phenyl ring is unsubstituted or substituted with R^{X1}. In one embodiment, each R^{V1} is independently C₁-C₃-alkyl, C₁-C₃-haloalkyl; or phenyl or benzyl, wherein the phenyl ring is unsubstituted or halogenated.

Each R^{X1} is independently halogen, N₃, OH, CN, NO₂, SCN, SF₅, C₁-C₆ alkyl, C₁-C₆ alkoxy, C₂-C₆ alkenyl, C₂-C₆ alkynyl, C₁-C₆ alkoxy-C₁-C₄ alkyl, C₁-C₆ alkoxy-C₁-C₄ alkoxy, C₃-C₆ cycloalkyl, C₃-C₆ cycloalkoxy, C₃-C₆ cycloalkyl-C₁-C₄ alkyl, C₃-C₆ cycloalkoxy-C₁-C₄ alkyl, which groups are unsubstituted or substituted with halogen. In one embodiment, each R^{X1} is independently halogen, OH, CN, NO₂, C₁-C₃-alkyl, C₁-C₃-alkoxy, C₂-C₃ alkenyl, C₂-C₃-alkynyl, C₃-C₆-cycloalkyl, which groups are unsubstituted or substituted with halogen. In another embodiment, each R^{X1} is independently halogen, C₁-C₃-alkyl, C₁-C₃-alkoxy, C₂-C₃ alkenyl, C₂-C₃-alkynyl, which groups are unsubstituted or substituted with halogen. In another embodiment, each R^{X1} is independently halogen, C₁-C₃-alkyl, or C₁-C₃-haloalkyl.

The variable D* represents a 5- or 6-membered saturated, partially unsaturated, or fully unsaturated carbo- or heterocycle, which carbo- or heterocycle includes the atoms Y and Z as ring members and is unsubstituted, or substituted with one or more, same or different substituents R⁹, and wherein said heterocycle comprises 0, 1, 2, or 3, same or different heteroatoms O, N, or S in addition to those that may be present as ring members Y and Z.

In one embodiment, the variable D* represents a 6-membered saturated, partially unsaturated, or fully unsaturated carbo- or heterocycle, which carbo- or heterocycle includes the atoms Y and Z as ring members and is unsubstituted, or substituted with one or more, same or different substituents R⁹, and wherein said heterocycle comprises 0, 1, or 2, same or different heteroatoms O, N, or S in addition to those that may be present as ring members Y and Z.

In another embodiment, the variable D* represents a 6-membered saturated, partially unsaturated, or fully unsaturated carbo- or heterocycle, which carbo- or heterocycle includes the atoms Y and Z as ring members and is unsubstituted, or substituted with one or more, same or different substituents R⁹, and wherein said heterocycle comprises none or one N-atoms in addition to those that may be present as ring members Y and Z.

In another embodiment, the variable D* represents a 6-membered partially or fully unsaturated carbocycle, which carbo- or heterocycle includes the atoms Y and Z as ring members and is unsubstituted, or substituted with one or more, same or different substituents R⁹. In another embodiment, the variable D* represents a 6-membered partially or fully unsaturated heterocycle, which heterocycle includes the atoms Y and Z as ring members and is unsubstituted, or substituted with one or more, same or different substituents R⁹, and wherein said heterocycle comprises C, same or different heteroatoms O, N, or S in addition to those that may be present as ring members Y and Z.

In one embodiment, the variable D* represents a 5-membered saturated, partially unsaturated, or fully unsaturated carbo- or heterocycle, which carbo- or heterocycle includes the atoms Y and Z as ring members and is unsubstituted, or substituted with one or more, same or different substituents R⁹, and wherein said heterocycle comprises one or more, same or different heteroatoms O, N, or S in addition to those that may be present as ring members Y and Z. In another embodiment, the variable D* represents a 5-membered partially or fully unsaturated carbocycle, which carbo- or heterocycle includes the atoms Y and Z as ring members and is unsubstituted, or substituted with one or more, same or different substituents R⁹. In another embodiment, the variable D* represents a 5-membered partially or fully unsaturated heterocycle, which heterocycle includes the atoms Y and Z as ring members and is unsubstituted, or substituted with one or more, same or different substituents R⁹, and wherein said heterocycle comprises one or more, same or different heteroatoms O, N, or S in addition to those that may be present as ring members Y and Z.

The variable X is N, S, O, CR⁷, or NR⁸. In one embodiment, the variable X is N. In another embodiment, the variable X is NR⁸. In another embodiment, the variable X is O. In another embodiment, the variable X is S.

The variables Y, Z are independently C or N, wherein at least one of the variables selected from Y and Z is C. In one embodiment, Y is N and Z is C. In another embodiment, Z is N and Y is C.

In another embodiment, X and Y are N, and Z is C.

Accordingly, the fused bicyclic ring D may be presented by a formula D1 to D51

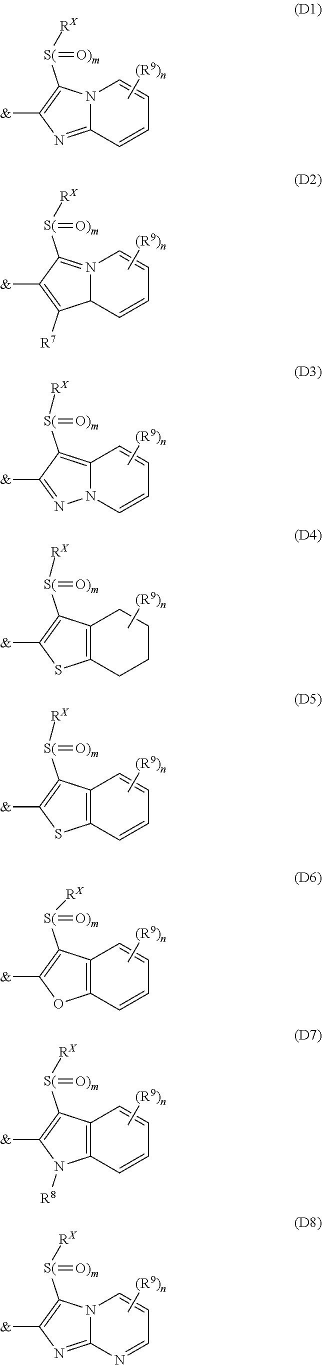

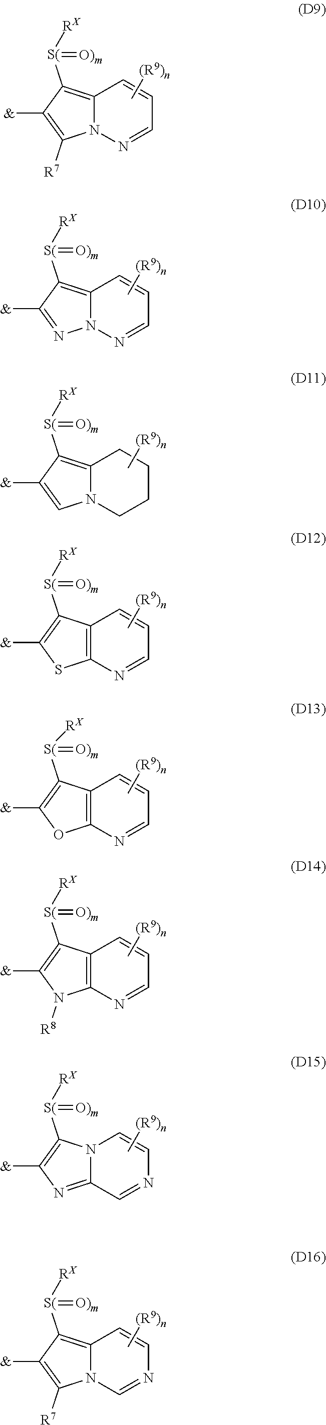

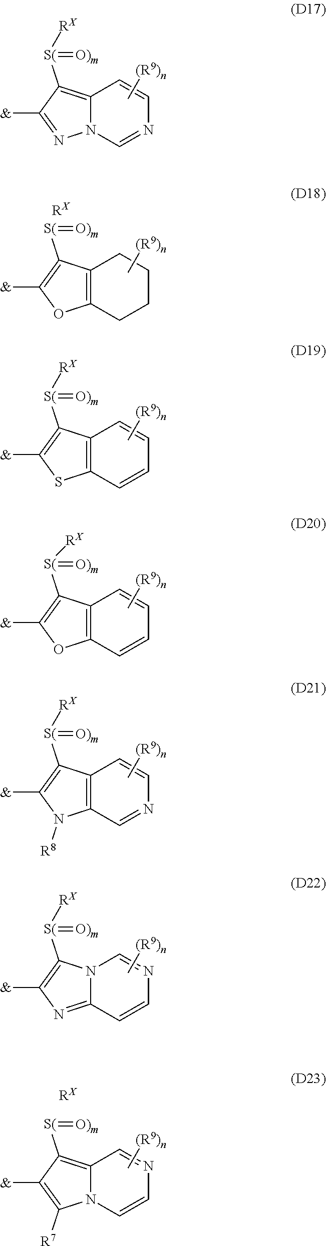

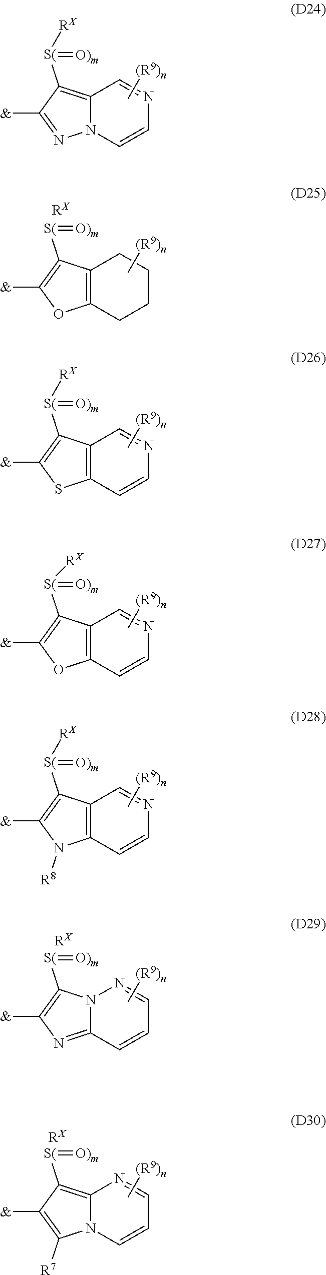

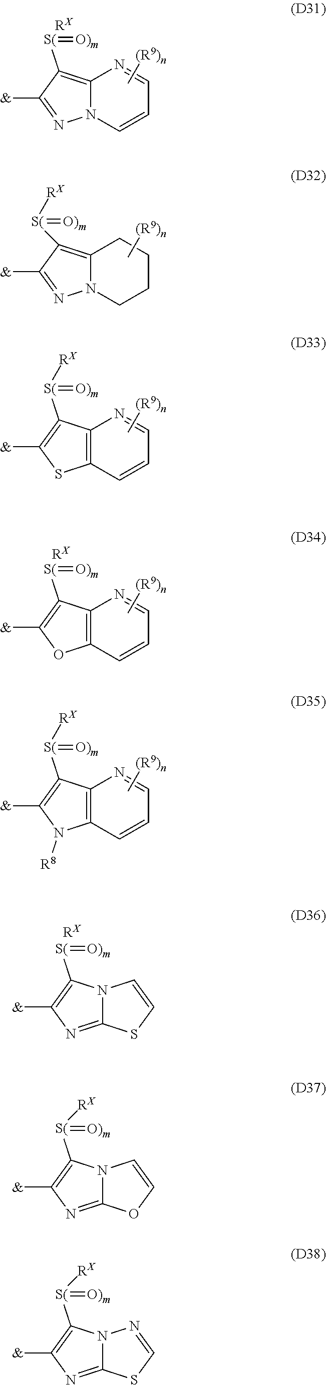

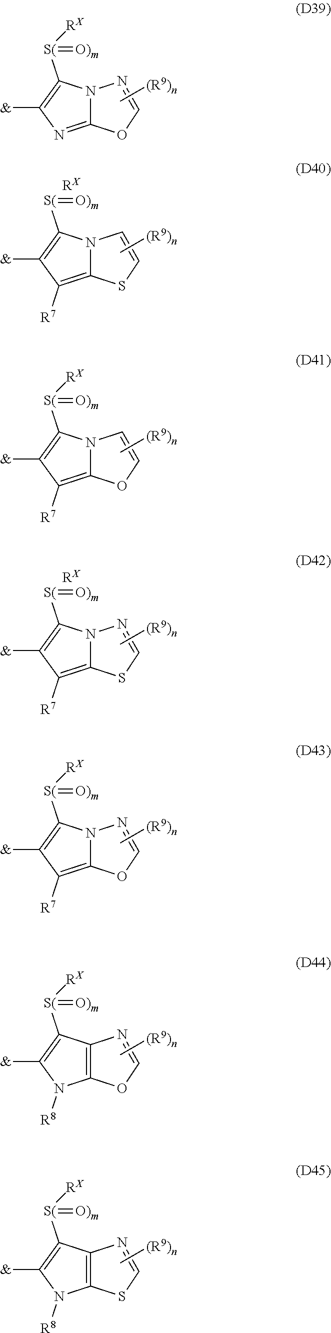

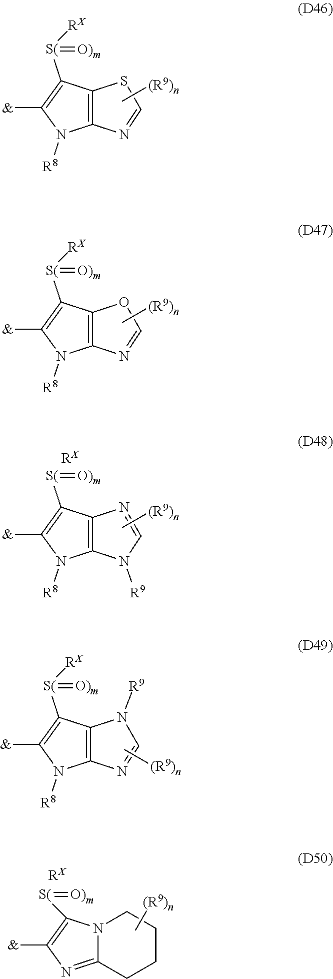

wherein the index n is 0, 1, 2, 3, or 4, preferably 1, and wherein all other variables have a meaning as defined for formula (I). In one embodiment, the bicyclic ring D is of formula (D1), (D3), (D8) and (D50), preferably wherein the index n is 0 or 1. For the avoidance of doubt: substituent(s) R⁹ are bound to a ring member of ring D*. The position of R⁹ may be described by the following scheme: Formulae (D.A) and (D.B) display the alternatives of the ring D* being either a 6-membered or 5-membered ring, respectively

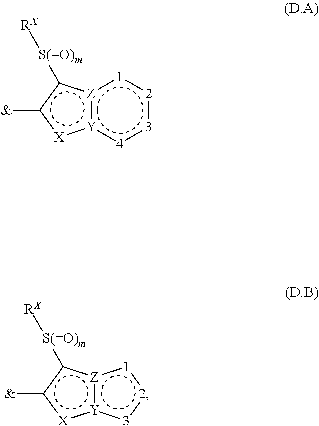

wherein the numbers 1, 2, 3, and 4 each independently denominate the position of a specific ring member, wherein the identity of said ring members is as described herein for formula (I), wherein the “&”-symbol signifies the connection to the remainder of formula (I), wherein the dotted circles in the fused rings means that fused rings may be saturated, partially unsaturated, or fully unsaturated; and wherein the other variables are defined as for formula (I).

Accordingly, the position x of a substituent R⁹ of a ring D1 to D51 will be indicated by the respective suffix “.x”, such as D1.1, D1.2, D1.3, or D1.4.

For example, a fused bicyclic ring D1 having one substituent R⁹ at position 2 would correspond to the ring (D1.2)

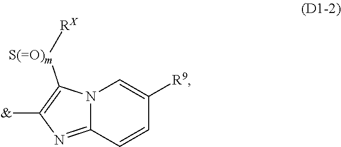

wherein all variables have a meaning as defined for formula (I).

In one embodiment, the compounds of formula (I) are compounds of formula (I-A), (I-B), (I-C), or (I-D) wherein
R^{E}, R^{L}, R^{M}, R^{Q}, R^{T}, R^{V}, R^{W} independently are selected from H, C₁-C₃-alkyl, C₁-C₃-alkoxy, C₂-C₃-alkenyl, and C₂-C₃-alkynyl, which groups are unsubstituted or substituted with halogen;
D is D1, D3, D8 or D50;
R^{X} is C₁-C₃-alkyl, which is unsubstituted or substituted with halogen.
m is 0, or 2;
n is 0, 1, or 2.

In another embodiment, the compounds of formula (I) are compounds of formula (I-A), (I-C), or (I-D) wherein
R^{E}, R^{L}, R^{M}, R^{Q}, R^{T}, R^{V}, R^{W} independently are selected from H, C₁-C₃-alkyl, C₁-C₃-alkoxy, C₂-C₃-alkenyl, and C₂-C₃-alkynyl, which groups are unsubstituted or substituted with halogen;
D is D1, D3, D8 or D50;
R^{X} is C₁-C₃-alkyl, which is unsubstituted or substituted with halogen.
m is 0, or 2;
n is 0, 1, or 2.

In another embodiment, the compounds of formula (I) are compounds of formula (I-A), (I-C), or (I-D) wherein
R^{E}, R^{L}, R^{M}, R^{Q}, R^{T}, R^{V}, R^{W} independently are selected from H, SCF₃, C₁-C₃-alkyl, C₁-C₃-alkoxy, which groups are unsubstituted or substituted with halogen;
D is D1, D3, D8 or D50, preferably D1.2, D3.2, D8.2, D50.2, D1.3, D3.3, D8.3, D50.3, more preferably D1.2, D3.2, D8.2 or D50.2;
R^{X} is C₁-C₃-alkyl, which is unsubstituted or substituted with halogen;
R⁹ is halogen;
   C₁-C₃-alkyl, C₁-C₃-alkoxy, cyclopropyl, which are unsubstituted or substituted with one or more, same or different substituent selected from halogen and CN;
m is 0, or 2;
n is 0, or 1.

In another embodiment, the compounds of formula (I) are compounds of formula (I-A), (I-C), or (I-D) wherein
R^{E}, R^{L}, R^{M}, R^{Q}, R^{T}, R^{V}, R^{W} independently are selected from H, C₁-C₃-alkyl, C₁-C₃-alkoxy, which groups are unsubstituted or substituted with halogen;
D is D1, D3, D8 or D50, preferably D1.2, D3.2, D8.2, D50.2, D1.3, D3.3, D8.3, D50.3, more preferably D1.2, D3.2, D8.2 or D50.2;
R^{X} is C₁-C₃-alkyl, which is unsubstituted or substituted with halogen;
R⁹ is halogen;
   C₁-C₃-alkyl, which is unsubstituted or substituted with one or more, same or different substituent selected from halogen and CN;
m is 0, or 2;
n is 0, or 1.

In another embodiment, the compounds of formula (I) are compounds of formula (I-A), (I-C), or (I-D) wherein
R^{M}, R^{Q}, R^{T}, R^{V}, R^{W} independently are selected from H, SCF₃, C₁-C₃-alkyl, C₁-C₃-alkoxy, which groups are unsubstituted or substituted with halogen;
R^{L} is H;
R^{E} is H, CH₃, which is unsubstituted or halogenated, preferably H or CH₃;
D is D1, D3, D8 or D50, preferably D1.2, D3.2, D8.2, D50.2, D1.3, D3.3, D8.3, D50.3, more preferably D1.2, D3.2, D8.2 or D50.2;
R^{X} is C₁-C₃-alkyl, which is unsubstituted or substituted with halogen;
R⁹ is halogen;
   C₁-C₃-alkyl, C₁-C₃-alkoxy, cyclopropyl, which are unsubstituted or substituted with one or more, same or different substituent selected from halogen and CN;
m is 0, or 2;
n is 0, or 1.

In another embodiment, the compounds of formula (I) are compounds of formula (I-A), (I-C), or (I-D) wherein
R^{E}, R^{M}, R^{Q}, R^{T}, R^{V}, independently are selected from H, C₁-C₃-alkyl, C₁-C₃-alkoxy, which groups are unsubstituted or substituted with halogen;
R^{L}, R^{W} are H;
D is D1, D3, D8 or D50, preferably D1.2, D3.2, D8.2, D50.2, D1.3, D3.3, D8.3, D50.3, more preferably D1.2, D3.2, D8.2 or D50.2;
R^{X} is C₁-C₃-alkyl, which is unsubstituted or substituted with halogen;
R⁹ is C₁-C₃-alkyl, which is unsubstituted or substituted with halogen;
m is 0, or 2;
n is 0, or 1.

Particularly preferred are the compounds of formula IA-D1 to IC1-D50 below, wherein the variables are as defined herein.

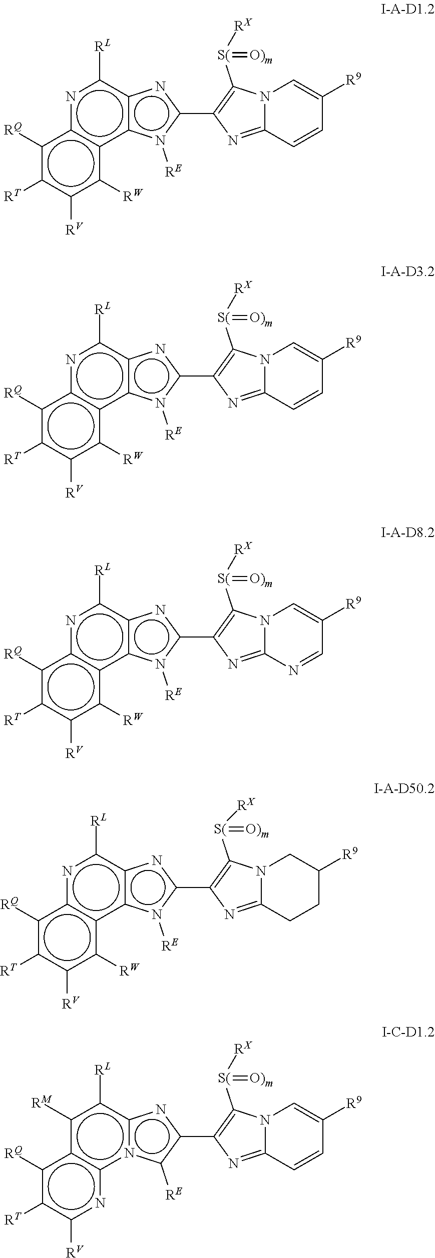

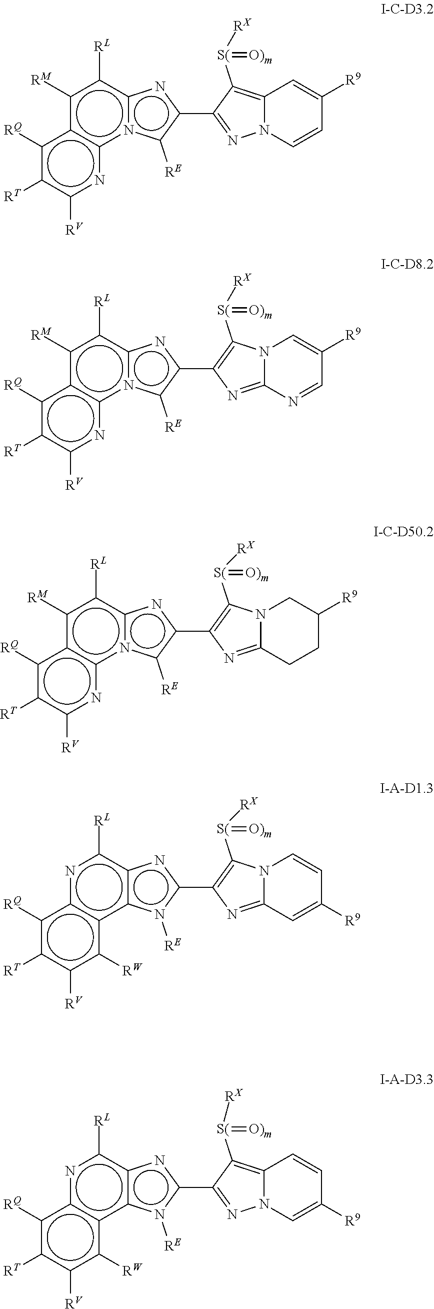

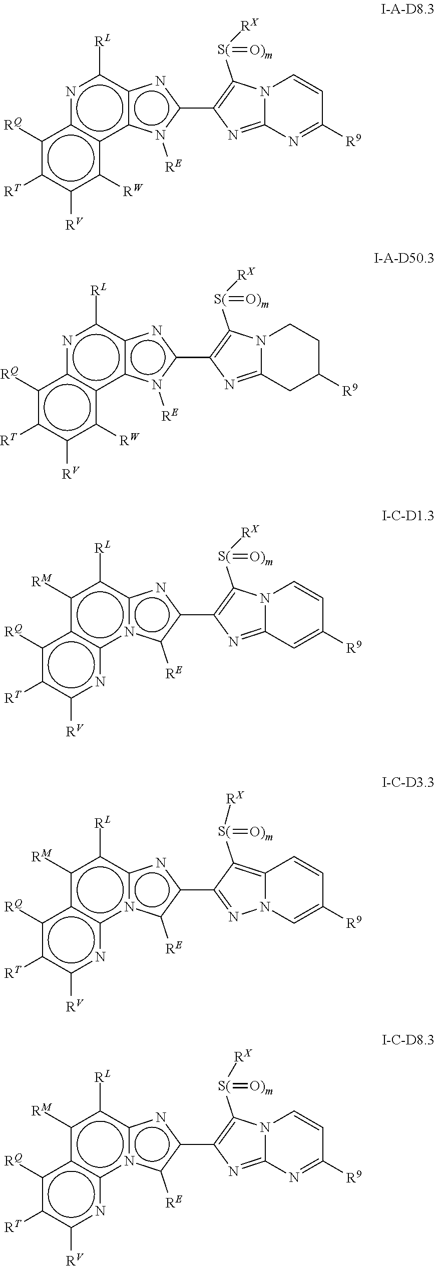

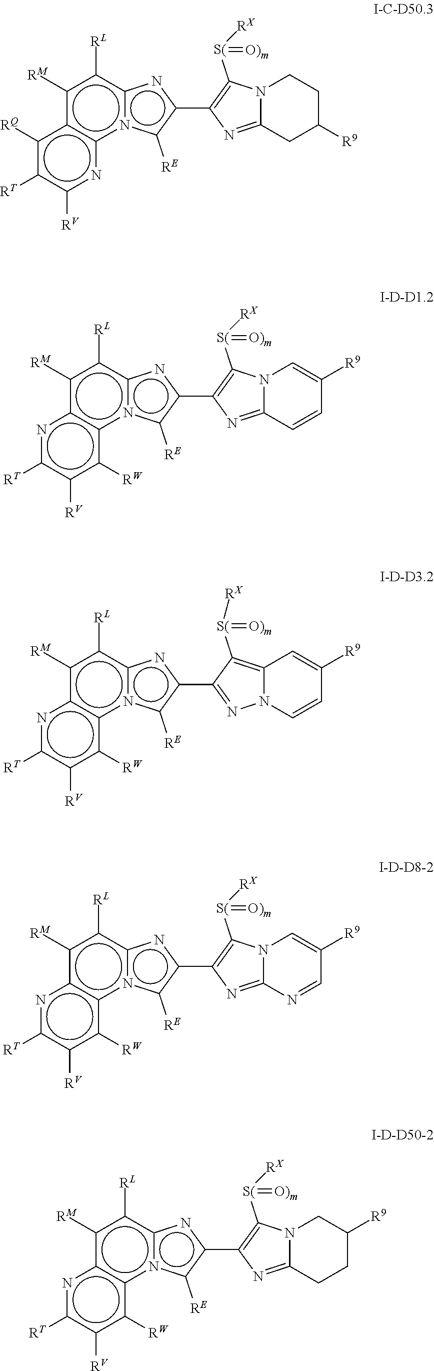

Also particularly preferred are the compounds as disclosed in Table 1 to Table 383 wherein the combinations of other variables R^{Q}, R^{T}, and R⁹—if present—are as defined in each line of Table B

Table 1. Compounds of formula I-A-D1.2, wherein R^{L}, R^{V}, R^{W}, R^{E} are H, R^{X} is CH₃, and m is 2.
Table 2. Compounds of formula I-A-D1.2, wherein R^{L}, R^{V}, R^{W}, R^{E} are H, R^{X} is C₂H₅, and m is 2.
Table 3. Compounds of formula I-A-D1.2, wherein R^{L}, R^{V}, R^{W} are H, R^{E} is CH₃, R^{X} is CH₃, and m is 2,
Table 4. Compounds of formula I-A-D1.2, wherein R^{L}, R^{V}, R^{W} are H, R^{E} is CH₃, R^{X} is C₂H₅, and m is 2
Table 5. Compounds of formula I-A-D1.2, wherein R^{L}, R^{W}, R^{E} are H, R^{V} is CF₃, R^{X} is C₂H₅, and m is 2.
Table 6. Compounds of formula I-A-D1.2, wherein R^{L}, R^{V}, R^{W} are H, R^{E} is CH₃, R^{V} is CF₃, R^{X} is C₂H₅, and m is 2.
Table 7. Compounds of formula I-A-D1.2, wherein R^{L}, R^{W}, R^{E} are H, R^{V} is OCF₃, R^{X} is C₂H₅, and m is 2.
Table 8. Compounds of formula I-A-D1.2, wherein R^{L}, R^{V}, R^{W} are H, R^{E} is CH₃, R^{V} is OCF₃, R^{X} is C₂H₅, and m is 2.
Table 9. Compounds of formula I-A-D3.2, wherein R^{L}, R^{V}, R^{W}, R^{E} are H, R^{X} is C₂H₅, and m is 2.
Table 10. Compounds of formula I-A-D3.2, wherein R^{L}, R^{V}, R^{W} are H, R^{E} is CH₃, R^{X} is C₂H₅, and m is 2
Table 11. Compounds of formula I-A-D3.2, wherein R^{L}, R^{W}, R^{E} are H, R^{V} is CF₃, R^{X} is C₂H₅, and m is 2.
Table 12. Compounds of formula I-A-D3.2, wherein R^{L}, R^{V}, R^{W} are H, R^{E} is CH₃, R^{V} is CF₃, R^{X} is C₂H₅, and m is 2.
Table 13. Compounds of formula I-A-D3.2, wherein R^{L}, R^{W}, R^{E} are H, R^{V} is OCF₃, R^{X} is C₂H₅, and m is 2.
Table 14. Compounds of formula I-A-D3.2, wherein R^{L}, R^{V}, R^{W} are H, R^{E} is CH₃, R^{V} is OCF₃, R^{X} is C₂H₅, and m is 2.
Table 15. Compounds of formula I-A-D8.2, wherein R^{L}, R^{V}, R^{W}, R^{E} are H, R^{X} is C₂H₅, and m is 2.
Table 16. Compounds of formula I-A-D8.2, wherein R^{L}, R^{V}, R^{W} are H, R^{E} is CH₃, R^{X} is C₂H₅, and m is 2
Table 17. Compounds of formula I-A-D8.2, wherein R^{L}, R^{W}, R^{E} are H, R^{V} is CF₃, R^{X} is C₂H₅, and m is 2.
Table 18. Compounds of formula I-A-D8.2, wherein R^{L}, R^{V}, R^{W} are H, R^{E} is CH₃, R^{V} is CF₃, R^{X} is C₂H₅, and m is 2.
Table 19. Compounds of formula I-A-D8.2, wherein R^{L}, R^{W}, R^{E} are H, R^{V} is OCF₃, R^{X} is C₂H₅, and m is 2.
Table 20. Compounds of formula I-A-D8.2, wherein R^{L}, R^{V}, R^{W} are H, R^{E} is CH₃, R^{V} is OCF₃, R^{X} is C₂H₅, and m is 2.
Table 21. Compounds of formula I-A-D50.2, wherein R^{L}, R^{V}, R^{W}, R^{E} are H, R^{X} is C₂H₅, and m is 2.
Table 22. Compounds of formula I-A-D50.2, wherein R^{L}, R^{V}, R^{W} are H, R^{E} is CH₃, R^{X} is C₂H₅, and m is 2
Table 23. Compounds of formula I-A-D50.2, wherein R^{L}, R^{W}, R^{E} are H, R^{V} is CF₃, R^{X} is C₂H₅, and m is 2.
Table 24. Compounds of formula I-A-D50.2, wherein R^{L}, R^{V}, R^{W} are H, R^{E} is CH₃, R^{V} is CF₃, R^{X} is C₂H₅, and m is 2.
Table 25. Compounds of formula I-A-D50.2, wherein R^{L}, R^{W}, R^{E} are H, R^{V} is OCF₃, R^{X} is C₂H₅, and m is 2.
Table 26. Compounds of formula I-A-D50.2, wherein R^{L}, R^{V}, R^{W} are H, R^{E} is CH₃, R^{V} is OCF₃, R^{X} is C₂H₅, and m is 2.
Table 27. Compounds of formula I-A-D1.3, wherein R^{L}, R^{V}, R^{W}, R^{E} are H, R^{X} is C₂H₅, and m is 2.
Table 28. Compounds of formula I-A-D1.3, wherein R^{L}, R^{V}, R^{W} are H, R^{E} is CH₃, R^{X} is C₂H₅, and m is 2
Table 29. Compounds of formula I-A-D1.3, wherein R^{L}, R^{W}, R^{E} are H, R^{V} is CF₃, R^{X} is C₂H₅, and m is 2.
Table 30. Compounds of formula I-A-D1.3, wherein R^{L}, R^{V}, R^{W} are H, R^{E} is CH₃, R^{V} is CF₃, R^{X} is C₂H₅, and m is 2.
Table 31. Compounds of formula I-A-D1.3, wherein R^{L}, R^{W}, R^{E} are H, R^{V} is OCF₃, R^{X} is C₂H₅, and m is 2.
Table 32. Compounds of formula I-A-D1.3, wherein R^{L}, R^{V}, R^{W} are H, R^{E} is CH₃, R^{V} is OCF₃, R^{X} is C₂H₅, and m is 2.
Table 33. Compounds of formula I-A-D3-3, wherein R^{L}, R^{V}, R^{W}, R^{E} are H, R^{X} is C₂H₅, and m is 2.
Table 34. Compounds of formula I-A-D3-3, wherein R^{L}, R^{V}, R^{W} are H, R^{E} is CH₃, R^{X} is C₂H₅, and m is 2
Table 35. Compounds of formula I-A-D3-3, wherein R^{L}, R^{W}, R^{E} are H, R^{V} is CF₃, R^{X} is C₂H₅, and m is 2.
Table 36. Compounds of formula I-A-D3-3, wherein R^{L}, R^{V}, R^{W} are H, R^{E} is CH₃, R^{V} is CF₃, R^{X} is C₂H₅, and m is 2.
Table 37. Compounds of formula I-A-D3-3, wherein R^{L}, R^{W}, R^{E} are H, R^{V} is OCF₃, R^{X} is C₂H₅, and m is 2.
Table 38. Compounds of formula I-A-D3-3, wherein R^{L}, R^{V}, R^{W} are H, R^{E} is CH₃, R^{V} is OCF₃, R^{X} is C₂H₅, and m is 2.
Table 39. Compounds of formula I-A-D8.3, wherein R^{L}, R^{V}, R^{W}, R^{E} are H, R^{X} is C₂H₅, and m is 2.
Table 40. Compounds of formula I-A-D8.3, wherein R^{L}, R^{V}, R^{W} are H, R^{E} is CH₃, R^{X} is C₂H₅, and m is 2
Table 41. Compounds of formula I-A-D8.3, wherein R^{L}, R^{W}, R^{E} are H, R^{V} is CF₃, R^{X} is C₂H₅, and m is 2.
Table 42. Compounds of formula I-A-D8.3, wherein R^{L}, R^{V}, R^{W} are H, R^{E} is CH₃, R^{V} is CF₃, R^{X} is C₂H₅, and m is 2.
Table 43. Compounds of formula I-A-D8.3, wherein R^{L}, R^{W}, R^{E} are H, R^{V} is OCF₃, R^{X} is C₂H₅, and m is 2.
Table 44. Compounds of formula I-A-D8.3, wherein R^{L}, R^{V}, R^{W} are H, R^{E} is CH₃, R^{V} is OCF₃, R^{X} is C₂H₅, and m is 2.
Table 45. Compounds of formula I-A-D50.3, wherein R^{L}, R^{V}, R^{W}, R^{E} are H, R^{X} is C₂H₅, and m is 2.
Table 46. Compounds of formula I-A-D50.3, wherein R^{L}, R^{V}, R^{W} are H, R^{E} is CH₃, R^{X} is C₂H₅, and m is 2
Table 47. Compounds of formula I-A-D50.3, wherein R^{L}, R^{W}, R^{E} are H, R^{V} is CF₃, R^{X} is C₂H₅, and m is 2.
Table 48. Compounds of formula I-A-D50.3, wherein R^{L}, R^{V}, R^{W} are H, R^{E} is CH₃, R^{V} is CF₃, R^{X} is C₂H₅, and m is 2.
Table 49. Compounds of formula I-A-D50.3, wherein R^{L}, R^{W}, R^{E} are H, R^{V} is OCF₃, R^{X} is C₂H₅, and m is 2.
Table 50. Compounds of formula I-A-D50.3, wherein R^{L}, R^{V}, R^{W} are H, R^{E} is CH₃, R^{V} is OCF₃, R^{X} is C₂H₅, and m is 2.
Table 51. Compounds of formula I-C-D1.2, wherein R^{L}, R^{M}, R^{V}, R^{E} are H, R^{X} is C₂H₅, and m is 2.
Table 52. Compounds of formula I-C-D1.2, wherein R^{L}, R^{M}, and R^{V} are H, R^{E} is CH₃, R^{X} is C₂H₅, and m is 2
Table 53. Compounds of formula I-C-D1.2, wherein R^{L}, R^{M}, and R^{E} are H, R^{V} is CF₃, R^{X} is C₂H₅, and m is 2.
Table 54. Compounds of formula I-C-D1.2, wherein R^{L}, R^{M}, and R^{V} are H, R^{E} is CH₃, R^{V} is CF₃, R^{X} is C₂H₅, and m is 2.
Table 55. Compounds of formula I-C-D1.2, wherein R^{L}, R^{M}, R^{E} are H, R^{V} is OCF₃, R^{X} is C₂H₅, and m is 2.
Table 56. Compounds of formula I-C-D1.2, wherein R^{L}, R^{M}, R^{V} are H, R^{E} is CH₃, R^{V} is OCF₃, R^{X} is C₂H₅, and m is 2.
Table 57. Compounds of formula I-C-D1.2, wherein R^{L}, R^{V}, R^{E} are H, R^{M} is CF₃, R^{X} is C₂H₅, and m is 2.
Table 58. Compounds of formula I-C-D1.2, wherein R^{L}, R^{V} are H, R^{M} is CF₃, R^{E} is CH₃, R^{X} is C₂H₅, and m is 2
Table 59. Compounds of formula I-C-D1.2, wherein R^{L}, R^{E} are H, R^{M} is CF₃, R^{V} is CF₃, R^{X} is C₂H₅, and m is 2.
Table 60. Compounds of formula I-C-D1.2, wherein R^{L}, R^{V} are H, R^{E} is CH₃, R^{M} is CF₃, R^{V} is CF₃, R^{X} is C₂H₅, and m is 2.
Table 61. Compounds of formula I-C-D1.2, wherein R^{L}, R^{E} are H, R^{M} is CF₃, R^{V} is OCF₃, R^{X} is C₂H₅, and m is 2.
Table 62. Compounds of formula I-C-D1.2, wherein R^{L}, R^{V} are H, R^{E} is CH₃, R^{M} is CF₃, R^{V} is OCF₃, R^{X} is C₂H₅, and m is 2.
Table 63. Compounds of formula I-C-D1.2, wherein R^{L}, R^{V}, R^{E} are H, R^{M} is OCF₃, R^{X} is C₂H₅, and m is 2.
Table 64. Compounds of formula I-C-D1.2, wherein R^{L}, R^{V} are H, R^{M} is OCF₃, R^{E} is CH₃, R^{X} is C₂H₅, and m is 2
Table 65. Compounds of formula I-C-D1.2, wherein R^{L}, R^{E} are H, R^{M} is OCF₃, R^{V} is CF₃, R^{X} is C₂H₅, and m is 2.
Table 66. Compounds of formula I-C-D1.2, wherein R^{L}, R^{V} are H, R^{E} is CH₃, R^{M} is OCF₃, R^{V} is CF₃, R^{X} is C₂H₅, and m is 2.
Table 67. Compounds of formula I-C-D1.2, wherein R^{L}, R^{E} are H, R^{M} is OCF₃, R^{V} is OCF₃, R^{X} is C₂H₅, and m is 2.
Table 68. Compounds of formula I-C-D1.2, wherein R^{L}, R^{V} are H, R^{E} is CH₃, R^{M} is OCF₃, R^{V} is OCF₃, R^{X} is C₂H₅, and m is 2.
Table 69. Compounds of formula I-C-D3.2, wherein R^{L}, R^{M}, R^{V}, R^{E} are H, R^{X} is C₂H₅, and m is 2.
Table 70. Compounds of formula I-C-D3.2, wherein R^{L}, R^{M}, R^{V} are H, R^{E} is CH₃, R^{X} is C₂H₅, and m is 2
Table 71. Compounds of formula I-C-D3.2, wherein R^{L}, R^{M}, R^{E} are H, R^{V} is CF₃, R^{X} is C₂H₅, and m is 2.
Table 72. Compounds of formula I-C-D3.2, wherein R^{L}, R^{M}, R^{V} are H, R^{E} is CH₃, R^{V} is CF₃, R^{X} is C₂H₅, and m is 2.
Table 73. Compounds of formula I-C-D3.2, wherein R^{L}, R^{M}, R^{E} are H, R^{V} is OCF₃, R^{X} is C₂H₅, and m is 2.
Table 74. Compounds of formula I-C-D3.2, wherein R^{L}, R^{M}, R^{V} are H, R^{E} is CH₃, R^{V} is OCF₃, R^{X} is C₂H₅, and m is 2.
Table 75. Compounds of formula I-C-D3.2, wherein R^{L}, R^{V}, R^{E} are H, R^{M} is CF₃, R^{X} is C₂H₅, and m is 2.
Table 76. Compounds of formula I-C-D3.2, wherein R^{L}, R^{V} are H, R^{M} is CF₃, R^{E} is CH₃, R^{X} is C₂H₅, and m is 2
Table 77. Compounds of formula I-C-D3.2, wherein R^{L}, R^{E} are H, R^{M} is CF₃, R^{V} is CF₃, R^{X} is C₂H₅, and m is 2.
Table 78. Compounds of formula I-C-D3.2, wherein R^{L}, R^{V} are H, R^{E} is CH₃, R^{M} is CF₃, R^{V} is CF₃, R^{X} is C₂H₅, and m is 2.
Table 79. Compounds of formula I-C-D3.2, wherein R^{L}, R^{E} are H, R^{M} is CF₃, R^{V} is OCF₃, R^{X} is C₂H₅, and m is 2.
Table 80. Compounds of formula I-C-D3.2, wherein R^{L}, R^{V} are H, R^{E} is CH₃, R^{M} is CF₃, R^{V} is OCF₃, R^{X} is C₂H₅, and m is 2.
Table 81. Compounds of formula I-C-D3.2, wherein R^{L}, R^{V}, R^{E} are H, R^{M} is OCF₃, R^{X} is C₂H₅, and m is 2.
Table 82. Compounds of formula I-C-D3.2, wherein R^{L}, R^{V} are H, R^{M} is OCF₃, R^{E} is CH₃, R^{X} is C₂H₅, and m is 2
Table 83. Compounds of formula I-C-D3.2, wherein R^{L}, R^{E} are H, R^{M} is OCF₃, R^{V} is CF₃, R^{X} is C₂H₅, and m is 2.
Table 84. Compounds of formula I-C-D3.2, wherein R^{L}, R^{V} are H, R^{E} is CH₃, R^{M} is OCF₃, R^{V} is CF₃, R^{X} is C₂H₅, and m is 2.
Table 85. Compounds of formula I-C-D3.2, wherein R^{L}, R^{E} are H, R^{M} is OCF₃, R^{V} is OCF₃, R^{X} is C₂H₅, and m is 2.
Table 86. Compounds of formula I-C-D3.2, wherein R^{L}, R^{V} are H, R^{E} is CH₃, R^{M} is OCF₃, R^{V} is OCF₃, R^{X} is C₂H₅, and m is 2.
Table 87. Compounds of formula I-C-D8.2, wherein R^{L}, R^{M}, R^{V} R^{E} are H, R^{X} is C₂H₅, and m is 2.
Table 88. Compounds of formula I-C-D8.2, wherein R^{L}, R^{M}, R^{V} are H, R^{E} is CH₃, R^{X} is C₂H₅, and m is 2
Table 89. Compounds of formula I-C-D8.2, wherein R^{L}, R^{M}, R^{E} are H, R^{V} is CF₃, R^{X} is C₂H₅, and m is 2.
Table 90. Compounds of formula I-C-D8.2, wherein R^{L}, R^{M}, R^{V} are H, R^{E} is CH₃, R^{V} is CF₃, R^{X} is C₂H₅, and m is 2.
Table 91. Compounds of formula I-C-D8.2, wherein R^{L}, R^{M}, R^{E} are H, R^{V} is OCF₃, R^{X} is C₂H₅, and m is 2.
Table 92. Compounds of formula I-C-D8.2, wherein R^{L}, R^{M}, R^{V} are H, R^{E} is CH₃, R^{V} is OCF₃, R^{X} is C₂H₅, and m is 2.
Table 93. Compounds of formula I-C-D8.2, wherein R^{L}, R^{V}, R^{E} are H, R^{M} is CF₃, R^{X} is C₂H₅, and m is 2.
Table 94. Compounds of formula I-C-D8.2, wherein R^{L}, R^{V} are H, R^{M} is CF₃, R^{E} is CH₃, R^{X} is C₂H₅, and m is 2
Table 95. Compounds of formula I-C-D8.2, wherein R^{L}, R^{E} are H, R^{M} is CF₃, R^{V} is CF₃, R^{X} is C₂H₅, and m is 2.
Table 96. Compounds of formula I-C-D8.2, wherein R^{L}, R^{V} are H, R^{E} is CH₃, R^{M} is CF₃, R^{V} is CF₃, R^{X} is C₂H₅, and m is 2.
Table 97. Compounds of formula I-C-D8.2, wherein R^{L}, R^{E} are H, R^{M} is CF₃, R^{V} is OCF₃, R^{X} is C₂H₅, and m is 2.
Table 98. Compounds of formula I-C-D8.2, wherein R^{L}, R^{V} are H, R^{E} is CH₃, R^{M} is CF₃, R^{V} is OCF₃, R^{X} is C₂H₅, and m is 2.
Table 99. Compounds of formula I-C-D8.2, wherein R^{L}, R^{V}, R^{E} are H, R^{M} is OCF₃, R^{X} is C₂H₅, and m is 2.
Table 100. Compounds of formula I-C-D8.2, wherein R^{L}, R^{V} are H, R^{M} is OCF₃, R^{E} is CH₃, R^{X} is C₂H₅, and m is 2
Table 101. Compounds of formula I-C-D8.2, wherein R^{L}, R^{E} are H, R^{M} is OCF₃, R^{V} is CF₃, R^{X} is C₂H₅, and m is 2.
Table 102. Compounds of formula I-C-D8.2, wherein R^{L}, R^{V} are H, R^{E} is CH₃, R^{M} is OCF₃, R^{V} is CF₃, R^{X} is C₂H₅, and m is 2.
Table 103. Compounds of formula I-C-D8.2, wherein R^{L}, R^{E} are H, R^{M} is OCF₃, R^{V} is OCF₃, R^{X} is C₂H₅, and m is 2.
Table 104. Compounds of formula I-C-D8.2, wherein R^{L}, R^{V} are H, R^{E} is CH₃, R^{M} is OCF₃, R^{V} is OCF₃, R^{X} is C₂H₅, and m is 2.
Table 105. Compounds of formula I-C-D50.2, wherein R^{L}, R^{M}, R^{V}, R^{E} are H, R^{X} is C₂H₅, and m is 2.
Table 106. Compounds of formula I-C-D50.2, wherein R^{L}, R^{M}, R^{V} are H, R^{E} is CH₃, R^{X} is C₂H₅, and m is 2
Table 107. Compounds of formula I-C-D50.2, wherein R^{L}, R^{M}, R^{E} are H, R^{V} is CF₃, R^{X} is C₂H₅, and m is 2.
Table 108. Compounds of formula I-C-D50.2, wherein R^{L}, R^{M}, R^{V} are H, R^{E} is CH₃, R^{V} is CF₃, R^{X} is C₂H₅, and m is 2.
Table 109. Compounds of formula I-C-D50.2, wherein R^{L}, R^{M}, R^{E} are H, R^{V} is OCF₃, R^{X} is C₂H₅, and m is 2.
Table 110. Compounds of formula I-C-D50.2, wherein R^{L}, R^{M}, R^{V} are H, R^{E} is CH₃, R^{V} is OCF₃, R^{X} is C₂H₅, and m is 2.
Table 111. Compounds of formula I-C-D50.2, wherein R^{L}, R^{V}, R^{E} are H, R^{M} is CF₃, R^{X} is C₂H₅, and m is 2.
Table 112. Compounds of formula I-C-D50.2, wherein R^{L}, R^{V} are H, R^{M} is CF₃, R^{E} is CH₃, R^{X} is C₂H₅, and m is 2
Table 113. Compounds of formula I-C-D50.2, wherein R^{L}, R^{E} are H, R^{M} is CF₃, R^{V} is CF₃, R^{X} is C₂H₅, and m is 2.
Table 114. Compounds of formula I-C-D50.2, wherein R^{L}, R^{V} are H, R^{E} is CH₃, R^{M} is CF₃, R^{V} is CF₃, R^{X} is C₂H₅, and m is 2.
Table 115. Compounds of formula I-C-D50.2, wherein R^{L}, R^{E} are H, R^{M} is CF₃, R^{V} is OCF₃, R^{X} is C₂H₅, and m is 2.
Table 116. Compounds of formula I-C-D50.2, wherein R^{L}, R^{V} are H, R^{E} is CH₃, R^{M} is CF₃, R^{V} is OCF₃, R^{X} is C₂H₅, and m is 2.
Table 117. Compounds of formula I-C-D50.2, wherein R^{L}, R^{V}, R^{E} are H, R^{M} is OCF₃, R^{X} is C₂H₅, and m is 2.
Table 118. Compounds of formula I-C-D50.2, wherein R^{L}, R^{V} are H, R^{M} is OCF₃, R^{E} is CH₃, R^{X} is C₂H₅, and m is 2
Table 119. Compounds of formula I-C-D50.2, wherein R^{L}, R^{E} are H, R^{M} is OCF₃, R^{V} is CF₃, R^{X} is C₂H₅, and m is 2.
Table 120. Compounds of formula I-C-D50.2, wherein R^{L}, R^{V} are H, R^{E} is CH₃, R^{M} is OCF₃, R^{V} is CF₃, R^{X} is C₂H₅, and m is 2.
Table 121. Compounds of formula I-C-D50.2, wherein R^{L}, R^{E} are H, R^{M} is OCF₃, R^{V} is OCF₃, R^{X} is C₂H₅, and m is 2.
Table 122. Compounds of formula I-C-D50.2, wherein R^{L}, R^{V} are H, R^{E} is CH₃, R^{M} is OCF₃, R^{V} is OCF₃, R^{X} is C₂H₅, and m is 2.
Table 123. Compounds of formula I-C-D1.3, wherein R^{L}, R^{M}, R^{V}, R^{E} are H, R^{X} is C₂H₅, and m is 2.
Table 124. Compounds of formula I-C-D1.3, wherein R^{L}, R^{M}, R^{V} are H, R^{E} is CH₃, R^{X} is C₂H₅, and m is 2
Table 125. Compounds of formula I-C-D1.3, wherein R^{L}, R^{M}, R^{E} are H, R^{V} is CF₃, R^{X} is C₂H₅, and m is 2.
Table 126. Compounds of formula I-C-D1.3, wherein R^{L}, R^{M}, R^{V} are H, R^{E} is CH₃, R^{V} is CF₃, R^{X} is C₂H₅, and m is 2.
Table 127. Compounds of formula I-C-D1.3, wherein R^{L}, R^{M}, R^{E} are H, R^{V} is OCF₃, R^{X} is C₂H₅, and m is 2.
Table 128. Compounds of formula I-C-D1.3, wherein R^{L}, R^{M}, R^{V} are H, R^{E} is CH₃, R^{V} is OCF₃, R^{X} is C₂H₅, and m is 2.
Table 129. Compounds of formula I-C-D1.3, wherein R^{L}, R^{V}, R^{E} are H, R^{M} is CF₃, R^{X} is C₂H₅, and m is 2.
Table 130. Compounds of formula I-C-D1.3, wherein R^{L}, R^{V} are H, R^{M} is CF₃, R^{E} is CH₃, R^{X} is C₂H₅, and m is 2
Table 131. Compounds of formula I-C-D1.3, wherein R^{L}, R^{E} are H, R^{M} is CF₃, R^{V} is CF₃, R^{X} is C₂H₅, and m is 2.
Table 132. Compounds of formula I-C-D1.3, wherein R^{L}, R^{V} are H, R^{E} is CH₃, R^{M} is CF₃, R^{V} is CF₃, R^{X} is C₂H₅, and m is 2.
Table 133. Compounds of formula I-C-D1.3, wherein R^{L}, R^{E} are H, R^{M} is CF₃, R^{V} is OCF₃, R^{X} is C₂H₅, and m is 2.
Table 134. Compounds of formula I-C-D1.3, wherein R^{L}, R^{V} are H, R^{E} is CH₃, R^{M} is CF₃, R^{V} is OCF₃, R^{X} is C₂H₅, and m is 2.
Table 135. Compounds of formula I-C-D1.3, wherein R^{L}, R^{V}, R^{E} are H, R^{M} is OCF₃, R^{X} is C₂H₅, and m is 2.
Table 136. Compounds of formula I-C-D1.3, wherein R^{L}, R^{V} are H, R^{M} is OCF₃, R^{E} is CH₃, R^{X} is C₂H₅, and m is 2
Table 137. Compounds of formula I-C-D1.3, wherein R^{L}, R^{E} are H, R^{M} is OCF₃, R^{V} is CF₃, R^{X} is C₂H₅, and m is 2.
Table 138. Compounds of formula I-C-D1.3, wherein R^{L}, R^{V} are H, R^{E} is CH₃, R^{M} is OCF₃, R^{V} is CF₃, R^{X} is C₂H₅, and m is 2.
Table 139. Compounds of formula I-C-D1.3, wherein R^{L}, R^{E} are H, R^{M} is OCF₃, R^{V} is OCF₃, R^{X} is C₂H₅, and m is 2.
Table 140. Compounds of formula I-C-D1.3, wherein R^{L}, R^{V} are H, R^{E} is CH₃, R^{M} is OCF₃, R^{V} is OCF₃, R^{X} is C₂H₅, and m is 2.
Table 141. Compounds of formula I-C-D3-3, wherein R^{L}, R^{M}, R^{V}, R^{E} are H, R^{X} is C₂H₅, and m is 2.
Table 142. Compounds of formula I-C-D3-3, wherein R^{L}, R^{M}, R^{V} are H, R^{E} is CH₃, R^{X} is C₂H₅, and m is 2
Table 143. Compounds of formula I-C-D3-3, wherein R^{L}, R^{M}, R^{E} are H, R^{V} is CF₃, R^{X} is C₂H₅, and m is 2.
Table 144. Compounds of formula I-C-D3-3, wherein R^{L}, R^{M}, R^{V} are H, R^{E} is CH₃, R^{V} is CF₃, R^{X} is C₂H₅, and m is 2.
Table 145. Compounds of formula I-C-D3-3, wherein R^{L}, R^{M}, R^{E} are H, R^{V} is OCF₃, R^{X} is C₂H₅, and m is 2.
Table 146. Compounds of formula I-C-D3-3, wherein R^{L}, R^{M}, R^{V} are H, R^{E} is CH₃, R^{V} is OCF₃, R^{X} is C₂H₅, and m is 2.
Table 147. Compounds of formula I-C-D3-3, wherein R^{L}, R^{V}, R^{E} are H, R^{M} is CF₃, R^{X} is C₂H₅, and m is 2.
Table 148. Compounds of formula I-C-D3-3, wherein R^{L}, R^{V} are H, R^{M} is CF₃, R^{E} is CH₃, R^{X} is C₂H₅, and m is 2
Table 149. Compounds of formula I-C-D3-3, wherein R^{L}, R^{E} are H, R^{M} is CF₃, R^{V} is CF₃, R^{X} is C₂H₅, and m is 2.
Table 150. Compounds of formula I-C-D3-3, wherein R^{L}, R^{V} are H, R^{E} is CH₃, R^{M} is CF₃, R^{V} is CF₃, R^{X} is C₂H₅, and m is 2.
Table 151. Compounds of formula I-C-D3-3, wherein R^{L}, R^{E} are H, R^{M} is CF₃, R^{V} is OCF₃, R^{X} is C₂H₅, and m is 2.
Table 152. Compounds of formula I-C-D3-3, wherein R^{L}, R^{V} are H, R^{E} is CH₃, R^{M} is CF₃, R^{V} is OCF₃, R^{X} is C₂H₅, and m is 2.
Table 153. Compounds of formula I-C-D3-3, wherein R^{L}, R^{V}, R^{E} are H, R^{M} is OCF₃, R^{X} is C₂H₅, and m is 2.
Table 154. Compounds of formula I-C-D3-3, wherein R^{L}, R^{V} are H, R^{M} is OCF₃, R^{E} is CH₃, R^{X} is C₂H₅, and m is 2
Table 155. Compounds of formula I-C-D3-3, wherein R^{L}, R^{E} are H, R^{M} is OCF₃, R^{V} is CF₃, R^{X} is C₂H₅, and m is 2.
Table 156. Compounds of formula I-C-D3-3, wherein R^{L}, R^{V} are H, R^{E} is CH₃, R^{M} is OCF₃, R^{V} is CF₃, R^{X} is C₂H₅, and m is 2.
Table 157. Compounds of formula I-C-D3-3, wherein R^{L}, R^{E} are H, R^{M} is OCF₃, R^{V} is OCF₃, R^{X} is C₂H₅, and m is 2.
Table 158. Compounds of formula I-C-D3-3, wherein R^{L}, R^{V} are H, R^{E} is CH₃, R^{M} is OCF₃, R^{V} is OCF₃, R^{X} is C₂H₅, and m is 2.
Table 159. Compounds of formula I-C-D8.3, wherein R^{L}, R^{M}, R^{V}, R^{E} are H, R^{X} is C₂H₅, and m is 2.
Table 160. Compounds of formula I-C-D8.3, wherein R^{L}, R^{M}, R^{V} are H, R^{E} is CH₃, R^{X} is C₂H₅, and m is 2
Table 161. Compounds of formula I-C-D8.3, wherein R^{L}, R^{M}, R^{E} are H, R^{V} is CF₃, R^{X} is C₂H₅, and m is 2.
Table 162. Compounds of formula I-C-D8.3, wherein R^{L}, R^{M}, R^{V} are H, R^{E} is CH₃, R^{V} is CF₃, R^{X} is C₂H₅, and m is 2.
Table 163. Compounds of formula I-C-D8.3, wherein R^{L}, R^{M}, R^{E} are H, R^{V} is OCF₃, R^{X} is C₂H₅, and m is 2.
Table 164. Compounds of formula I-C-D8.3, wherein R^{L}, R^{M}, R^{V} are H, R^{E} is CH₃, R^{V} is OCF₃, R^{X} is C₂H₅, and m is 2.
Table 165. Compounds of formula I-C-D8.3, wherein R^{L}, R^{V}, R^{E} are H, R^{M} is CF₃, R^{X} is C₂H₅, and m is 2.
Table 166. Compounds of formula I-C-D8.3, wherein R^{L}, R^{V} are H, R^{M} is CF₃, R^{E} is CH₃, R^{X} is C₂H₅, and m is 2
Table 167. Compounds of formula I-C-D8.3, wherein R^{L}, R^{E} are H, R^{M} is CF₃, R^{V} is CF₃, R^{X} is C₂H₅, and m is 2.
Table 168. Compounds of formula I-C-D8.3, wherein R^{L}, R^{V} are H, R^{E} is CH₃, R^{M} is CF₃, R^{V} is CF₃, R^{X} is C₂H₅, and m is 2.
Table 169. Compounds of formula I-C-D8.3, wherein R^{L}, R^{E} are H, R^{M} is CF₃, R^{V} is OCF₃, R^{X} is C₂H₅, and m is 2.
Table 170. Compounds of formula I-C-D8.3, wherein R^{L}, R^{V} are H, R^{E} is CH₃, R^{M} is CF₃, R^{V} is OCF₃, R^{X} is C₂H₅, and m is 2.
Table 171. Compounds of formula I-C-D8.3, wherein R^{L}, R^{V}, R^{E} are H, R^{M} is OCF₃, R^{X} is C₂H₅, and m is 2.
Table 172. Compounds of formula I-C-D8.3, wherein R^{L}, R^{V} are H, R^{M} is OCF₃, R^{E} is CH₃, R^{X} is C₂H₅, and m is 2
Table 173. Compounds of formula I-C-D8.3, wherein R^{L}, R^{E} are H, R^{M} is OCF₃, R^{V} is CF₃, R^{X} is C₂H₅, and m is 2.
Table 174. Compounds of formula I-C-D8.3, wherein R^{L}, R^{V} are H, R^{E} is CH₃, R^{M} is OCF₃, R^{V} is CF₃, R^{X} is C₂H₅, and m is 2.
Table 175. Compounds of formula I-C-D8.3, wherein R^{L}, R^{E} are H, R^{M} is OCF₃, R^{V} is OCF₃, R^{X} is C₂H₅, and m is 2.
Table 176. Compounds of formula I-C-D8.3, wherein R^{L}, R^{V} are H, R^{E} is CH₃, R^{M} is OCF₃, R^{V} is OCF₃, R^{X} is C₂H₅, and m is 2.
Table 177. Compounds of formula I-C-D50.3, wherein R^{L}, R^{M}, R^{V}, R^{E} are H, R^{X} is C₂H₅, and m is 2.
Table 178. Compounds of formula I-C-D50.3, wherein R^{L}, R^{M}, R^{V} are H, R^{E} is CH₃, R^{X} is C₂H₅, and m is 2
Table 179. Compounds of formula I-C-D50.3, wherein R^{L}, R^{M}, R^{E} are H, R^{V} is CF₃, R^{X} is C₂H₅, and m is 2.
Table 180. Compounds of formula I-C-D50.3, wherein R^{L}, R^{M}, R^{V} are H, R^{E} is CH₃, R^{V} is CF₃, R^{X} is C₂H₅, and m is 2.
Table 181. Compounds of formula I-C-D50.3, wherein R^{L}, R^{M}, R^{E} are H, R^{V} is OCF₃, R^{X} is C₂H₅, and m is 2.
Table 182. Compounds of formula I-C-D50.3, wherein R^{L}, R^{M}, R^{V} are H, R^{E} is CH₃, R^{V} is OCF₃, R^{X} is C₂H₅, and m is 2.
Table 183. Compounds of formula I-C-D50.3, wherein R^{L}, R^{V}, R^{E} are H, R^{M} is CF₃, R^{X} is C₂H₅, and m is 2.
Table 184. Compounds of formula I-C-D50.3, wherein R^{L}, R^{V} are H, R^{M} is CF₃, R^{E} is CH₃, R^{X} is C₂H₅, and m is 2
Table 185. Compounds of formula I-C-D50.3, wherein R^{L}, R^{E} are H, R^{M} is CF₃, R^{V} is CF₃, R^{X} is C₂H₅, and m is 2.
Table 186. Compounds of formula I-C-D50.3, wherein R^{L}, R^{V} are H, R^{E} is CH₃, R^{M} is CF₃, R^{V} is CF₃, R^{X} is C₂H₅, and m is 2.
Table 187. Compounds of formula I-C-D50.3, wherein R^{L}, R^{E} are H, R^{M} is CF₃, R^{V} is OCF₃, R^{X} is C₂H₅, and m is 2.
Table 188. Compounds of formula I-C-D50.3, wherein R^{L}, R^{V} are H, R^{E} is CH₃, R^{M} is CF₃, R^{V} is OCF₃, R^{X} is C₂H₅, and m is 2.
Table 189. Compounds of formula I-C-D50.3, wherein R^{L}, R^{V}, R^{E} are H, R^{M} is OCF₃, R^{X} is C₂H₅, and m is 2.
Table 190. Compounds of formula I-C-D50.3, wherein R^{L}, R^{V} are H, R^{M} is OCF₃, R^{E} is CH₃, is C₂H₅, and m is 2
Table 191. Compounds of formula I-C-D50.3, wherein R^{L}, R^{E} are H, R^{M} is OCF₃, R^{V} is CF₃, R^{X} is C₂H₅, and m is 2.
Table 192. Compounds of formula I-C-D50.3, wherein R^{L}, R^{V} are H, R^{E} is CH₃, R^{M} is OCF₃, R^{V} is CF₃, R^{X} is C₂H₅, and m is 2.
Table 193. Compounds of formula I-C-D50.3, wherein R^{L}, R^{E} are H, R^{M} is OCF₃, R^{V} is OCF₃, R^{X} is C₂H₅, and m is 2.
Table 194. Compounds of formula I-C-D50.3, wherein R^{L}, R^{V} are H, R^{E} is CH₃, R^{M} is OCF₃, R^{V} is OCF₃, R^{X} is C₂H₅, and m is 2.
Table 195. Compounds of formula I-D-D1.2, wherein R^{L}, R^{M}, R^{V}, R^{W}, R^{E} are H, R^{X} is C₂H₅, and m is 2.
Table 196. Compounds of formula I-D-D1.2, wherein R^{L}, R^{M}, R^{V}, R^{W} are H, R^{E} is CH₃, R^{X} is C₂H₅, and m is 2
Table 197. Compounds of formula I-D-D1.2, wherein R^{L}, R^{M}, R^{W}, R^{E} are H, R^{V} is CF₃, R^{X} is C₂H₅, and m is 2.
Table 198. Compounds of formula I-D-D1.2, wherein R^{L}, R^{M}, R^{V}, R^{W} are H, R^{E} is CH₃, R^{V} is CF₃, R^{X} is C₂H₅, and m is 2.
Table 199. Compounds of formula I-D-D1.2, wherein R^{L}, R^{M}, R^{W}, R^{E} are H, R^{V} is OCF₃, R^{X} is C₂H₅, and m is 2.
Table 200. Compounds of formula I-D-D1.2, wherein R^{L}, R^{M}, R^{V}, R^{W} are H, R^{E} is CH₃, R^{V} is OCF₃, R^{X} is C₂H₅, and m is 2.
Table 201. Compounds of formula I-D-D1.2, wherein R^{L}, R^{V}, R^{W}, R^{E} are H, R^{M} is CF₃, R^{X} is C₂H₅, and m is 2.
Table 202. Compounds of formula I-D-D1.2, wherein R^{L}, R^{V}, R^{W} are H, R^{M} is CF₃, R^{E} is CH₃, R^{X} is C₂H₅, and m is 2
Table 203. Compounds of formula I-D-D1.2, wherein R^{L}, R^{W}, R^{E} are H, R^{M} is CF₃, R^{V} is CF₃, R^{X} is C₂H₅, and m is 2.
Table 204. Compounds of formula I-D-D1.2, wherein R^{L}, R^{V}, R^{W} are H, R^{E} is CH₃, R^{M} is CF₃, R^{V} is CF₃, R^{X} is C₂H₅, and m is 2.
Table 205. Compounds of formula I-D-D1.2, wherein R^{L}, R^{W}, R^{E} are H, R^{M} is CF₃, R^{V} is OCF₃, R^{X} is C₂H₅, and m is 2.
Table 206. Compounds of formula I-D-D1.2, wherein R^{L}, R^{V}, R^{W} are H, R^{E} is CH₃, R^{M} is CF₃, R^{V} is OCF₃, R^{X} is C₂H₅, and m is 2.
Table 207. Compounds of formula I-D-D1.2, wherein R^{L}, R^{V}, R^{W}, R^{E} are H, R^{M} is OCF₃, R^{X} is C₂H₅, and m is 2.
Table 208. Compounds of formula I-D-D1.2, wherein R^{L}, R^{V}, R^{W} are H, R^{M} is OCF₃, R^{E} is CH₃, R^{X} is C₂H₅, and m is 2
Table 209. Compounds of formula I-D-D1.2, wherein R^{L}, R^{W}, R^{E} are H, R^{M} is OCF₃, R^{V} is CF₃, R^{X} is C₂H₅, and m is 2.
Table 210. Compounds of formula I-D-D1.2, wherein R^{L}, R^{V}, R^{W} are H, R^{E} is CH₃, R^{M} is OCF₃, R^{V} is CF₃, R^{X} is C₂H₅, and m is 2.
Table 211. Compounds of formula I-D-D1.2, wherein R^{L}, R^{W}, R^{E} are H, R^{M} is OCF₃, R^{V} is OCF₃, R^{X} is C₂H₅, and m is 2.
Table 212. Compounds of formula I-D-D1.2, wherein R^{L}, R^{V}, R^{W} are H, R^{E} is CH₃, R^{M} is OCF₃, R^{V} is OCF₃, R^{X} is C₂H₅, and m is 2.
Table 213. Compounds of formula I-D-D3.2, wherein R^{L}, R^{M}, R^{V}, R^{W}, R^{E} are H, R^{X} is C₂H₅, and m is 2.
Table 214. Compounds of formula I-D-D3.2, wherein R^{L}, R^{M}, R^{V}, R^{W} are H, R^{E} is CH₃, R^{X} is C₂H₅, and m is 2
Table 215. Compounds of formula I-D-D3.2, wherein R^{L}, R^{M}, R^{W}, R^{E} are H, R^{V} is CF₃, R^{X} is C₂H₅, and m is 2.
Table 216. Compounds of formula I-D-D3.2, wherein R^{L}, R^{M}, R^{V}, R^{W} are H, R^{E} is CH₃, R^{V} is CF₃, R^{X} is C₂H₅, and m is 2.
Table 217. Compounds of formula I-D-D3.2, wherein R^{L}, R^{M}, R^{W}, R^{E} are H, R^{V} is OCF₃, R^{X} is C₂H₅, and m is 2.
Table 218. Compounds of formula I-D-D3.2, wherein R^{L}, R^{M}, R^{V}, R^{W} are H, R^{E} is CH₃, R^{V} is OCF₃, R^{X} is C₂H₅, and m is 2.
Table 219. Compounds of formula I-D-D3.2, wherein R^{L}, R^{V}, R^{W}, R^{E} are H, R^{M} is CF₃, R^{X} is C₂H₅, and m is 2.
Table 220. Compounds of formula I-D-D3.2, wherein R^{L}, R^{V}, R^{W} are H, R^{M} is CF₃, R^{E} is CH₃, R^{X} is C₂H₅, and m is 2
Table 221. Compounds of formula I-D-D3.2, wherein R^{L}, R^{W}, R^{E} are H, R^{M} is CF₃, R^{V} is CF₃, R^{X} is C₂H₅, and m is 2.
Table 222. Compounds of formula I-D-D3.2, wherein R^{L}, R^{V}, R^{W} are H, R^{E} is CH₃, R^{M} is CF₃, R^{V} is CF₃, R^{X} is C₂H₅, and m is 2.
Table 223. Compounds of formula I-D-D3.2, wherein R^{L}, R^{W}, R^{E} are H, R^{M} is CF₃, R^{V} is OCF₃, R^{X} is C₂H₅, and m is 2.
Table 224. Compounds of formula I-D-D3.2, wherein R^{L}, R^{V}, R^{W} are H, R^{E} is CH₃, R^{M} is CF₃, R^{V} is OCF₃, R^{X} is C₂H₅, and m is 2.
Table 225. Compounds of formula I-D-D3.2, wherein R^{L}, R^{V}, R^{W}, R^{E} are H, R^{M} is OCF₃, R^{X} is C₂H₅, and m is 2.
Table 226. Compounds of formula I-D-D3.2, wherein R^{L}, R^{V}, R^{W} are H, R^{M} is OCF₃, R^{E} is CH₃, R^{X} is C₂H₅, and m is 2
Table 227. Compounds of formula I-D-D3.2, wherein R^{L}, R^{W}, R^{E} are H, R^{M} is OCF₃, R^{V} is CF₃, R^{X} is C₂H₅, and m is 2.
Table 228. Compounds of formula I-D-D3.2, wherein R^{L}, R^{V}, R^{W} are H, R^{E} is CH₃, R^{M} is OCF₃, R^{V} is CF₃, R^{X} is C₂H₅, and m is 2.
Table 229. Compounds of formula I-D-D3.2, wherein R^{L}, R^{W}, R^{E} are H, R^{M} is OCF₃, R^{V} is OCF₃, R^{X} is C₂H₅, and m is 2.
Table 230. Compounds of formula I-D-D3.2, wherein R^{L}, R^{V}, R^{W} are H, R^{E} is CH₃, R^{M} is OCF₃, R^{V} is OCF₃, R^{X} is C₂H₅, and m is 2.
Table 231. Compounds of formula I-D-D8.2, wherein R^{L}, R^{M}, R^{V}, R^{W}, R^{E} are H, R^{X} is C₂H₅, and m is 2.
Table 232. Compounds of formula I-D-D8.2, wherein R^{L}, R^{M}, R^{V}, R^{W} are H, R^{E} is CH₃, R^{X} is C₂H₅, and m is 2
Table 233. Compounds of formula I-D-D8.2, wherein R^{L}, R^{M}, R^{W}, R^{E} are H, R^{V} is CF₃, R^{X} is C₂H₅, and m is 2.
Table 234. Compounds of formula I-D-D8.2, wherein R^{L}, R^{M}, R^{V}, R^{W} are H, R^{E} is CH₃, R^{V} is CF₃, R^{X} is C₂H₅, and m is 2.
Table 235. Compounds of formula I-D-D8.2, wherein R^{L}, R^{M}, R^{W}, R^{E} are H, R^{V} is OCF₃, R^{X} is C₂H₅, and m is 2.
Table 236. Compounds of formula I-D-D8.2, wherein R^{L}, R^{M}, R^{V}, R^{W} are H, R^{E} is CH₃, R^{V} is OCF₃, R^{X} is C₂H₅, and m is 2.
Table 237. Compounds of formula I-D-D8.2, wherein R^{L}, R^{V}, R^{W}, R^{E} are H, R^{M} is CF₃, R^{X} is C₂H₅, and m is 2.
Table 238. Compounds of formula I-D-D8.2, wherein R^{L}, R^{V}, R^{W} are H, R^{M} is CF₃, R^{E} is CH₃, R^{X} is C₂H₅, and m is 2
Table 239. Compounds of formula I-D-D8.2, wherein R^{L}, R^{W}, R^{E} are H, R^{M} is CF₃, R^{V} is CF₃, R^{X} is C₂H₅, and m is 2.
Table 240. Compounds of formula I-D-D8.2, wherein R^{L}, R^{V}, R^{W} are H, R^{E} is CH₃, R^{M} is CF₃, R^{V} is CF₃, R^{X} is C₂H₅, and m is 2.
Table 241. Compounds of formula I-D-D8.2, wherein R^{L}, R^{W}, R^{E} are H, R^{M} is CF₃, R^{V} is OCF₃, R^{X} is C₂H₅, and m is 2.
Table 242. Compounds of formula I-D-D8.2, wherein R^{L}, R^{V}, R^{W} are H, R^{E} is CH₃, R^{M} is CF₃, R^{V} is OCF₃, R^{X} is C₂H₅, and m is 2.
Table 243. Compounds of formula I-D-D8.2, wherein R^{L}, R^{V}, R^{W}, R^{E} are H, R^{M} is OCF₃, R^{X} is C₂H₅, and m is 2.
Table 244. Compounds of formula I-D-D8.2, wherein R^{L}, R^{V}, R^{W} are H, R^{M} is OCF₃, R^{E} is CH₃, R^{X} is C₂H₅, and m is 2
Table 245. Compounds of formula I-D-D8.2, wherein R^{L}, R^{W}, R^{E} are H, R^{M} is OCF₃, R^{V} is CF₃, R^{X} is C₂H₅, and m is 2.
Table 246. Compounds of formula I-D-D8.2, wherein R^{L}, R^{V}, R^{W} are H, R^{E} is CH₃, R^{M} is OCF₃, R^{V} is CF₃, R^{X} is C₂H₅, and m is 2.
Table 247. Compounds of formula I-D-D8.2, wherein R^{L}, R^{W}, R^{E} are H, R^{M} is OCF₃, R^{V} is OCF₃, R^{X} is C₂H₅, and m is 2.
Table 248. Compounds of formula I-D-D8.2, wherein R^{L}, R^{V}, R^{W} are H, R^{E} is CH₃, R^{M} is OCF₃, R^{V} is OCF₃, R^{X} is C₂H₅, and m is 2.
Table 249. Compounds of formula I-D-D50.2, wherein R^{L}, R^{M}, R^{V}, R^{W}, R^{E} are H, R^{X} is C₂H₅, and m is 2.
Table 250. Compounds of formula I-D-D50.2, wherein R^{L}, R^{M}, R^{V}, R^{W} are H, R^{E} is CH₃, R^{X} is C₂H₅, and m is 2
Table 251. Compounds of formula I-D-D50.2, wherein R^{L}, R^{M}, R^{W}, R^{E} are H, R^{V} is CF₃, R^{X} is C₂H₅, and m is 2.
Table 252. Compounds of formula I-D-D50.2, wherein R^{L}, R^{M}, R^{V}, R^{W} are H, R^{E} is CH₃, R^{V} is CF₃, R^{X} is C₂H₅, and m is 2.
Table 253. Compounds of formula I-D-D50.2, wherein R^{L}, R^{M}, R^{W}, R^{E} are H, R^{V} is OCF₃, R^{X} is C₂H₅, and m is 2.
Table 254. Compounds of formula I-D-D50.2, wherein R^{L}, R^{M}, R^{V}, R^{W} are H, R^{E} is CH₃, R^{V} is OCF₃, R^{X} is C₂H₅, and m is 2.
Table 255. Compounds of formula I-D-D50.2, wherein R^{L}, R^{V}, R^{W}, R^{E} are H, R^{M} is CF₃, R^{X} is C₂H₅, and m is 2.
Table 256. Compounds of formula I-D-D50.2, wherein R^{L}, R^{V}, R^{W} are H, R^{M} is CF₃, R^{E} is CH₃, R^{X} is C₂H₅, and m is 2
Table 257. Compounds of formula I-D-D50.2, wherein R^{L}, R^{W}, R^{E} are H, R^{M} is CF₃, R^{V} is CF₃, R^{X} is C₂H₅, and m is 2.
Table 258. Compounds of formula I-D-D50.2, wherein R^{L}, R^{V}, R^{W} are H, R^{E} is CH₃, R^{M} is CF₃, R^{V} is CF₃, R^{X} is C₂H₅, and m is 2.
Table 259. Compounds of formula I-D-D50.2, wherein R^{L}, R^{W}, R^{E} are H, R^{M} is CF₃, R^{V} is OCF₃, R^{X} is C₂H₅, and m is 2.
Table 260. Compounds of formula I-D-D50.2, wherein R^{L}, R^{V}, R^{W} are H, R^{E} is CH₃, R^{M} is CF₃, R^{V} is OCF₃, R^{X} is C₂H₅, and m is 2.
Table 261. Compounds of formula I-D-D50.2, wherein R^{L}, R^{V}, R^{W}, R^{E} are H, R^{M} is OCF₃, R^{X} is C₂H₅, and m is 2.
Table 262. Compounds of formula I-D-D50.2, wherein R^{L}, R^{V}, R^{W} are H, R^{M} is OCF₃, R^{E} is CH₃, R^{X} is C₂H₅, and m is 2
Table 263. Compounds of formula I-D-D50.2, wherein R^{L}, R^{W}, R^{E} are H, R^{M} is OCF₃, R^{V} is CF₃, R^{X} is C₂H₅, and m is 2.
Table 264. Compounds of formula I-D-D50.2, wherein R^{L}, R^{V}, R^{W} are H, R^{E} is CH₃, R^{M} is OCF₃, R^{V} is CF₃, R^{X} is C₂H₅, and m is 2.
Table 265. Compounds of formula I-D-D50.2, wherein R^{L}, R^{W}, R^{E} are H, R^{M} is OCF₃, R^{V} is OCF₃, R^{X} is C₂H₅, and m is 2.
Table 266. Compounds of formula I-D-D50.2, wherein R^{L}, R^{V}, R^{W} are H, R^{E} is CH₃, R^{M} is OCF₃, R^{V} is OCF₃, R^{X} is C₂H₅, and m is 2.
Table 267. Compounds of formula I-D-D1.3, wherein R^{L}, R^{M}, R^{V}, R^{W}, R^{E} are H, R^{X} is C₂H₅, and m is 2.
Table 268. Compounds of formula I-D-D1.3, wherein R^{L}, R^{M}, R^{V}, R^{W} are H, R^{E} is CH₃, R^{X} is C₂H₅, and m is 2
Table 269. Compounds of formula I-D-D1.3, wherein R^{L}, R^{M}, R^{W}, R^{E} are H, R^{V} is CF₃, R^{X} is C₂H₅, and m is 2.
Table 270. Compounds of formula I-D-D1.3, wherein R^{L}, R^{M}, R^{V}, R^{W} are H, R^{E} is CH₃, R^{V} is CF₃, R^{X} is C₂H₅, and m is 2.
Table 271. Compounds of formula I-D-D1.3, wherein R^{L}, R^{M}, R^{W}, R^{E} are H, R^{V} is OCF₃, R^{X} is C₂H₅, and m is 2.
Table 272. Compounds of formula I-D-D1.3, wherein R^{L}, R^{M}, R^{V}, R^{W} are H, R^{E} is CH₃, R^{V} is OCF₃, R^{X} is C₂H₅, and m is 2.
Table 273. Compounds of formula I-D-D1.3, wherein R^{L}, R^{V}, R^{W}, R^{E} are H, R^{M} is CF₃, R^{X} is C₂H₅, and m is 2.
Table 274. Compounds of formula I-D-D1.3, wherein R^{L}, R^{V}, R^{W} are H, R^{M} is CF₃, R^{E} is CH₃, R^{X} is C₂H₅, and m is 2
Table 275. Compounds of formula I-D-D1.3, wherein R^{L}, R^{W}, R^{E} are H, R^{M} is CF₃, R^{V} is CF₃, R^{X} is C₂H₅, and m is 2.
Table 276. Compounds of formula I-D-D1.3, wherein R^{L}, R^{V}, R^{W} are H, R^{E} is CH₃, R^{M} is CF₃, R^{V} is CF₃, R^{X} is C₂H₅, and m is 2.
Table 277. Compounds of formula I-D-D1.3, wherein R^{L}, R^{W}, R^{E} are H, R^{M} is CF₃, R^{V} is OCF₃, R^{X} is C₂H₅, and m is 2.
Table 278. Compounds of formula I-D-D1.3, wherein R^{L}, R^{V}, R^{W} are H, R^{E} is CH₃, R^{M} is CF₃, R^{V} is OCF₃, R^{X} is C₂H₅, and m is 2.
Table 279. Compounds of formula I-D-D1.3, wherein R^{L}, R^{V}, R^{W}, R^{E} are H, R^{M} is OCF₃, R^{X} is C₂H₅, and m is 2.
Table 280. Compounds of formula I-D-D1.3, wherein R^{L}, R^{V}, R^{W} are H, R^{M} is OCF₃, R^{E} is CH₃, R^{X} is C₂H₅, and m is 2
Table 281. Compounds of formula I-D-D1.3, wherein R^{L}, R^{W}, R^{E} are H, R^{M} is OCF₃, R^{V} is CF₃, R^{X} is C₂H₅, and m is 2.
Table 282. Compounds of formula I-D-D1.3, wherein R^{L}, R^{V}, R^{W} are H, R^{E} is CH₃, R^{M} is OCF₃, R^{V} is CF₃, R^{X} is C₂H₅, and m is 2.
Table 283. Compounds of formula I-D-D1.3, wherein R^{L}, R^{W}, R^{E} are H, R^{M} is OCF₃, R^{V} is OCF₃, R^{X} is C₂H₅, and m is 2.
Table 284. Compounds of formula I-D-D1.3, wherein R^{L}, R^{V}, R^{W} are H, R^{E} is CH₃, R^{M} is OCF₃, R^{V} is OCF₃, R^{X} is C₂H₅, and m is 2.
Table 285. Compounds of formula I-D-D3-3, wherein R^{L}, R^{M}, R^{V}, R^{W}, R^{E} are H, R^{X} is C₂H₅, and m is 2.
Table 286. Compounds of formula I-D-D3-3, wherein R^{L}, R^{M}, R^{V}, R^{W} are H, R^{E} is CH₃, R^{X} is C₂H₅, and m is 2
Table 287. Compounds of formula I-D-D3-3, wherein R^{L}, R^{M}, R^{W}, R^{E} are H, R^{V} is CF₃, R^{X} is C₂H₅, and m is 2.
Table 288. Compounds of formula I-D-D3-3, wherein R^{L}, R^{M}, R^{V}, R^{W} are H, R^{E} is CH₃, R^{V} is CF₃, R^{X} is C₂H₅, and m is 2.
Table 289. Compounds of formula I-D-D3-3, wherein R^{L}, R^{M}, R^{W}, R^{E} are H, R^{V} is OCF₃, R^{X} is C₂H₅, and m is 2.
Table 290. Compounds of formula I-D-D3-3, wherein R^{L}, R^{M}, R^{V}, R^{W} are H, R^{E} is CH₃, R^{V} is OCF₃, R^{X} is C₂H₅, and m is 2.
Table 291. Compounds of formula I-D-D3-3, wherein R^{L}, R^{V}, R^{W}, R^{E} are H, R^{M} is CF₃, R^{X} is C₂H₅, and m is 2.
Table 292. Compounds of formula I-D-D3-3, wherein R^{L}, R^{V}, R^{W} are H, R^{M} is CF₃, R^{E} is CH₃, R^{X} is C₂H₅, and m is 2
Table 293. Compounds of formula I-D-D3-3, wherein R^{L}, R^{W}, R^{E} are H, R^{M} is CF₃, R^{V} is CF₃, R^{X} is C₂H₅, and m is 2.
Table 294. Compounds of formula I-D-D3-3, wherein R^{L}, R^{V}, R^{W} are H, R^{E} is CH₃, R^{M} is CF₃, R^{V} is CF₃, R^{X} is C₂H₅, and m is 2.
Table 295. Compounds of formula I-D-D3-3, wherein R^{L}, R^{W}, R^{E} are H, R^{M} is CF₃, R^{V} is OCF₃, R^{X} is C₂H₅, and m is 2.
Table 296. Compounds of formula I-D-D3-3, wherein R^{L}, R^{V}, R^{W} are H, R^{E} is CH₃, R^{M} is CF₃, R^{V} is OCF₃, R^{X} is C₂H₅, and m is 2.
Table 297. Compounds of formula I-D-D3-3, wherein R^{L}, R^{V}, R^{W}, R^{E} are H, R^{M} is OCF₃, R^{X} is C₂H₅, and m is 2.
Table 298. Compounds of formula I-D-D3-3, wherein R^{L}, R^{V}, R^{W} are H, R^{M} is OCF₃, R^{E} is CH₃, R^{X} is C₂H₅, and m is 2
Table 299. Compounds of formula I-D-D3-3, wherein R^{L}, R^{W}, R^{E} are H, R^{M} is OCF₃, R^{V} is CF₃, R^{X} is C₂H₅, and m is 2.
Table 300. Compounds of formula I-D-D3-3, wherein R^{L}, R^{V}, R^{W} are H, R^{E} is CH₃, R^{M} is OCF₃, R^{V} is CF₃, R^{X} is C₂H₅, and m is 2.
Table 301. Compounds of formula I-D-D3-3, wherein R^{L}, R^{W}, R^{E} are H, R^{M} is OCF₃, R^{V} is OCF₃, R^{X} is C₂H₅, and m is 2.
Table 302. Compounds of formula I-D-D3-3, wherein R^{L}, R^{V}, R^{W} are H, R^{E} is CH₃, R^{M} is OCF₃, R^{V} is OCF₃, R^{X} is C₂H₅, and m is 2.
Table 303. Compounds of formula I-D-D8.3, wherein R^{L}, R^{M}, R^{V}, R^{W}, R^{E} are H, R^{X} is C₂H₅, and m is 2.
Table 304. Compounds of formula I-D-D8.3, wherein R^{L}, R^{M}, R^{V}, R^{W} are H, R^{E} is CH₃, R^{X} is C₂H₅, and m is 2
Table 305. Compounds of formula I-D-D8.3, wherein R^{L}, R^{M}, R^{W}, R^{E} are H, R^{V} is CF₃, R^{X} is C₂H₅, and m is 2.
Table 306. Compounds of formula I-D-D8.3, wherein R^{L}, R^{M}, R^{V}, R^{W} are H, R^{E} is CH₃, R^{V} is CF₃, R^{X} is C₂H₅, and m is 2.
Table 307. Compounds of formula I-D-D8.3, wherein R^{L}, R^{M}, R^{W}, R^{E} are H, R^{V} is OCF₃, R^{X} is C₂H₅, and m is 2.
Table 308. Compounds of formula I-D-D8.3, wherein R^{L}, R^{M}, R^{V}, R^{W} are H, R^{E} is CH₃, R^{V} is OCF₃, R^{X} is C₂H₅, and m is 2.
Table 309. Compounds of formula I-D-D8.3, wherein R^{L}, R^{V}, R^{W}, R^{E} are H, R^{M} is CF₃, R^{X} is C₂H₅, and m is 2.
Table 310. Compounds of formula I-D-D8.3, wherein R^{L}, R^{V}, R^{W} are H, R^{M} is CF₃, R^{E} is CH₃, R^{X} is C₂H₅, and m is 2
Table 311. Compounds of formula I-D-D8.3, wherein R^{L}, R^{W}, R^{E} are H, R^{M} is CF₃, R^{V} is CF₃, R^{X} is C₂H₅, and m is 2.
Table 312. Compounds of formula I-D-D8.3, wherein R^{L}, R^{V}, R^{W} are H, R^{E} is CH₃, R^{M} is CF₃, R^{V} is CF₃, R^{X} is C₂H₅, and m is 2.
Table 313. Compounds of formula I-D-D8.3, wherein R^{L}, R^{W}, R^{E} are H, R^{M} is CF₃, R^{V} is OCF₃, R^{X} is C₂H₅, and m is 2.
Table 314. Compounds of formula I-D-D8.3, wherein R^{L}, R^{V}, R^{W} are H, R^{E} is CH₃, R^{M} is CF₃, R^{V} is OCF₃, R^{X} is C₂H₅, and m is 2.
Table 315. Compounds of formula I-D-D8.3, wherein R^{L}, R^{V}, R^{W}, R^{E} are H, R^{M} is OCF₃, R^{X} is C₂H₅, and m is 2.
Table 316. Compounds of formula I-D-D8.3, wherein R^{L}, R^{V}, R^{W} are H, R^{M} is OCF₃, R^{E} is CH₃, R^{X} is C₂H₅, and m is 2
Table 317. Compounds of formula I-D-D8.3, wherein R^{L}, R^{W}, R^{E} are H, R^{M} is OCF₃, R^{V} is CF₃, R^{X} is C₂H₅, and m is 2.
Table 318. Compounds of formula I-D-D8.3, wherein R^{L}, R^{V}, R^{W} are H, R^{E} is CH₃, R^{M} is OCF₃, R^{V} is CF₃, R^{X} is C₂H₅, and m is 2.
Table 319. Compounds of formula I-D-D8.3, wherein R^{L}, R^{W}, R^{E} are H, R^{M} is OCF₃, R^{V} is OCF₃, R^{X} is C₂H₅, and m is 2.
Table 320. Compounds of formula I-D-D8.3, wherein R^{L}, R^{V}, R^{W} are H, R^{E} is CH₃, R^{M} is OCF₃, R^{V} is OCF₃, R^{X} is C₂H₅, and m is 2.
Table 321. Compounds of formula I-D-D50.3, wherein R^{L}, R^{M}, R^{V}, R^{W}, R^{E} are H, R^{X} is C₂H₅, and m is 2.
Table 322. Compounds of formula I-D-D50.3, wherein R^{L}, R^{M}, R^{V}, R^{W} are H, R^{E} is CH₃, R^{X} is C₂H₅, and m is 2
Table 323. Compounds of formula I-D-D50.3, wherein R^{L}, R^{M}, R^{W}, R^{E} are H, R^{V} is CF₃, R^{X} is C₂H₅, and m is 2.
Table 324. Compounds of formula I-D-D50.3, wherein R^{L}, R^{M}, R^{V}, R^{W} are H, R^{E} is CH₃, R^{V} is CF₃, R^{X} is C₂H₅, and m is 2.
Table 325. Compounds of formula I-D-D50.3, wherein R^{L}, R^{M}, R^{W}, R^{E} are H, R^{V} is OCF₃, R^{X} is C₂H₅, and m is 2.
Table 326. Compounds of formula I-D-D50.3, wherein R^{L}, R^{M}, R^{V}, R^{W} are H, R^{E} is CH₃, R^{V} is OCF₃, R^{X} is C₂H₅, and m is 2.
Table 327. Compounds of formula I-D-D50.3, wherein R^{L}, R^{V}, R^{W}, R^{E} are H, R^{M} is CF₃, R^{X} is C₂H₅, and m is 2.
Table 328. Compounds of formula I-D-D50.3, wherein R^{L}, R^{V}, R^{W} are H, R^{M} is CF₃, R^{E} is CH₃, R^{X} is C₂H₅, and m is 2
Table 329. Compounds of formula I-D-D50.3, wherein R^{L}, R^{W}, R^{E} are H, R^{M} is CF₃, R^{V} is CF₃, R^{X} is C₂H₅, and m is 2.
Table 330. Compounds of formula I-D-D50.3, wherein R^{L}, R^{V}, R^{W} are H, R^{E} is CH₃, R^{M} is CF₃, R^{V} is CF₃, R^{X} is C₂H₅, and m is 2.
Table 331. Compounds of formula I-D-D50.3, wherein R^{L}, R^{W}, R^{E} are H, R^{M} is CF₃, R^{V} is OCF₃, R^{X} is C₂H₅, and m is 2.
Table 332. Compounds of formula I-D-D50.3, wherein R^{L}, R^{V}, R^{W} are H, R^{E} is CH₃, R^{M} is CF₃, R^{V} is OCF₃, R^{X} is C₂H₅, and m is 2.
Table 333. Compounds of formula I-D-D50.3, wherein R^{L}, R^{V}, R^{W}, R^{E} are H, R^{M} is OCF₃, R^{X} is C₂H₅, and m is 2.
Table 334. Compounds of formula I-D-D50.3, wherein R^{L}, R^{V}, R^{W} are H, R^{M} is OCF₃, R^{E} is CH₃, R^{X} is C₂H₅, and m is 2
Table 335. Compounds of formula I-D-D50.3, wherein R^{L}, R^{W}, R^{E} are H, R^{M} is OCF₃, R^{V} is OCF₃, R^{X} is C₂H₅, and m is 2.
Table 336. Compounds of formula I-D-D50.3, wherein R^{L}, R^{V}, R^{W} are H, R^{E} is CH₃, R^{M} is OCF₃, R^{V} is CF₃, R^{X} is C₂H₅, and m is 2.
Table 337. Compounds of formula I-D-D50.3, wherein R^{L}, R^{W}, R^{E} are H, R^{M} is OCF₃, R^{V} is OCF₃, R^{X} is C₂H₅, and m is 2.
Table 338. Compounds of formula I-D-D50.3, wherein R^{L}, R^{V}, R^{W} are H, R^{E} is CH₃, R^{M} is OCF₃, R^{V} is OCF₃, R^{X} is C₂H₅, and m is 2.

TABLE B
combinations of meanings for substituents R^{Q}, R^{T} and
R⁹; cPr = cyclopropyl; iPr = iso-propyl.

| Line | R^{Q} | R^{T} | R⁹ |
|---|---|---|---|
| 1 | H | H | CH₃ |
| 2 | H | H | CF₃ |
| 3 | H | H | OCH₃ |
| 4 | H | H | OCF₃ |
| 5 | H | H | F |
| 6 | H | H | Cl |
| 7 | H | H | Br |
| 8 | H | H | 1-CN-cPr |
| 9 | H | H | 1-CN-iPr |
| 10 | H | H | H |
| 11 | H | CF₃ | CH₃ |
| 12 | H | CF₃ | CF₃ |
| 13 | H | CF₃ | OCH₃ |
| 14 | H | CF₃ | OCF₃ |
| 15 | H | CF₃ | F |
| 16 | H | CF₃ | Br |
| 17 | H | CF₃ | 1-CN-cPr |
| 18 | H | CF₃ | 1-CN-iPr |
| 19 | H | CF₃ | Cl |
| 20 | H | CF₃ | H |
| 21 | H | OCF₃ | CH₃ |
| 22 | H | OCF₃ | CF₃ |
| 23 | H | OCF₃ | OCH₃ |
| 24 | H | OCF₃ | OCF₃ |
| 25 | H | OCF₃ | F |
| 26 | H | OCF₃ | Cl |
| 27 | H | OCF₃ | Br |
| 28 | H | OCF₃ | 1-CN-cPr |
| 29 | H | OCF₃ | 1-CN-iPr |
| 30 | H | OCF₃ | H |
| 31 | H | OCH₂CF₃ | CH₃ |
| 32 | H | OCH₂CF₃ | CF₃ |
| 33 | H | OCH₂CF₃ | OCH₃ |
| 34 | H | OCH₂CF₃ | OCF₃ |
| 35 | H | OCH₂CF₃ | F |
| 36 | H | OCH₂CF₃ | Cl |
| 37 | H | OCH₂CF₃ | Br |
| 38 | H | OCH₂CF₃ | 1-CN-cPr |
| 39 | H | OCH₂CF₃ | 1-CN-iPr |
| 40 | H | OCH₂CF₃ | H |
| 41 | H | OCH₂C₂F₅ | CH₃ |
| 42 | H | OCH₂C₂F₅ | CF₃ |
| 43 | H | OCH₂C₂F₅ | OCH₃ |
| 44 | H | OCH₂C₂F₅ | OCF₃ |
| 45 | H | OCH₂C₂F₅ | F |
| 46 | H | OCH₂C₂F₅ | Cl |
| 47 | H | OCH₂C₂F₅ | Br |
| 48 | H | OCH₂C₂F₅ | 1-CN-cPr |
| 49 | H | OCH₂C₂F₅ | 1-CN-iPr |
| 50 | H | OCH₂C₂F₅ | H |
| 51 | H | CH₃ | CH₃ |
| 52 | H | CH₃ | CF₃ |
| 53 | H | CH₃ | OCH₃ |
| 54 | H | CH₃ | OCF₃ |
| 55 | H | CH₃ | F |
| 56 | H | CH₃ | Cl |
| 57 | H | CH₃ | Br |
| 58 | H | CH₃ | 1-CN-cPr |
| 59 | H | CH₃ | 1-CN-iPr |
| 60 | H | CH₃ | H |
| 61 | H | OCH₃ | CH₃ |
| 62 | H | OCH₃ | CF₃ |
| 63 | H | OCH₃ | OCH₃ |
| 64 | H | OCH₃ | OCF₃ |
| 65 | H | OCH₃ | F |
| 66 | H | OCH₃ | Cl |
| 67 | H | OCH₃ | Br |
| 68 | H | OCH₃ | 1-CN-cPr |
| 69 | H | OCH₃ | 1-CN-iPr |
| 70 | H | OCH₃ | H |
| 71 | H | F | CH₃ |
| 72 | H | F | CF₃ |
| 73 | H | F | OCH₃ |
| 74 | H | F | OCF₃ |
| 75 | H | F | F |
| 76 | H | F | Cl |
| 77 | H | F | Br |
| 78 | H | F | 1-CN-cPr |
| 79 | H | F | 1-CN-iPr |
| 80 | H | F | H |
| 81 | H | Cl | CH₃ |
| 82 | H | Cl | CF₃ |
| 83 | H | Cl | OCH₃ |
| 84 | H | Cl | OCF₃ |
| 85 | H | Cl | F |
| 86 | H | Cl | Cl |
| 87 | H | Cl | Br |
| 88 | H | Cl | 1-CN-cPr |
| 89 | H | Cl | 1-CN-iPr |
| 90 | H | Cl | H |
| 91 | H | Br | CH₃ |
| 92 | H | Br | CF₃ |
| 93 | H | Br | OCH₃ |
| 94 | H | Br | OCF₃ |
| 95 | H | Br | F |
| 96 | H | Br | Cl |
| 97 | H | Br | Br |
| 98 | H | Br | 1-CN-cPr |
| 99 | H | Br | 1-CN-iPr |
| 100 | H | Br | H |
| 101 | H | SCF₃ | CH₃ |
| 102 | H | SCF₃ | CF₃ |
| 103 | H | SCF₃ | OCH₃ |
| 104 | H | SCF₃ | OCF₃ |
| 105 | H | SCF₃ | F |
| 106 | H | SCF₃ | Cl |
| 107 | H | SCF₃ | Br |
| 108 | H | SCF₃ | 1-CN-cPr |
| 109 | H | SCF₃ | 1-CN-iPr |
| 110 | H | SCF₃ | H |
| 111 | CF₃ | H | CH₃ |
| 112 | CF₃ | H | CF₃ |
| 113 | CF₃ | H | OCH₃ |
| 114 | CF₃ | H | OCF₃ |
| 115 | CF₃ | H | F |
| 116 | CF₃ | H | Cl |
| 117 | CF₃ | H | Br |
| 118 | CF₃ | H | 1-CN-cPr |
| 119 | CF₃ | H | 1-CN-iPr |
| 120 | CF₃ | H | H |
| 121 | CF₃ | CF₃ | CH₃ |
| 122 | CF₃ | CF₃ | CF₃ |
| 123 | CF₃ | CF₃ | OCH₃ |
| 124 | CF₃ | CF₃ | OCF₃ |
| 125 | CF₃ | CF₃ | F |
| 126 | CF₃ | CF₃ | Br |
| 127 | CF₃ | CF₃ | 1-CN-cPr |
| 128 | CF₃ | CF₃ | 1-CN-iPr |
| 129 | CF₃ | CF₃ | Cl |
| 130 | CF₃ | CF₃ | H |
| 131 | CF₃ | OCF₃ | CH₃ |
| 132 | CF₃ | OCF₃ | CF₃ |
| 133 | CF₃ | OCF₃ | OCH₃ |
| 134 | CF₃ | OCF₃ | OCF₃ |
| 135 | CF₃ | OCF₃ | F |
| 136 | CF₃ | OCF₃ | Cl |
| 137 | CF₃ | OCF₃ | Br |
| 138 | CF₃ | OCF₃ | 1-CN-cPr |
| 139 | CF₃ | OCF₃ | 1-CN-iPr |
| 140 | CF₃ | OCF₃ | H |
| 141 | CF₃ | OCH₂CF₃ | CH₃ |
| 142 | CF₃ | OCH₂CF₃ | CF₃ |
| 143 | CF₃ | OCH₂CF₃ | OCH₃ |
| 144 | CF₃ | OCH₂CF₃ | OCF₃ |
| 145 | CF₃ | OCH₂CF₃ | F |
| 146 | CF₃ | OCH₂CF₃ | Cl |
| 147 | CF₃ | OCH₂CF₃ | Br |
| 148 | CF₃ | OCH₂CF₃ | 1-CN-cPr |
| 149 | CF₃ | OCH₂CF₃ | 1-CN-iPr |
| 150 | CF₃ | OCH₂CF₃ | H |
| 151 | CF₃ | OCH₂C₂F₅ | CH₃ |
| 152 | CF₃ | OCH₂C₂F₅ | CF₃ |
| 153 | CF₃ | OCH₂C₂F₅ | OCH₃ |
| 154 | CF₃ | OCH₂C₂F₅ | OCF₃ |
| 155 | CF₃ | OCH₂C₂F₅ | F |
| 156 | CF₃ | OCH₂C₂F₅ | Cl |
| 157 | CF₃ | OCH₂C₂F₅ | Br |
| 158 | CF₃ | OCH₂C₂F₅ | 1-CN-cPr |
| 159 | CF₃ | OCH₂C₂F₅ | 1-CN-iPr |
| 160 | CF₃ | OCH₂C₂F₅ | H |
| 161 | CF₃ | CH₃ | CH₃ |
| 162 | CF₃ | CH₃ | CF₃ |
| 163 | CF₃ | CH₃ | OCH₃ |
| 164 | CF₃ | CH₃ | OCF₃ |
| 165 | CF₃ | CH₃ | F |
| 166 | CF₃ | CH₃ | Cl |
| 167 | CF₃ | CH₃ | Br |
| 168 | CF₃ | CH₃ | 1-CN-cPr |
| 169 | CF₃ | CH₃ | 1-CN-iPr |
| 170 | CF₃ | CH₃ | H |
| 171 | CF₃ | OCH₃ | CH₃ |
| 172 | CF₃ | OCH₃ | CF₃ |
| 173 | CF₃ | OCH₃ | OCH₃ |
| 174 | CF₃ | OCH₃ | OCF₃ |
| 175 | CF₃ | OCH₃ | F |
| 176 | CF₃ | OCH₃ | Cl |
| 177 | CF₃ | OCH₃ | Br |
| 178 | CF₃ | OCH₃ | 1-CN-cPr |
| 179 | CF₃ | OCH₃ | 1-CN-iPr |
| 180 | CF₃ | OCH₃ | H |
| 181 | CF₃ | F | CH₃ |
| 182 | CF₃ | F | CF₃ |
| 183 | CF₃ | F | OCH₃ |
| 184 | CF₃ | F | OCF₃ |
| 185 | CF₃ | F | F |
| 186 | CF₃ | F | Cl |
| 187 | CF₃ | F | Br |
| 188 | CF₃ | F | 1-CN-cPr |
| 189 | CF₃ | F | 1-CN-iPr |
| 190 | CF₃ | F | H |
| 191 | CF₃ | Cl | CH₃ |
| 192 | CF₃ | Cl | CF₃ |
| 193 | CF₃ | Cl | OCH₃ |
| 194 | CF₃ | Cl | OCF₃ |
| 195 | CF₃ | Cl | F |
| 196 | CF₃ | Cl | Cl |
| 197 | CF₃ | Cl | Br |
| 198 | CF₃ | Cl | 1-CN-cPr |
| 199 | CF₃ | Cl | 1-CN-iPr |
| 200 | CF₃ | Cl | H |
| 201 | CF₃ | Br | CH₃ |
| 202 | CF₃ | Br | CF₃ |
| 203 | CF₃ | Br | OCH₃ |
| 204 | CF₃ | Br | OCF₃ |
| 205 | CF₃ | Br | F |
| 206 | CF₃ | Br | Cl |
| 207 | CF₃ | Br | Br |
| 208 | CF₃ | Br | 1-CN-cPr |
| 209 | CF₃ | Br | 1-CN-iPr |
| 210 | CF₃ | Br | H |
| 211 | CF₃ | SCF₃ | CH₃ |
| 212 | CF₃ | SCF₃ | CF₃ |
| 213 | CF₃ | SCF₃ | OCH₃ |
| 214 | CF₃ | SCF₃ | OCF₃ |
| 215 | CF₃ | SCF₃ | F |
| 216 | CF₃ | SCF₃ | Cl |
| 217 | CF₃ | SCF₃ | Br |
| 218 | CF₃ | SCF₃ | 1-CN-cPr |
| 219 | CF₃ | SCF₃ | 1-CN-iPr |
| 220 | CF₃ | SCF₃ | H |
| 221 | OCF₃ | H | CH₃ |
| 222 | OCF₃ | H | CF₃ |
| 223 | OCF₃ | H | OCH₃ |
| 224 | OCF₃ | H | OCF₃ |
| 225 | OCF₃ | H | F |
| 226 | OCF₃ | H | Cl |
| 227 | OCF₃ | H | Br |
| 228 | OCF₃ | H | 1-CN-cPr |
| 229 | OCF₃ | H | 1-CN-iPr |
| 230 | OCF₃ | H | H |
| 231 | OCF₃ | CF₃ | CH₃ |
| 232 | OCF₃ | CF₃ | CF₃ |
| 233 | OCF₃ | CF₃ | OCH₃ |
| 234 | OCF₃ | CF₃ | OCF₃ |
| 235 | OCF₃ | CF₃ | F |
| 236 | OCF₃ | CF₃ | Br |
| 237 | OCF₃ | CF₃ | 1-CN-cPr |
| 238 | OCF₃ | CF₃ | 1-CN-iPr |
| 239 | OCF₃ | CF₃ | Cl |
| 240 | OCF₃ | CF₃ | H |
| 241 | OCF₃ | OCF₃ | CH₃ |
| 242 | OCF₃ | OCF₃ | CF₃ |
| 243 | OCF₃ | OCF₃ | OCH₃ |
| 244 | OCF₃ | OCF₃ | OCF₃ |
| 245 | OCF₃ | OCF₃ | F |
| 246 | OCF₃ | OCF₃ | Cl |
| 247 | OCF₃ | OCF₃ | Br |
| 248 | OCF₃ | OCF₃ | 1-CN-cPr |
| 249 | OCF₃ | OCF₃ | 1-CN-iPr |
| 250 | OCF₃ | OCF₃ | H |
| 251 | OCF₃ | OCH₂CF₃ | CH₃ |
| 252 | OCF₃ | OCH₂CF₃ | CF₃ |
| 253 | OCF₃ | OCH₂CF₃ | OCH₃ |
| 254 | OCF₃ | OCH₂CF₃ | OCF₃ |
| 255 | OCF₃ | OCH₂CF₃ | F |
| 256 | OCF₃ | OCH₂CF₃ | Cl |
| 257 | OCF₃ | OCH₂CF₃ | Br |
| 258 | OCF₃ | OCH₂CF₃ | 1-CN-cPr |
| 259 | OCF₃ | OCH₂CF₃ | 1-CN-iPr |
| 260 | OCF₃ | OCH₂CF₃ | H |
| 261 | OCF₃ | OCH₂C₂F₅ | CH₃ |
| 262 | OCF₃ | OCH₂C₂F₅ | CF₃ |
| 263 | OCF₃ | OCH₂C₂F₅ | OCH₃ |
| 264 | OCF₃ | OCH₂C₂F₅ | OCF₃ |
| 265 | OCF₃ | OCH₂C₂F₅ | F |
| 266 | OCF₃ | OCH₂C₂F₅ | Cl |
| 267 | OCF₃ | OCH₂C₂F₅ | Br |
| 268 | OCF₃ | OCH₂C₂F₅ | 1-CN-cPr |
| 269 | OCF₃ | OCH₂C₂F₅ | 1-CN-iPr |
| 270 | OCF₃ | OCH₂C₂F₅ | H |
| 271 | OCF₃ | CH₃ | CH₃ |
| 272 | OCF₃ | CH₃ | CF₃ |
| 273 | OCF₃ | CH₃ | OCH₃ |
| 274 | OCF₃ | CH₃ | OCF₃ |
| 275 | OCF₃ | CH₃ | F |
| 276 | OCF₃ | CH₃ | Cl |
| 277 | OCF₃ | CH₃ | Br |
| 278 | OCF₃ | CH₃ | 1-CN-cPr |
| 279 | OCF₃ | CH₃ | 1-CN-iPr |
| 280 | OCF₃ | CH₃ | H |
| 281 | OCF₃ | OCH₃ | CH₃ |
| 282 | OCF₃ | OCH₃ | CF₃ |
| 283 | OCF₃ | OCH₃ | OCH₃ |
| 284 | OCF₃ | OCH₃ | OCF₃ |
| 285 | OCF₃ | OCH₃ | F |
| 286 | OCF₃ | OCH₃ | Cl |
| 287 | OCF₃ | OCH₃ | Br |
| 288 | OCF₃ | OCH₃ | 1-CN-cPr |
| 289 | OCF₃ | OCH₃ | 1-CN-iPr |
| 290 | OCF₃ | OCH₃ | H |
| 291 | OCF₃ | F | CH₃ |
| 292 | OCF₃ | F | CF₃ |
| 293 | OCF₃ | F | OCH₃ |
| 294 | OCF₃ | F | OCF₃ |
| 295 | OCF₃ | F | F |
| 296 | OCF₃ | F | Cl |
| 297 | OCF₃ | F | Br |
| 298 | OCF₃ | F | 1-CN-cPr |
| 299 | OCF₃ | F | 1-CN-iPr |
| 300 | OCF₃ | F | H |
| 301 | OCF₃ | Cl | CH₃ |
| 302 | OCF₃ | Cl | CF₃ |
| 303 | OCF₃ | Cl | OCH₃ |
| 304 | OCF₃ | Cl | OCF₃ |
| 305 | OCF₃ | Cl | F |
| 306 | OCF₃ | Cl | Cl |
| 307 | OCF₃ | Cl | Br |
| 308 | OCF₃ | Cl | 1-CN-cPr |
| 309 | OCF₃ | Cl | 1-CN-iPr |
| 310 | OCF₃ | Cl | H |
| 311 | OCF₃ | Br | CH₃ |
| 312 | OCF₃ | Br | CF₃ |
| 313 | OCF₃ | Br | OCH₃ |
| 314 | OCF₃ | Br | OCF₃ |
| 315 | OCF₃ | Br | F |
| 316 | OCF₃ | Br | Cl |
| 317 | OCF₃ | Br | Br |
| 318 | OCF₃ | Br | 1-CN-cPr |
| 319 | OCF₃ | Br | 1-CN-iPr |
| 320 | OCF₃ | Br | H |
| 321 | OCF₃ | SCF₃ | CH₃ |
| 322 | OCF₃ | SCF₃ | CF₃ |
| 323 | OCF₃ | SCF₃ | OCH₃ |
| 324 | OCF₃ | SCF₃ | OCF₃ |
| 325 | OCF₃ | SCF₃ | F |
| 326 | OCF₃ | SCF₃ | Cl |
| 327 | OCF₃ | SCF₃ | Br |
| 328 | OCF₃ | SCF₃ | 1-CN-cPr |
| 329 | OCF₃ | SCF₃ | 1-CN-iPr |
| 330 | OCF₃ | SCF₃ | H |
| 331 | OCH₂CF₃ | H | CH₃ |
| 332 | OCH₂CF₃ | H | CF₃ |
| 333 | OCH₂CF₃ | H | OCH₃ |
| 334 | OCH₂CF₃ | H | OCF₃ |
| 335 | OCH₂CF₃ | H | F |
| 336 | OCH₂CF₃ | H | Cl |
| 337 | OCH₂CF₃ | H | Br |
| 338 | OCH₂CF₃ | H | 1-CN-cPr |
| 339 | OCH₂CF₃ | H | 1-CN-iPr |
| 340 | OCH₂CF₃ | H | H |
| 341 | OCH₂CF₃ | CF₃ | CH₃ |
| 342 | OCH₂CF₃ | CF₃ | CF₃ |
| 343 | OCH₂CF₃ | CF₃ | OCH₃ |
| 344 | OCH₂CF₃ | CF₃ | OCF₃ |
| 345 | OCH₂CF₃ | CF₃ | F |
| 346 | OCH₂CF₃ | CF₃ | Br |
| 347 | OCH₂CF₃ | CF₃ | 1-CN-cPr |
| 348 | OCH₂CF₃ | CF₃ | 1-CN-iPr |
| 349 | OCH₂CF₃ | CF₃ | Cl |
| 350 | OCH₂CF₃ | CF₃ | H |
| 351 | OCH₂CF₃ | OCF₃ | CH₃ |
| 352 | OCH₂CF₃ | OCF₃ | CF₃ |
| 353 | OCH₂CF₃ | OCF₃ | OCH₃ |
| 354 | OCH₂CF₃ | OCF₃ | OCF₃ |
| 355 | OCH₂CF₃ | OCF₃ | F |
| 356 | OCH₂CF₃ | OCF₃ | Cl |
| 357 | OCH₂CF₃ | OCF₃ | Br |
| 358 | OCH₂CF₃ | OCF₃ | 1-CN-cPr |
| 359 | OCH₂CF₃ | OCF₃ | 1-CN-iPr |
| 360 | OCH₂CF₃ | OCF₃ | H |
| 361 | OCH₂CF₃ | OCH₂CF₃ | CH₃ |
| 362 | OCH₂CF₃ | OCH₂CF₃ | CF₃ |
| 363 | OCH₂CF₃ | OCH₂CF₃ | OCH₃ |
| 364 | OCH₂CF₃ | OCH₂CF₃ | OCF₃ |
| 365 | OCH₂CF₃ | OCH₂CF₃ | F |
| 366 | OCH₂CF₃ | OCH₂CF₃ | Cl |
| 367 | OCH₂CF₃ | OCH₂CF₃ | Br |
| 368 | OCH₂CF₃ | OCH₂CF₃ | 1-CN-cPr |
| 369 | OCH₂CF₃ | OCH₂CF₃ | 1-CN-iPr |
| 370 | OCH₂CF₃ | OCH₂CF₃ | H |
| 371 | OCH₂CF₃ | OCH₂C₂F₅ | CH₃ |
| 372 | OCH₂CF₃ | OCH₂C₂F₅ | CF₃ |
| 373 | OCH₂CF₃ | OCH₂C₂F₅ | OCH₃ |
| 374 | OCH₂CF₃ | OCH₂C₂F₅ | OCF₃ |
| 375 | OCH₂CF₃ | OCH₂C₂F₅ | F |
| 376 | OCH₂CF₃ | OCH₂C₂F₅ | Cl |
| 377 | OCH₂CF₃ | OCH₂C₂F₅ | Br |
| 378 | OCH₂CF₃ | OCH₂C₂F₅ | 1-CN-cPr |
| 379 | OCH₂CF₃ | OCH₂C₂F₅ | 1-CN-iPr |
| 380 | OCH₂CF₃ | OCH₂C₂F₅ | H |
| 381 | OCH₂CF₃ | CH₃ | CH₃ |
| 382 | OCH₂CF₃ | CH₃ | CF₃ |
| 383 | OCH₂CF₃ | CH₃ | OCH₃ |
| 384 | OCH₂CF₃ | CH₃ | OCF₃ |
| 385 | OCH₂CF₃ | CH₃ | F |
| 386 | OCH₂CF₃ | CH₃ | Cl |
| 387 | OCH₂CF₃ | CH₃ | Br |
| 388 | OCH₂CF₃ | CH₃ | 1-CN-cPr |
| 389 | OCH₂CF₃ | CH₃ | 1-CN-iPr |
| 390 | OCH₂CF₃ | CH₃ | H |
| 391 | OCH₂CF₃ | OCH₃ | CH₃ |
| 392 | OCH₂CF₃ | OCH₃ | CF₃ |
| 393 | OCH₂CF₃ | OCH₃ | OCH₃ |
| 394 | OCH₂CF₃ | OCH₃ | OCF₃ |
| 395 | OCH₂CF₃ | OCH₃ | F |
| 396 | OCH₂CF₃ | OCH₃ | Cl |
| 397 | OCH₂CF₃ | OCH₃ | Br |
| 398 | OCH₂CF₃ | OCH₃ | 1-CN-cPr |
| 399 | OCH₂CF₃ | OCH₃ | 1-CN-iPr |
| 400 | OCH₂CF₃ | OCH₃ | H |
| 401 | OCH₂CF₃ | F | CH₃ |
| 402 | OCH₂CF₃ | F | CF₃ |
| 403 | OCH₂CF₃ | F | OCH₃ |
| 404 | OCH₂CF₃ | F | OCF₃ |
| 405 | OCH₂CF₃ | F | F |
| 406 | OCH₂CF₃ | F | Cl |
| 407 | OCH₂CF₃ | F | Br |
| 408 | OCH₂CF₃ | F | 1-CN-cPr |
| 409 | OCH₂CF₃ | F | 1-CN-iPr |
| 410 | OCH₂CF₃ | F | H |
| 411 | OCH₂CF₃ | Cl | CH₃ |
| 412 | OCH₂CF₃ | Cl | CF₃ |
| 413 | OCH₂CF₃ | Cl | OCH₃ |
| 414 | OCH₂CF₃ | Cl | OCF₃ |
| 415 | OCH₂CF₃ | Cl | F |
| 416 | OCH₂CF₃ | Cl | Cl |
| 417 | OCH₂CF₃ | Cl | Br |
| 418 | OCH₂CF₃ | Cl | 1-CN-cPr |
| 419 | OCH₂CF₃ | Cl | 1-CN-iPr |
| 420 | OCH₂CF₃ | Cl | H |
| 421 | OCH₂CF₃ | Br | CH₃ |
| 422 | OCH₂CF₃ | Br | CF₃ |
| 423 | OCH₂CF₃ | Br | OCH₃ |
| 424 | OCH₂CF₃ | Br | OCF₃ |
| 425 | OCH₂CF₃ | Br | F |
| 426 | OCH₂CF₃ | Br | Cl |
| 427 | OCH₂CF₃ | Br | Br |
| 428 | OCH₂CF₃ | Br | 1-CN-cPr |
| 429 | OCH₂CF₃ | Br | 1-CN-iPr |
| 430 | OCH₂CF₃ | Br | H |
| 431 | OCH₂CF₃ | SCF₃ | CH₃ |
| 432 | OCH₂CF₃ | SCF₃ | CF₃ |
| 433 | OCH₂CF₃ | SCF₃ | OCH₃ |
| 434 | OCH₂CF₃ | SCF₃ | OCF₃ |
| 435 | OCH₂CF₃ | SCF₃ | F |
| 436 | OCH₂CF₃ | SCF₃ | Cl |
| 437 | OCH₂CF₃ | SCF₃ | Br |
| 438 | OCH₂CF₃ | SCF₃ | 1-CN-cPr |
| 439 | OCH₂CF₃ | SCF₃ | 1-CN-iPr |
| 440 | OCH₂CF₃ | SCF₃ | H |
| 441 | OCH₂C₂F₅ | H | CH₃ |
| 442 | OCH₂C₂F₅ | H | CF₃ |
| 443 | OCH₂C₂F₅ | H | OCH₃ |
| 444 | OCH₂C₂F₅ | H | OCF₃ |
| 445 | OCH₂C₂F₅ | H | F |
| 446 | OCH₂C₂F₅ | H | Cl |
| 447 | OCH₂C₂F₅ | H | Br |
| 448 | OCH₂C₂F₅ | H | 1-CN-cPr |
| 449 | OCH₂C₂F₅ | H | 1-CN-iPr |
| 450 | OCH₂C₂F₅ | H | H |
| 451 | OCH₂C₂F₅ | CF₃ | CH₃ |
| 452 | OCH₂C₂F₅ | CF₃ | CF₃ |
| 453 | OCH₂C₂F₅ | CF₃ | OCH₃ |
| 454 | OCH₂C₂F₅ | CF₃ | OCF₃ |
| 455 | OCH₂C₂F₅ | CF₃ | F |
| 456 | OCH₂C₂F₅ | CF₃ | Br |
| 457 | OCH₂C₂F₅ | CF₃ | 1-CN-cPr |
| 458 | OCH₂C₂F₅ | CF₃ | 1-CN-iPr |
| 459 | OCH₂C₂F₅ | CF₃ | Cl |
| 460 | OCH₂C₂F₅ | CF₃ | H |
| 461 | OCH₂C₂F₅ | OCF₃ | CH₃ |
| 462 | OCH₂C₂F₅ | OCF₃ | CF₃ |
| 463 | OCH₂C₂F₅ | OCF₃ | OCH₃ |
| 464 | OCH₂C₂F₅ | OCF₃ | OCF₃ |
| 465 | OCH₂C₂F₅ | OCF₃ | F |
| 466 | OCH₂C₂F₅ | OCF₃ | Cl |
| 467 | OCH₂C₂F₅ | OCF₃ | Br |
| 468 | OCH₂C₂F₅ | OCF₃ | 1-CN-cPr |
| 469 | OCH₂C₂F₅ | OCF₃ | 1-CN-iPr |
| 470 | OCH₂C₂F₅ | OCF₃ | H |
| 471 | OCH₂C₂F₅ | OCH₂CF₃ | CH₃ |
| 472 | OCH₂C₂F₅ | OCH₂CF₃ | CF₃ |
| 473 | OCH₂C₂F₅ | OCH₂CF₃ | OCH₃ |
| 474 | OCH₂C₂F₅ | OCH₂CF₃ | OCF₃ |
| 475 | OCH₂C₂F₅ | OCH₂CF₃ | F |
| 476 | OCH₂C₂F₅ | OCH₂CF₃ | Cl |
| 477 | OCH₂C₂F₅ | OCH₂CF₃ | Br |
| 478 | OCH₂C₂F₅ | OCH₂CF₃ | 1-CN-cPr |
| 479 | OCH₂C₂F₅ | OCH₂CF₃ | 1-CN-iPr |
| 480 | OCH₂C₂F₅ | OCH₂CF₃ | H |
| 481 | OCH₂C₂F₅ | OCH₂C₂F₅ | CH₃ |
| 482 | OCH₂C₂F₅ | OCH₂C₂F₅ | CF₃ |
| 483 | OCH₂C₂F₅ | OCH₂C₂F₅ | OCH₃ |
| 484 | OCH₂C₂F₅ | OCH₂C₂F₅ | OCF₃ |
| 485 | OCH₂C₂F₅ | OCH₂C₂F₅ | F |
| 486 | OCH₂C₂F₅ | OCH₂C₂F₅ | Cl |
| 487 | OCH₂C₂F₅ | OCH₂C₂F₅ | Br |
| 488 | OCH₂C₂F₅ | OCH₂C₂F₅ | 1-CN-cPr |
| 489 | OCH₂C₂F₅ | OCH₂C₂F₅ | 1-CN-iPr |
| 490 | OCH₂C₂F₅ | OCH₂C₂F₅ | H |
| 491 | OCH₂C₂F₅ | CH₃ | CH₃ |
| 492 | OCH₂C₂F₅ | CH₃ | CF₃ |
| 493 | OCH₂C₂F₅ | CH₃ | OCH₃ |
| 494 | OCH₂C₂F₅ | CH₃ | OCF₃ |
| 495 | OCH₂C₂F₅ | CH₃ | F |
| 496 | OCH₂C₂F₅ | CH₃ | Cl |
| 497 | OCH₂C₂F₅ | CH₃ | Br |
| 498 | OCH₂C₂F₅ | CH₃ | 1-CN-cPr |
| 499 | OCH₂C₂F₅ | CH₃ | 1-CN-iPr |
| 500 | OCH₂C₂F₅ | CH₃ | H |
| 501 | OCH₂C₂F₅ | OCH₃ | CH₃ |
| 502 | OCH₂C₂F₅ | OCH₃ | CF₃ |
| 503 | OCH₂C₂F₅ | OCH₃ | OCH₃ |
| 504 | OCH₂C₂F₅ | OCH₃ | OCF₃ |
| 505 | OCH₂C₂F₅ | OCH₃ | F |
| 506 | OCH₂C₂F₅ | OCH₃ | Cl |
| 507 | OCH₂C₂F₅ | OCH₃ | Br |
| 508 | OCH₂C₂F₅ | OCH₃ | 1-CN-cPr |
| 509 | OCH₂C₂F₅ | OCH₃ | 1-CN-iPr |
| 510 | OCH₂C₂F₅ | OCH₃ | H |
| 511 | OCH₂C₂F₅ | F | CH₃ |
| 512 | OCH₂C₂F₅ | F | CF₃ |
| 513 | OCH₂C₂F₅ | F | OCH₃ |
| 514 | OCH₂C₂F₅ | F | OCF₃ |
| 515 | OCH₂C₂F₅ | F | F |
| 516 | OCH₂C₂F₅ | F | Cl |
| 517 | OCH₂C₂F₅ | F | Br |
| 518 | OCH₂C₂F₅ | F | 1-CN-cPr |
| 519 | OCH₂C₂F₅ | F | 1-CN-iPr |
| 520 | OCH₂C₂F₅ | F | H |
| 521 | OCH₂C₂F₅ | Cl | CH₃ |
| 522 | OCH₂C₂F₅ | Cl | CF₃ |
| 523 | OCH₂C₂F₅ | Cl | OCH₃ |
| 524 | OCH₂C₂F₅ | Cl | OCF₃ |
| 525 | OCH₂C₂F₅ | Cl | F |
| 526 | OCH₂C₂F₅ | Cl | Cl |
| 527 | OCH₂C₂F₅ | Cl | Br |
| 528 | OCH₂C₂F₅ | Cl | 1-CN-cPr |
| 529 | OCH₂C₂F₅ | Cl | 1-CN-iPr |
| 530 | OCH₂C₂F₅ | Cl | H |
| 531 | OCH₂C₂F₅ | Br | CH₃ |
| 532 | OCH₂C₂F₅ | Br | CF₃ |
| 533 | OCH₂C₂F₅ | Br | OCH₃ |
| 534 | OCH₂C₂F₅ | Br | OCF₃ |
| 535 | OCH₂C₂F₅ | Br | F |
| 536 | OCH₂C₂F₅ | Br | Cl |
| 537 | OCH₂C₂F₅ | Br | Br |
| 538 | OCH₂C₂F₅ | Br | 1-CN-cPr |
| 539 | OCH₂C₂F₅ | Br | 1-CN-iPr |
| 540 | OCH₂C₂F₅ | Br | H |
| 541 | OCH₂C₂F₅ | SCF₃ | CH₃ |
| 542 | OCH₂C₂F₅ | SCF₃ | CF₃ |
| 543 | OCH₂C₂F₅ | SCF₃ | OCH₃ |
| 544 | OCH₂C₂F₅ | SCF₃ | OCF₃ |
| 545 | OCH₂C₂F₅ | SCF₃ | F |
| 546 | OCH₂C₂F₅ | SCF₃ | Cl |
| 547 | OCH₂C₂F₅ | SCF₃ | Br |
| 548 | OCH₂C₂F₅ | SCF₃ | 1-CN-cPr |
| 549 | OCH₂C₂F₅ | SCF₃ | 1-CN-iPr |
| 550 | OCH₂C₂F₅ | SCF₃ | H |
| 551 | CH₃ | H | CH₃ |
| 552 | CH₃ | H | CF₃ |
| 553 | CH₃ | H | OCH₃ |
| 554 | CH₃ | H | OCF₃ |
| 555 | CH₃ | H | F |
| 556 | CH₃ | H | Cl |
| 557 | CH₃ | H | Br |
| 558 | CH₃ | H | 1-CN-cPr |
| 559 | CH₃ | H | 1-CN-iPr |
| 560 | CH₃ | H | H |
| 561 | CH₃ | CF₃ | CH₃ |
| 562 | CH₃ | CF₃ | CF₃ |
| 563 | CH₃ | CF₃ | OCH₃ |
| 564 | CH₃ | CF₃ | OCF₃ |
| 565 | CH₃ | CF₃ | F |
| 566 | CH₃ | CF₃ | Br |
| 567 | CH₃ | CF₃ | 1-CN-cPr |
| 568 | CH₃ | CF₃ | 1-CN-iPr |
| 569 | CH₃ | CF₃ | Cl |
| 570 | CH₃ | CF₃ | H |
| 571 | CH₃ | OCF₃ | CH₃ |
| 572 | CH₃ | OCF₃ | CF₃ |
| 573 | CH₃ | OCF₃ | OCH₃ |
| 574 | CH₃ | OCF₃ | OCF₃ |
| 575 | CH₃ | OCF₃ | F |
| 576 | CH₃ | OCF₃ | Cl |
| 577 | CH₃ | OCF₃ | Br |
| 578 | CH₃ | OCF₃ | 1-CN-cPr |
| 579 | CH₃ | OCF₃ | 1-CN-iPr |
| 580 | CH₃ | OCF₃ | H |
| 581 | CH₃ | OCH₂CF₃ | CH₃ |
| 582 | CH₃ | OCH₂CF₃ | CF₃ |
| 583 | CH₃ | OCH₂CF₃ | OCH₃ |
| 584 | CH₃ | OCH₂CF₃ | OCF₃ |
| 585 | CH₃ | OCH₂CF₃ | F |
| 586 | CH₃ | OCH₂CF₃ | Cl |
| 587 | CH₃ | OCH₂CF₃ | Br |
| 588 | CH₃ | OCH₂CF₃ | 1-CN-cPr |
| 589 | CH₃ | OCH₂CF₃ | 1-CN-iPr |
| 590 | CH₃ | OCH₂CF₃ | H |
| 591 | CH₃ | OCH₂C₂F₅ | CH₃ |
| 592 | CH₃ | OCH₂C₂F₅ | CF₃ |
| 593 | CH₃ | OCH₂C₂F₅ | OCH₃ |
| 594 | CH₃ | OCH₂C₂F₅ | OCF₃ |
| 595 | CH₃ | OCH₂C₂F₅ | F |
| 596 | CH₃ | OCH₂C₂F₅ | Cl |
| 597 | CH₃ | OCH₂C₂F₅ | Br |
| 598 | CH₃ | OCH₂C₂F₅ | 1-CN-cPr |
| 599 | CH₃ | OCH₂C₂F₅ | 1-CN-iPr |
| 600 | CH₃ | OCH₂C₂F₅ | H |
| 601 | CH₃ | CH₃ | CH₃ |
| 602 | CH₃ | CH₃ | CF₃ |
| 603 | CH₃ | CH₃ | OCH₃ |
| 604 | CH₃ | CH₃ | OCF₃ |
| 605 | CH₃ | CH₃ | F |
| 606 | CH₃ | CH₃ | Cl |
| 607 | CH₃ | CH₃ | Br |
| 608 | CH₃ | CH₃ | 1-CN-cPr |
| 609 | CH₃ | CH₃ | 1-CN-iPr |
| 610 | CH₃ | CH₃ | H |
| 611 | CH₃ | OCH₃ | CH₃ |
| 612 | CH₃ | OCH₃ | CF₃ |
| 613 | CH₃ | OCH₃ | OCH₃ |
| 614 | CH₃ | OCH₃ | OCF₃ |
| 615 | CH₃ | OCH₃ | F |
| 616 | CH₃ | OCH₃ | Cl |
| 617 | CH₃ | OCH₃ | Br |
| 618 | CH₃ | OCH₃ | 1-CN-cPr |
| 619 | CH₃ | OCH₃ | 1-CN-iPr |
| 620 | CH₃ | OCH₃ | H |
| 621 | CH₃ | F | CH₃ |
| 622 | CH₃ | F | CF₃ |
| 623 | CH₃ | F | OCH₃ |
| 624 | CH₃ | F | OCF₃ |
| 625 | CH₃ | F | F |
| 626 | CH₃ | F | Cl |
| 627 | CH₃ | F | Br |
| 628 | CH₃ | F | 1-CN-cPr |
| 629 | CH₃ | F | 1-CN-iPr |
| 630 | CH₃ | F | H |
| 631 | CH₃ | Cl | CH₃ |
| 632 | CH₃ | Cl | CF₃ |
| 633 | CH₃ | Cl | OCH₃ |
| 634 | CH₃ | Cl | OCF₃ |
| 635 | CH₃ | Cl | F |
| 636 | CH₃ | Cl | Cl |
| 637 | CH₃ | Cl | Br |
| 638 | CH₃ | Cl | 1-CN-cPr |
| 639 | CH₃ | Cl | 1-CN-iPr |
| 640 | CH₃ | Cl | H |
| 641 | CH₃ | Br | CH₃ |
| 642 | CH₃ | Br | CF₃ |
| 643 | CH₃ | Br | OCH₃ |
| 644 | CH₃ | Br | OCF₃ |
| 645 | CH₃ | Br | F |
| 646 | CH₃ | Br | Cl |
| 647 | CH₃ | Br | Br |
| 648 | CH₃ | Br | 1-CN-cPr |
| 649 | CH₃ | Br | 1-CN-iPr |
| 650 | CH₃ | Br | H |
| 651 | CH₃ | SCF₃ | CH₃ |
| 652 | CH₃ | SCF₃ | CF₃ |
| 653 | CH₃ | SCF₃ | OCH₃ |
| 654 | CH₃ | SCF₃ | OCF₃ |
| 655 | CH₃ | SCF₃ | F |
| 656 | CH₃ | SCF₃ | Cl |
| 657 | CH₃ | SCF₃ | Br |
| 658 | CH₃ | SCF₃ | 1-CN-cPr |
| 659 | CH₃ | SCF₃ | 1-CN-iPr |
| 660 | CH₃ | SCF₃ | H |
| 661 | OCH₃ | H | CH₃ |
| 662 | OCH₃ | H | CF₃ |
| 663 | OCH₃ | H | OCH₃ |
| 664 | OCH₃ | H | OCF₃ |
| 665 | OCH₃ | H | F |
| 666 | OCH₃ | H | Cl |
| 667 | OCH₃ | H | Br |
| 668 | OCH₃ | H | 1-CN-cPr |
| 669 | OCH₃ | H | 1-CN-iPr |
| 670 | OCH₃ | H | H |
| 671 | OCH₃ | CF₃ | CH₃ |
| 672 | OCH₃ | CF₃ | CF₃ |
| 673 | OCH₃ | CF₃ | OCH₃ |
| 674 | OCH₃ | CF₃ | OCF₃ |
| 675 | OCH₃ | CF₃ | F |
| 676 | OCH₃ | CF₃ | Br |
| 677 | OCH₃ | CF₃ | 1-CN-cPr |
| 678 | OCH₃ | CF₃ | 1-CN-iPr |
| 679 | OCH₃ | CF₃ | Cl |
| 680 | OCH₃ | CF₃ | H |
| 681 | OCH₃ | OCF₃ | CH₃ |
| 682 | OCH₃ | OCF₃ | CF₃ |
| 683 | OCH₃ | OCF₃ | OCH₃ |
| 684 | OCH₃ | OCF₃ | OCF₃ |
| 685 | OCH₃ | OCF₃ | F |
| 686 | OCH₃ | OCF₃ | Cl |
| 687 | OCH₃ | OCF₃ | Br |
| 688 | OCH₃ | OCF₃ | 1-CN-cPr |
| 689 | OCH₃ | OCF₃ | 1-CN-iPr |
| 690 | OCH₃ | OCF₃ | H |
| 691 | OCH₃ | OCH₂CF₃ | CH₃ |
| 692 | OCH₃ | OCH₂CF₃ | CF₃ |
| 693 | OCH₃ | OCH₂CF₃ | OCH₃ |
| 694 | OCH₃ | OCH₂CF₃ | OCF₃ |
| 695 | OCH₃ | OCH₂CF₃ | F |
| 696 | OCH₃ | OCH₂CF₃ | Cl |
| 697 | OCH₃ | OCH₂CF₃ | Br |
| 698 | OCH₃ | OCH₂CF₃ | 1-CN-cPr |
| 699 | OCH₃ | OCH₂CF₃ | 1-CN-iPr |
| 700 | OCH₃ | OCH₂CF₃ | H |
| 701 | OCH₃ | OCH₂C₂F₅ | CH₃ |
| 702 | OCH₃ | OCH₂C₂F₅ | CF₃ |
| 703 | OCH₃ | OCH₂C₂F₅ | OCH₃ |
| 704 | OCH₃ | OCH₂C₂F₅ | OCF₃ |
| 705 | OCH₃ | OCH₂C₂F₅ | F |
| 706 | OCH₃ | OCH₂C₂F₅ | Cl |
| 707 | OCH₃ | OCH₂C₂F₅ | Br |
| 708 | OCH₃ | OCH₂C₂F₅ | 1-CN-cPr |
| 709 | OCH₃ | OCH₂C₂F₅ | 1-CN-iPr |
| 710 | OCH₃ | OCH₂C₂F₅ | H |
| 711 | OCH₃ | CH₃ | CH₃ |
| 712 | OCH₃ | CH₃ | CF₃ |
| 713 | OCH₃ | CH₃ | OCH₃ |
| 714 | OCH₃ | CH₃ | OCF₃ |
| 715 | OCH₃ | CH₃ | F |
| 716 | OCH₃ | CH₃ | Cl |
| 717 | OCH₃ | CH₃ | Br |
| 718 | OCH₃ | CH₃ | 1-CN-cPr |
| 719 | OCH₃ | CH₃ | 1-CN-iPr |
| 720 | OCH₃ | CH₃ | H |
| 721 | OCH₃ | OCH₃ | CH₃ |
| 722 | OCH₃ | OCH₃ | CF₃ |
| 723 | OCH₃ | OCH₃ | OCH₃ |
| 724 | OCH₃ | OCH₃ | OCF₃ |
| 725 | OCH₃ | OCH₃ | F |
| 726 | OCH₃ | OCH₃ | Cl |
| 727 | OCH₃ | OCH₃ | Br |
| 728 | OCH₃ | OCH₃ | 1-CN-cPr |
| 729 | OCH₃ | OCH₃ | 1-CN-iPr |
| 730 | OCH₃ | OCH₃ | H |
| 731 | OCH₃ | F | CH₃ |
| 732 | OCH₃ | F | CF₃ |
| 733 | OCH₃ | F | OCH₃ |
| 734 | OCH₃ | F | OCF₃ |
| 735 | OCH₃ | F | F |
| 736 | OCH₃ | F | Cl |
| 737 | OCH₃ | F | Br |
| 738 | OCH₃ | F | 1-CN-cPr |
| 739 | OCH₃ | F | 1-CN-iPr |
| 740 | OCH₃ | F | H |
| 741 | OCH₃ | Cl | CH₃ |
| 742 | OCH₃ | Cl | CF₃ |
| 743 | OCH₃ | Cl | OCH₃ |
| 744 | OCH₃ | Cl | OCF₃ |
| 745 | OCH₃ | Cl | F |
| 746 | OCH₃ | Cl | Cl |
| 747 | OCH₃ | Cl | Br |
| 748 | OCH₃ | Cl | 1-CN-cPr |
| 749 | OCH₃ | Cl | 1-CN-iPr |
| 750 | OCH₃ | Cl | H |
| 751 | OCH₃ | Br | CH₃ |
| 752 | OCH₃ | Br | CF₃ |
| 753 | OCH₃ | Br | OCH₃ |
| 754 | OCH₃ | Br | OCF₃ |
| 755 | OCH₃ | Br | F |
| 756 | OCH₃ | Br | Cl |
| 757 | OCH₃ | Br | Br |
| 758 | OCH₃ | Br | 1-CN-cPr |
| 759 | OCH₃ | Br | 1-CN-iPr |
| 760 | OCH₃ | Br | H |
| 761 | OCH₃ | SCF₃ | CH₃ |
| 762 | OCH₃ | SCF₃ | CF₃ |
| 763 | OCH₃ | SCF₃ | OCH₃ |
| 764 | OCH₃ | SCF₃ | OCF₃ |
| 765 | OCH₃ | SCF₃ | F |
| 766 | OCH₃ | SCF₃ | Cl |
| 767 | OCH₃ | SCF₃ | Br |
| 768 | OCH₃ | SCF₃ | 1-CN-cPr |
| 769 | OCH₃ | SCF₃ | 1-CN-iPr |
| 770 | OCH₃ | SCF₃ | H |
| 771 | F | H | CH₃ |
| 772 | F | H | CF₃ |
| 773 | F | H | OCH₃ |
| 774 | F | H | OCF₃ |
| 775 | F | H | F |
| 776 | F | H | Cl |
| 777 | F | H | Br |
| 778 | F | H | 1-CN-cPr |
| 779 | F | H | 1-CN-iPr |
| 780 | F | H | H |
| 781 | F | CF₃ | CH₃ |
| 782 | F | CF₃ | CF₃ |
| 783 | F | CF₃ | OCH₃ |
| 784 | F | CF₃ | OCF₃ |
| 785 | F | CF₃ | F |
| 786 | F | CF₃ | Br |
| 787 | F | CF₃ | 1-CN-cPr |
| 788 | F | CF₃ | 1-CN-iPr |
| 789 | F | CF₃ | Cl |
| 790 | F | CF₃ | H |
| 791 | F | OCF₃ | CH₃ |
| 792 | F | OCF₃ | CF₃ |
| 793 | F | OCF₃ | OCH₃ |
| 794 | F | OCF₃ | OCF₃ |
| 795 | F | OCF₃ | F |
| 796 | F | OCF₃ | Cl |
| 797 | F | OCF₃ | Br |
| 798 | F | OCF₃ | 1-CN-cPr |
| 799 | F | OCF₃ | 1-CN-iPr |
| 800 | F | OCF₃ | H |
| 801 | F | OCH₂CF₃ | CH₃ |
| 802 | F | OCH₂CF₃ | CF₃ |
| 803 | F | OCH₂CF₃ | OCH₃ |
| 804 | F | OCH₂CF₃ | OCF₃ |
| 805 | F | OCH₂CF₃ | F |
| 806 | F | OCH₂CF₃ | Cl |
| 807 | F | OCH₂CF₃ | Br |
| 808 | F | OCH₂CF₃ | 1-CN-cPr |
| 809 | F | OCH₂CF₃ | 1-CN-iPr |
| 810 | F | OCH₂CF₃ | H |
| 811 | F | OCH₂C₂F₅ | CH₃ |
| 812 | F | OCH₂C₂F₅ | CF₃ |
| 813 | F | OCH₂C₂F₅ | OCH₃ |
| 814 | F | OCH₂C₂F₅ | OCF₃ |
| 815 | F | OCH₂C₂F₅ | F |
| 816 | F | OCH₂C₂F₅ | Cl |
| 817 | F | OCH₂C₂F₅ | Br |
| 818 | F | OCH₂C₂F₅ | 1-CN-cPr |
| 819 | F | OCH₂C₂F₅ | 1-CN-iPr |
| 820 | F | OCH₂C₂F₅ | H |
| 821 | F | CH₃ | CH₃ |
| 822 | F | CH₃ | CF₃ |
| 823 | F | CH₃ | OCH₃ |
| 824 | F | CH₃ | OCF₃ |
| 825 | F | CH₃ | F |
| 826 | F | CH₃ | Cl |
| 827 | F | CH₃ | Br |
| 828 | F | CH₃ | 1-CN-cPr |
| 829 | F | CH₃ | 1-CN-iPr |
| 830 | F | CH₃ | H |
| 831 | F | OCH₃ | CH₃ |
| 832 | F | OCH₃ | CF₃ |
| 833 | F | OCH₃ | OCH₃ |
| 834 | F | OCH₃ | OCF₃ |
| 835 | F | OCH₃ | F |
| 836 | F | OCH₃ | Cl |
| 837 | F | OCH₃ | Br |
| 838 | F | OCH₃ | 1-CN-cPr |
| 839 | F | OCH₃ | 1-CN-iPr |
| 840 | F | OCH₃ | H |
| 841 | F | F | CH₃ |
| 842 | F | F | CF₃ |
| 843 | F | F | OCH₃ |
| 844 | F | F | OCF₃ |
| 845 | F | F | F |
| 846 | F | F | Cl |
| 847 | F | F | Br |
| 848 | F | F | 1-CN-cPr |
| 849 | F | F | 1-CN-iPr |
| 850 | F | F | H |
| 851 | F | Cl | CH₃ |
| 852 | F | Cl | CF₃ |
| 853 | F | Cl | OCH₃ |
| 854 | F | Cl | OCF₃ |
| 855 | F | Cl | F |
| 856 | F | Cl | Cl |
| 857 | F | Cl | Br |
| 858 | F | Cl | 1-CN-cPr |
| 859 | F | Cl | 1-CN-iPr |
| 860 | F | Cl | H |
| 861 | F | Br | CH₃ |
| 862 | F | Br | CF₃ |
| 863 | F | Br | OCH₃ |
| 864 | F | Br | OCF₃ |
| 865 | F | Br | F |
| 866 | F | Br | Cl |
| 867 | F | Br | Br |
| 868 | F | Br | 1-CN-cPr |
| 869 | F | Br | 1-CN-iPr |
| 870 | F | Br | H |
| 871 | F | SCF₃ | CH₃ |
| 872 | F | SCF₃ | CF₃ |
| 873 | F | SCF₃ | OCH₃ |
| 874 | F | SCF₃ | OCF₃ |
| 875 | F | SCF₃ | F |
| 876 | F | SCF₃ | Cl |
| 877 | F | SCF₃ | Br |
| 878 | F | SCF₃ | 1-CN-cPr |
| 879 | F | SCF₃ | 1-CN-iPr |
| 880 | F | SCF₃ | H |
| 881 | Cl | H | CH₃ |
| 882 | Cl | H | CF₃ |
| 883 | Cl | H | OCH₃ |
| 884 | Cl | H | OCF₃ |
| 885 | Cl | H | F |
| 886 | Cl | H | Cl |
| 887 | Cl | H | Br |
| 888 | Cl | H | 1-CN-cPr |
| 889 | Cl | H | 1-CN-iPr |
| 890 | Cl | H | H |
| 891 | Cl | CF₃ | CH₃ |
| 892 | Cl | CF₃ | CF₃ |
| 893 | Cl | CF₃ | OCH₃ |
| 894 | Cl | CF₃ | OCF₃ |
| 895 | Cl | CF₃ | F |
| 896 | Cl | CF₃ | Br |
| 897 | Cl | CF₃ | 1-CN-cPr |
| 898 | Cl | CF₃ | 1-CN-iPr |
| 899 | Cl | CF₃ | Cl |
| 900 | Cl | CF₃ | H |
| 901 | Cl | OCF₃ | CH₃ |
| 902 | Cl | OCF₃ | CF₃ |
| 903 | Cl | OCF₃ | OCH₃ |
| 904 | Cl | OCF₃ | OCF₃ |
| 905 | Cl | OCF₃ | F |
| 906 | Cl | OCF₃ | Cl |
| 907 | Cl | OCF₃ | Br |
| 908 | Cl | OCF₃ | 1-CN-cPr |
| 909 | Cl | OCF₃ | 1-CN-iPr |
| 910 | Cl | OCF₃ | H |
| 911 | Cl | OCH₂CF₃ | CH₃ |
| 912 | Cl | OCH₂CF₃ | CF₃ |
| 913 | Cl | OCH₂CF₃ | OCH₃ |
| 914 | Cl | OCH₂CF₃ | OCF₃ |
| 915 | Cl | OCH₂CF₃ | F |
| 916 | Cl | OCH₂CF₃ | Cl |
| 917 | Cl | OCH₂CF₃ | Br |
| 918 | Cl | OCH₂CF₃ | 1-CN-cPr |
| 919 | Cl | OCH₂CF₃ | 1-CN-iPr |
| 920 | Cl | OCH₂CF₃ | H |
| 921 | Cl | OCH₂C₂F₅ | CH₃ |
| 922 | Cl | OCH₂C₂F₅ | CF₃ |
| 923 | Cl | OCH₂C₂F₅ | OCH₃ |
| 924 | Cl | OCH₂C₂F₅ | OCF₃ |
| 925 | Cl | OCH₂C₂F₅ | F |
| 926 | Cl | OCH₂C₂F₅ | Cl |
| 927 | Cl | OCH₂C₂F₅ | Br |
| 928 | Cl | OCH₂C₂F₅ | 1-CN-cPr |
| 929 | Cl | OCH₂C₂F₅ | 1-CN-iPr |
| 930 | Cl | OCH₂C₂F₅ | H |
| 931 | Cl | CH₃ | CH₃ |
| 932 | Cl | CH₃ | CF₃ |
| 933 | Cl | CH₃ | OCH₃ |
| 934 | Cl | CH₃ | OCF₃ |
| 935 | Cl | CH₃ | F |
| 936 | Cl | CH₃ | Cl |
| 937 | Cl | CH₃ | Br |
| 938 | Cl | CH₃ | 1-CN-cPr |
| 939 | Cl | CH₃ | 1-CN-iPr |
| 940 | Cl | CH₃ | H |
| 941 | Cl | OCH₃ | CH₃ |
| 942 | Cl | OCH₃ | CF₃ |
| 943 | Cl | OCH₃ | OCH₃ |
| 944 | Cl | OCH₃ | OCF₃ |
| 945 | Cl | OCH₃ | F |
| 946 | Cl | OCH₃ | Cl |
| 947 | Cl | OCH₃ | Br |
| 948 | Cl | OCH₃ | 1-CN-cPr |
| 949 | Cl | OCH₃ | 1-CN-iPr |
| 950 | Cl | OCH₃ | H |
| 951 | Cl | F | CH₃ |
| 952 | Cl | F | CF₃ |
| 953 | Cl | F | OCH₃ |
| 954 | Cl | F | OCF₃ |
| 955 | Cl | F | F |
| 956 | Cl | F | Cl |
| 957 | Cl | F | Br |
| 958 | Cl | F | 1-CN-cPr |
| 959 | Cl | F | 1-CN-iPr |
| 960 | Cl | F | H |
| 961 | Cl | Cl | CH₃ |
| 962 | Cl | Cl | CF₃ |
| 963 | Cl | Cl | OCH₃ |
| 964 | Cl | Cl | OCF₃ |
| 965 | Cl | Cl | F |
| 966 | Cl | Cl | Cl |
| 967 | Cl | Cl | Br |
| 968 | Cl | Cl | 1-CN-cPr |
| 969 | Cl | Cl | 1-CN-iPr |
| 970 | Cl | Cl | H |
| 971 | Cl | Br | CH₃ |
| 972 | Cl | Br | CF₃ |
| 973 | Cl | Br | OCH₃ |
| 974 | Cl | Br | OCF₃ |
| 975 | Cl | Br | F |
| 976 | Cl | Br | Cl |
| 977 | Cl | Br | Br |
| 978 | Cl | Br | 1-CN-cPr |
| 979 | Cl | Br | 1-CN-iPr |
| 980 | Cl | Br | H |
| 981 | Cl | SCF₃ | CH₃ |
| 982 | Cl | SCF₃ | CF₃ |
| 983 | Cl | SCF₃ | OCH₃ |
| 984 | Cl | SCF₃ | OCF₃ |
| 985 | Cl | SCF₃ | F |
| 986 | Cl | SCF₃ | Cl |
| 987 | Cl | SCF₃ | Br |
| 988 | Cl | SCF₃ | 1-CN-cPr |
| 989 | Cl | SCF₃ | 1-CN-iPr |
| 990 | Cl | SCF₃ | H |
| 991 | Cl | H | CH₃ |
| 992 | Cl | H | CF₃ |
| 993 | Cl | H | OCH₃ |
| 994 | Cl | H | OCF₃ |
| 995 | Cl | H | F |
| 996 | Cl | H | Cl |
| 997 | Cl | H | Br |
| 998 | Cl | H | 1-CN-cPr |
| 999 | Cl | H | 1-CN-iPr |
| 1000 | Cl | H | H |
| 1001 | Cl | CF₃ | CH₃ |
| 1002 | Cl | CF₃ | CF₃ |
| 1003 | Cl | CF₃ | OCH₃ |
| 1004 | Cl | CF₃ | OCF₃ |
| 1005 | Cl | CF₃ | F |
| 1006 | Cl | CF₃ | Br |
| 1007 | Cl | CF₃ | 1-CN-cPr |
| 1008 | Cl | CF₃ | 1-CN-iPr |
| 1009 | Cl | CF₃ | Cl |
| 1010 | Cl | CF₃ | H |
| 1011 | Cl | OCF₃ | CH₃ |
| 1012 | Cl | OCF₃ | CF₃ |
| 1013 | Cl | OCF₃ | OCH₃ |
| 1014 | Cl | OCF₃ | OCF₃ |
| 1015 | Cl | OCF₃ | F |
| 1016 | Cl | OCF₃ | Cl |
| 1017 | Cl | OCF₃ | Br |
| 1018 | Cl | OCF₃ | 1-CN-cPr |
| 1019 | Cl | OCF₃ | 1-CN-iPr |
| 1020 | Cl | OCF₃ | H |
| 1021 | Cl | OCH₂CF₃ | CH₃ |
| 1022 | Cl | OCH₂CF₃ | CF₃ |
| 1023 | Cl | OCH₂CF₃ | OCH₃ |
| 1024 | Cl | OCH₂CF₃ | OCF₃ |
| 1025 | Cl | OCH₂CF₃ | F |
| 1026 | Cl | OCH₂CF₃ | Cl |
| 1027 | Cl | OCH₂CF₃ | Br |
| 1028 | Cl | OCH₂CF₃ | 1-CN-cPr |
| 1029 | Cl | OCH₂CF₃ | 1-CN-iPr |
| 1030 | Cl | OCH₂CF₃ | H |
| 1031 | Cl | OCH₂C₂F₅ | CH₃ |
| 1032 | Cl | OCH₂C₂F₅ | CF₃ |
| 1033 | Cl | OCH₂C₂F₅ | OCH₃ |
| 1034 | Cl | OCH₂C₂F₅ | OCF₃ |
| 1035 | Cl | OCH₂C₂F₅ | F |
| 1036 | Cl | OCH₂C₂F₅ | Cl |
| 1037 | Cl | OCH₂C₂F₅ | Br |
| 1038 | Cl | OCH₂C₂F₅ | 1-CN-cPr |
| 1039 | Cl | OCH₂C₂F₅ | 1-CN-iPr |
| 1040 | Cl | OCH₂C₂F₅ | H |
| 1041 | Cl | CH₃ | CH₃ |
| 1042 | Cl | CH₃ | CF₃ |
| 1043 | Cl | CH₃ | OCH₃ |
| 1044 | Cl | CH₃ | OCF₃ |
| 1045 | Cl | CH₃ | F |
| 1046 | Cl | CH₃ | Cl |
| 1047 | Cl | CH₃ | Br |
| 1048 | Cl | CH₃ | 1-CN-cPr |
| 1049 | Cl | CH₃ | 1-CN-iPr |
| 1050 | Cl | CH₃ | H |
| 1051 | Cl | OCH₃ | CH₃ |
| 1052 | Cl | OCH₃ | CF₃ |
| 1053 | Cl | OCH₃ | OCH₃ |
| 1054 | Cl | OCH₃ | OCF₃ |
| 1055 | Cl | OCH₃ | F |
| 1056 | Cl | OCH₃ | Cl |
| 1057 | Cl | OCH₃ | Br |
| 1058 | Cl | OCH₃ | 1-CN-cPr |
| 1059 | Cl | OCH₃ | 1-CN-iPr |
| 1060 | Cl | OCH₃ | H |
| 1061 | Cl | F | CH₃ |
| 1062 | Cl | F | CF₃ |
| 1063 | Cl | F | OCH₃ |
| 1064 | Cl | F | OCF₃ |
| 1065 | Cl | F | F |
| 1066 | Cl | F | Cl |
| 1067 | Cl | F | Br |
| 1068 | Cl | F | 1-CN-cPr |
| 1069 | Cl | F | 1-CN-iPr |
| 1070 | Cl | F | H |
| 1071 | Cl | Cl | CH₃ |
| 1072 | Cl | Cl | CF₃ |
| 1073 | Cl | Cl | OCH₃ |
| 1074 | Cl | Cl | OCF₃ |
| 1075 | Cl | Cl | F |
| 1076 | Cl | Cl | Cl |
| 1077 | Cl | Cl | Br |
| 1078 | Cl | Cl | 1-CN-cPr |
| 1079 | Cl | Cl | 1-CN-iPr |
| 1080 | Cl | Cl | H |
| 1081 | Cl | Br | CH₃ |
| 1082 | Cl | Br | CF₃ |
| 1083 | Cl | Br | OCH₃ |
| 1084 | Cl | Br | OCF₃ |
| 1085 | Cl | Br | F |
| 1086 | Cl | Br | Cl |
| 1087 | Cl | Br | Br |
| 1088 | Cl | Br | 1-CN-cPr |
| 1089 | Cl | Br | 1-CN-iPr |
| 1090 | Cl | Br | H |
| 1091 | Cl | SCF₃ | CH₃ |
| 1092 | Cl | SCF₃ | CF₃ |
| 1093 | Cl | SCF₃ | OCH₃ |
| 1094 | Cl | SCF₃ | OCF₃ |
| 1095 | Cl | SCF₃ | F |
| 1096 | Cl | SCF₃ | Cl |
| 1097 | Cl | SCF₃ | Br |
| 1098 | Cl | SCF₃ | 1-CN-cPr |
| 1099 | Cl | SCF₃ | 1-CN-iPr |
| 1100 | Cl | SCF₃ | H |
| 1101 | SCF₃ | H | CH₃ |
| 1102 | SCF₃ | H | CF₃ |
| 1103 | SCF₃ | H | OCH₃ |
| 1104 | SCF₃ | H | OCF₃ |
| 1105 | SCF₃ | H | F |
| 1106 | SCF₃ | H | Cl |
| 1107 | SCF₃ | H | Br |
| 1108 | SCF₃ | H | 1-CN-cPr |
| 1109 | SCF₃ | H | 1-CN-iPr |
| 1110 | SCF₃ | H | H |
| 1111 | SCF₃ | CF₃ | CH₃ |
| 1112 | SCF₃ | CF₃ | CF₃ |
| 1113 | SCF₃ | CF₃ | OCH₃ |
| 1114 | SCF₃ | CF₃ | OCF₃ |
| 1115 | SCF₃ | CF₃ | F |
| 1116 | SCF₃ | CF₃ | Br |
| 1117 | SCF₃ | CF₃ | 1-CN-cPr |
| 1118 | SCF₃ | CF₃ | 1-CN-iPr |
| 1119 | SCF₃ | CF₃ | Cl |
| 1120 | SCF₃ | CF₃ | H |
| 1121 | SCF₃ | OCF₃ | CH₃ |
| 1122 | SCF₃ | OCF₃ | CF₃ |
| 1123 | SCF₃ | OCF₃ | OCH₃ |
| 1124 | SCF₃ | OCF₃ | OCF₃ |
| 1125 | SCF₃ | OCF₃ | F |
| 1126 | SCF₃ | OCF₃ | Cl |
| 1127 | SCF₃ | OCF₃ | Br |
| 1128 | SCF₃ | OCF₃ | 1-CN-cPr |
| 1129 | SCF₃ | OCF₃ | 1-CN-iPr |
| 1130 | SCF₃ | OCF₃ | H |
| 1131 | SCF₃ | OCH₂CF₃ | CH₃ |
| 1132 | SCF₃ | OCH₂CF₃ | CF₃ |
| 1133 | SCF₃ | OCH₂CF₃ | OCH₃ |
| 1134 | SCF₃ | OCH₂CF₃ | OCF₃ |
| 1135 | SCF₃ | OCH₂CF₃ | F |
| 1136 | SCF₃ | OCH₂CF₃ | Cl |
| 1137 | SCF₃ | OCH₂CF₃ | Br |
| 1138 | SCF₃ | OCH₂CF₃ | 1-CN-cPr |
| 1139 | SCF₃ | OCH₂CF₃ | 1-CN-iPr |
| 1140 | SCF₃ | OCH₂CF₃ | H |
| 1141 | SCF₃ | OCH₂C₂F₅ | CH₃ |
| 1142 | SCF₃ | OCH₂C₂F₅ | CF₃ |
| 1143 | SCF₃ | OCH₂C₂F₅ | OCH₃ |
| 1144 | SCF₃ | OCH₂C₂F₅ | OCF₃ |
| 1145 | SCF₃ | OCH₂C₂F₅ | F |
| 1146 | SCF₃ | OCH₂C₂F₅ | Cl |
| 1147 | SCF₃ | OCH₂C₂F₅ | Br |
| 1148 | SCF₃ | OCH₂C₂F₅ | 1-CN-cPr |
| 1149 | SCF₃ | OCH₂C₂F₅ | 1-CN-iPr |
| 1150 | SCF₃ | OCH₂C₂F₅ | H |
| 1151 | SCF₃ | CH₃ | CH₃ |
| 1152 | SCF₃ | CH₃ | CF₃ |
| 1153 | SCF₃ | CH₃ | OCH₃ |
| 1154 | SCF₃ | CH₃ | OCF₃ |
| 1155 | SCF₃ | CH₃ | F |
| 1156 | SCF₃ | CH₃ | Cl |
| 1157 | SCF₃ | CH₃ | Br |
| 1158 | SCF₃ | CH₃ | 1-CN-cPr |
| 1159 | SCF₃ | CH₃ | 1-CN-iPr |
| 1160 | SCF₃ | CH₃ | H |
| 1161 | SCF₃ | OCH₃ | CH₃ |
| 1162 | SCF₃ | OCH₃ | CF₃ |
| 1163 | SCF₃ | OCH₃ | OCH₃ |
| 1164 | SCF₃ | OCH₃ | OCF₃ |
| 1165 | SCF₃ | OCH₃ | F |
| 1166 | SCF₃ | OCH₃ | Cl |
| 1167 | SCF₃ | OCH₃ | Br |
| 1168 | SCF₃ | OCH₃ | 1-CN-cPr |
| 1169 | SCF₃ | OCH₃ | 1-CN-iPr |
| 1170 | SCF₃ | OCH₃ | H |
| 1171 | SCF₃ | F | CH₃ |
| 1172 | SCF₃ | F | CF₃ |
| 1173 | SCF₃ | F | OCH₃ |
| 1174 | SCF₃ | F | OCF₃ |
| 1175 | SCF₃ | F | F |
| 1176 | SCF₃ | F | Cl |
| 1177 | SCF₃ | F | Br |
| 1178 | SCF₃ | F | 1-CN-cPr |
| 1179 | SCF₃ | F | 1-CN-iPr |
| 1180 | SCF₃ | F | H |
| 1181 | SCF₃ | Cl | CH₃ |
| 1182 | SCF₃ | Cl | CF₃ |
| 1183 | SCF₃ | Cl | OCH₃ |
| 1184 | SCF₃ | Cl | OCF₃ |
| 1185 | SCF₃ | Cl | F |
| 1186 | SCF₃ | Cl | Cl |
| 1187 | SCF₃ | Cl | Br |
| 1188 | SCF₃ | Cl | 1-CN-cPr |
| 1189 | SCF₃ | Cl | 1-CN-iPr |
| 1190 | SCF₃ | Cl | H |
| 1191 | SCF₃ | Br | CH₃ |
| 1192 | SCF₃ | Br | CF₃ |
| 1193 | SCF₃ | Br | OCH₃ |
| 1194 | SCF₃ | Br | OCF₃ |
| 1195 | SCF₃ | Br | F |
| 1196 | SCF₃ | Br | Cl |
| 1197 | SCF₃ | Br | Br |
| 1198 | SCF₃ | Br | 1-CN-cPr |
| 1199 | SCF₃ | Br | 1-CN-iPr |
| 1200 | SCF₃ | Br | H |
| 1201 | SCF₃ | SCF₃ | CH₃ |
| 1202 | SCF₃ | SCF₃ | CF₃ |
| 1203 | SCF₃ | SCF₃ | OCH₃ |
| 1204 | SCF₃ | SCF₃ | OCF₃ |
| 1205 | SCF₃ | SCF₃ | F |
| 1206 | SCF₃ | SCF₃ | Cl |
| 1207 | SCF₃ | SCF₃ | Br |
| 1208 | SCF₃ | SCF₃ | 1-CN-cPr |
| 1209 | SCF₃ | SCF₃ | 1-CN-iPr |
| 1210 | SCF₃ | SCF₃ | H |

The invention also relates to a mixture of at least one compound of the invention with at least one mixing partner. Preferred are binary mixtures of one compound of the invention as component I with one mixing partner herein as component II. Preferred weight ratios for such binary mixtures are from 5000:1 to 1:5000, preferably from 1000:1 to 1:1000, more preferably from 100:1 to 1:100, particularly from 10:1 to 1:10. In such binary mixtures, components I and II may be used in equal amounts, or an excess of component I, or an excess of component II may be used.

Mixing partners can be selected from pesticides, in particular insecticides, nematicides, and acaricides, fungicides, herbicides, plant growth regulators, fertilizers. Preferred mixing partners are insecticides, nematicides, and fungicides.

The invention also relates to agrochemical compositions comprising an auxiliary and at least one compound of formula (I).

An agrochemical composition comprises a pesticidally effective amount of a compound of formula (I).

The compounds of formula (I) can be converted into customary types of agro-chemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials e.g. seeds (e.g. GF). These and further compositions types are defined in the “Catalogue of pesticide formulation types and international coding system”, Technical Monograph No. 2, 6th Ed. May 2008, CropLife International. The compositions are prepared in a known manner, e.g. described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents. Suitable solid carriers or fillers are mineral earths.

Suitable surfactants are surface-active compounds, e.g. anionic, cationic, nonionic, and amphoteric surfactants, block polymers, polyelectrolytes. Such surfactants can be used as emulsifier, dispersant, solubilizer, wetter, penetration enhancer, protective colloid, or adjuvant. Surfactants are listed in McCutcheon's, Vol. 1: Emulsifiers & Detergents, McCutcheon's Directories, Glen Rock, USA, 2008 (International or North American Ed.). Suitable anionic surfactants are alkali, alkaline earth, or ammonium salts of sulfonates, sulfates, phosphates, carboxylates.

Suitable nonionic surfactants are alkoxylates, N-substituted fatty acid amides, amine oxides, esters, sugar-based surfactants, polymeric surfactants. Suitable cationic surfactants are quaternary surfactants.

The agrochemical compositions generally comprise between 0.01 and 95%, preferably between 0.1 and 90%, and most preferably between 0.5 and 75%, by weight of active substance.

The active substances are employed in a purity of from 90% to 100%, preferably from 95% to 100%.

Various types of oils, wetters, adjuvants, or fertilizer may be added to the active substances or the compositions comprising them as premix or, if appropriate not until immediately prior to use (tank mix). These agents can be admixed with the compositions according to the invention in a weight ratio of 1:100 to 100:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, of the ready-to-use spray liquor are applied per hectare of agricultural useful area.

The compounds of formula (I) are suitable for use in protecting crops, plants, plant propagation materials, e.g. seeds, or soil or water, in which the plants are growing, from attack or infestation by animal pests. Therefore, the invention also relates to a plant protection method, which comprises contacting crops, plants, plant propagation materials, e.g. seeds, or soil or water, in which the plants are growing, to be protected from attack or infestation by animal pests, with a pesticidally effective amount of a compound of formula (I).

The compounds of formula (I) are also suitable for use in combating or controlling animal pests. There-fore, the invention also relates to a method of combating or controlling animal pests, which comprises contacting the animal pests, their habitat, breeding ground, or food supply, or the crops, plants, plant propagation materials, e.g. seeds, or soil, or the area, material or environment in which the animal pests are growing or may grow, with a pesticidally effective amount of a compound of formula (I).

The compounds of formula (I) are effective through both contact and ingestion to any and all developmental stages, such as egg, larva, pupa, and adult.

The compounds of formula (I) can be applied as such or in form of compositions comprising them.

The application can be carried out both before and after the infestation of the crops, plants, plant propagation materials by the pests.

The term “contacting” includes both direct contact (applying the compounds/compositions directly on the animal pest or plant) and indirect contact (applying the compounds/compositions to the locus).

The term “animal pest” includes arthropods, gastropods, and nematodes. Preferred animal pests according to the invention are arthropods, preferably insects and arachnids, in particular insects.

The term “plant” includes cereals, e.g. durum and other wheat, rye, barley, triticale, oats, rice, or maize (fodder maize and sugar maize/sweet and field corn); beet, e.g. sugar beet, or fodder beet; fruits, e.g. pomes, stone fruits, or soft fruits, e.g. apples, pears, plums, peaches, nectarines, almonds, cherries, papayas, strawberries, raspberries, blackberries or gooseberries; leguminous plants, e.g. beans, lentils, peas, alfalfa, or soybeans; oil plants, e.g. rapeseed (oilseed rape), turnip rape, mustard, olives, sunflowers, coconut, cocoa beans, castor oil plants, oil palms, ground nuts, or soybeans; cucurbits, e.g. squashes, pumpkins, cucumber or melons; fiber plants, e.g. cotton, flax, hemp, or jute; citrus fruit, e.g. oranges, lemons, grape-fruits or mandarins; vegetables, e.g. eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, e.g. avocados, cinnamon, or camphor; energy and raw material plants, e.g. corn, soybean, rapeseed, sugar cane or oil palm; tobacco; nuts, e.g. walnuts; pistachios; coffee; tea; bananas; vines; hop; sweet leaf (Stevia); natural rubber plants or ornamental and forestry plants, shrubs, broad-leaved trees or evergreens, eucalyptus; turf; lawn; grass. Preferred plants include potatoes sugar beets, tobacco, wheat, rye, barley, oats, rice, corn, cotton, soybeans, rapeseed, legumes, sunflowers, coffee, or sugar cane; fruits; vines; ornamentals; or vegetables, e.g. cucumbers, tomatoes, beans or squashes.

The term “seed” embraces seeds and plant propagules including true seeds, seed pieces, suckers, corms, bulbs, fruit, tubers, grains, cuttings, cut shoots, and means preferably true seeds.

“Pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions e.g. desired pesticidal effect and duration, weather, target species, locus, mode of application.

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare.

The compounds of formula (I) are also suitable for use against non-crop insect pests. For use against said non-crop pests, compounds of formula (I) can be used as bait composition, gel, general insect spray, aero-sol, as ultra-low volume application and bed net (impregnated or surface applied).

The term “non-crop insect pest” refers to pests, which are particularly relevant for non-crop targets, e.g. ants, termites, wasps, flies, ticks, mosquitoes, bed bugs, crickets, or cockroaches, such as: Aedes aegypti, Musca domestica, Tribolium spp.; termites such as Reticulitermes flavipes, Coptotermes formosanus; roaches such as Blatella germanica, Periplaneta americana; ants such as Solenopsis invicta, Linepithema humile, and Camponotus pennsylvanicus.

The bait can be a liquid, a solid or a semisolid preparation (e.g. a gel). For use in bait compositions, the typical content of active ingredient is from 0.001 wt % to 15 wt %, desirably from 0.001 wt % to 5 wt % of active compound.

The compounds of formula (I) and its compositions can be used for protecting wooden materials such as trees, board fences, sleepers, frames, artistic artifacts, etc. and buildings, but also construction materials, furniture, leathers, fibers, vinyl articles, electric wires and cables etc. from ants, termites and/or wood or textile destroying beetles, and for controlling ants and termites from doing harm to crops or human beings (e.g. when the pests invade into houses and public facilities or nest in yards, orchards or parks).

Customary application rates in the protection of materials are, e.g., from 0.001 g to 2000 g or from 0.01 g to 1000 g of active compound per m² treated material, desirably from 0.1 g to 50 g per m².

Insecticidal compositions for use in the impregnation of materials typically contain from 0.001 to 95 wt %, preferably from 0.1 to 45 wt %, and more preferably from 1 to 25 wt % of at least one repellent and/or insecticide.

Pests

The compounds of the invention are especially suitable for efficiently combating animal pests e.g. arthropods, and nematodes including:

insects from the sub-order of Auchenorrhyncha, e.g. Amrasca biguttula, Empoasca spp., Nephotettix virescens, Sogatella furcifera, Mahanarva spp., Laodelphax striatellus, Nilaparvata lugens, Diaphorina citri;

Lepidoptera, e.g. Helicoverpa spp., Heliothis virescens, Lobesia botrana, Ostrinia nubilalis, Plutella xylostella, Pseudoplusia includens, Scirpophaga incertulas, Spodoptera spp., Trichoplusia ni, Tuta absoluta, Cnaphalocrocis medialis, Cydia pomonella, Chilo suppressalis, Anticarsia gemmatalis, Agrotis ipsilon, Chrysodeixis includens;

True bugs, e.g. Lygus spp., Stink bugs such as Euschistus spp., Halyomorpha halys, Nezara viridula, Piezodorus guildinii, Dichelops furcatus;

Thrips, e.g. Frankliniella spp., Thrips spp., Dichromothrips corbettii;

Aphids, e.g. Acyrthosiphon pisum, Aphis spp., Myzus persicae, Rhopalosiphum spp., Schizaphis graminum, Megoura viciae;

Whiteflies, e.g. Trialeurodes vaporariorum, Bemisia spp.;

Coleoptera, e.g. Phyllotreta spp., Melanotus spp., Meligethes aeneus, Leptinotarsa decimlineata, Ceutorhynchus spp., Diabrotica spp., Anthonomus grandis, Atomaria linearia, Agriotes spp., Epilachna spp.;

Flies, e.g. Delia spp., Ceratitis capitate, Bactrocera spp., Liriomyza spp.;

Coccoidea, e.g. Aonidiella aurantia, Ferrisia virgate;

Anthropods of class Arachnida (Mites), e.g. Penthaleus major, Tetranychus spp.;

Nematodes, e.g. Heterodera glycines, Meloidogyne spp., Pratylenchus spp., Caenorhabditis elegans.

Animal Health

The compounds of formula (I) are suitable for use in treating or protecting animals against infestation or infection by parasites. Therefore, the invention also relates to the use of a compound of the invention for the manufacture of a medicament for the treatment or protection of animals against infestation or infection by parasites. Furthermore, the invention relates to a method of treating or protecting animals against infestation and infection by parasites, which comprises orally, topically or parenterally administering or applying to the animals a parasiticidally effective amount of a compound of formula (I).

The invention also relates to the non-therapeutic use of compounds of the invention for treating or protecting animals against infestation and infection by parasites. Moreover, the invention relates to a non-therapeutic method of treating or protecting animals against infestation and infection by parasites, which comprises applying to a locus a parasiticidally effective amount of a compound of formula (I).

The compounds of the invention are further suitable for use in combating or controlling parasites in and on animals. Furthermore, the invention relates to a method of combating or controlling parasites in and on animals, which comprises contacting the parasites with a parasitically effective amount of a compound of formula (I).

The invention also relates to the non-therapeutic use of compounds of formula (I) for controlling or combating parasites. Moreover, the invention relates to a non-therapeutic method of combating or controlling parasites, which comprises applying to a locus a parasiticidally effective amount of a compound of formula (I).

The compounds of formula (I) can be effective through both contact (via soil, glass, wall, bed net, carpet, blankets or animal parts) and ingestion (e.g. baits). Furthermore, the compounds of formula (I) can be applied to any and all developmental stages.

The compounds of formula (I) can be applied as such or in form of compositions comprising them.

The term “locus” means the habitat, food supply, breeding ground, area, material or environment in which a parasite is growing or may grow outside of the animal.

As used herein, the term “parasites” includes endo- and ectoparasites. In some embodiments of the invention, endoparasites can be preferred. In other embodiments, ectoparasites can be preferred. Infestations in warm-blooded animals and fish include lice, biting lice, ticks, nasal bots, keds, biting flies, muscoid flies, flies, myiasitic fly larvae, chiggers, gnats, mosquitoes and fleas.

The compounds of the invention are especially useful for combating the following parasites: Cimex lectularius, Rhipicephalus sanguineus, and Ctenocephalides felis.

As used herein, the term “animal” includes warm-blooded animals (including humans) and fish. Preferred are mammals, such as cattle, sheep, swine, camels, deer, horses, pigs, poultry, rabbits, goats, dogs and cats, water buffalo, donkeys, fallow deer and reindeer, and also in furbearing animals such as mink, chinchilla and raccoon, birds such as hens, geese, turkeys and ducks and fish such as fresh- and salt-water fish such as trout, carp and eels. Particularly preferred are domestic animals, such as dogs or cats.

The compounds of formula (I) may be applied in total amounts of 0.5 mg/kg to 100 mg/kg per day, preferably 1 mg/kg to 50 mg/kg per day.

For oral administration to warm-blooded animals, the compounds of formula (I) may be formula ted as animal feeds, animal feed premixes, animal feed concentrates, pills, solutions, pastes, suspensions, drenches, gels, tablets, boluses and capsules. For oral administration, the dosage form chosen should provide the animal with 0.01 mg/kg to 100 mg/kg of animal body weight per day of the compounds of formula (I), preferably with 0.5 mg/kg to 100 mg/kg of animal body weight per day.

Alternatively, the compounds of formula (I) may be administered to animals parenterally, e.g., by intraruminal, intramuscular, intravenous or subcutaneous injection. The compounds of formula (I) may be dispersed or dissolved in a physiologically acceptable carrier for subcutaneous injection. Alternatively, the compounds of formula (I) may be formulated into an implant for subcutaneous administration. In addition the compounds of formula (I) may be transdermally administered to animals. For parenteral administration, the dosage form chosen should provide the animal with 0.01 mg/kg to 100 mg/kg of animal body weight per day of the compounds of formula (I).

The compounds of formula (I) may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compounds of formula (I). In addition, the compounds of formula (I) may be formulated as ear tags for animals, particularly quadrupeds e.g. cattle and sheep.

Oral solutions are administered directly.

Solutions for use on the skin are trickled on, spread on, rubbed in, sprinkled on or sprayed on.

Gels are applied to or spread on the skin or introduced into body cavities.

Pour-on formulations are poured or sprayed onto limited areas of the skin, the active compound penetrating the skin and acting systemically. Pour-on formulations are prepared by dissolving, suspending or emulsifying the active compound in suitable skin-compatible solvents or solvent mixtures.

Emulsions can be administered orally, dermally or as injections.

Suspensions can be administered orally or topically/dermally.

Semi-solid preparations can be administered orally or topically/dermally.

For the production of solid preparations, the active compound is mixed with suitable excipients, if appropriate with addition of auxiliaries, and brought into the desired form.

The compositions which can be used in the invention can comprise generally from about 0.001 to 95% of the compound of formula (I).

Ready-to-use preparations contain the compounds acting against parasites, preferably ectoparasites, in concentrations of 10 ppm to 80% by weight, preferably from 0.1 to 65% by weight, more preferably from 1 to 50% by weight, most preferably from 5 to 40% by weight.

Preparations which are diluted before use contain the compounds acting against ectoparasites in concentrations of 0.5 to 90% by weight, preferably of 1 to 50% by weight.

Furthermore, the preparations comprise the compounds of formula I against endoparasites in concentrations of 10 ppm to 2% by weight, preferably of 0.05 to 0.9% by weight, very particularly preferably of 0.005 to 0.25% by weight.

Solid formulations which release compounds of the invention may be applied in total amounts of 10 mg/kg to 300 mg/kg, preferably 20 mg/kg to 200 mg/kg, most preferably 25 mg/kg to 160 mg/kg body weight of the treated animal in the course of three weeks.

The following examples illustrate the invention.

### A. PREPARATION OF COMPOUNDS

Materials: Unless otherwise noted, reagents and solvents were purchased at highest commercial quality and used without further purification. Dry tetrahydrofuran (THF), ethylacetate (EtOAc), dimethylsulfoxide (DMSO), acetone, ethanol (EtOH), benzene, dimethylformamide, (DMF), diisopropylethylamine (DIPEA), hexafluorophosphate azabenzotriazole tetramethyl uronium (HATU), pyridine, and CH2Cl2 were purchased from commercial providers.

All reactions were monitored by thin-layer chromatography (TLC) using Merck silica gel 60 F₂54 pre-coated plates (0.25 mm). Flash chromatography was carried out with Kanto Chemical silica gel (Kanto Chemical, silica gel 60N, spherical neutral, 0.040-0.050 mm, Cat.-No. 37563-84). ¹H NMR spectra were recorded on JEOL JNM-ECA-500 (500 MHz). Chemical shifts are expressed in ppm downfield from the internal solvent peaks for acetone-d₆ (¹H; δ=2.05 ppm) and CD₃OD (¹H; δ=3.30 ppm), and J values are given in Hertz. The following abbreviations were used to explain the multiplicities: s=singlet, d=doublet, t=triplet, q=quartet, dd=double doublet, dt=double triplet, m=multiplet, br=broad. High-resolution mass spectra were measured on a JEOL JMS-T100LP.

Characterization: The compounds were characterized by coupled High Performance Liquid Chromatography with mass spectrometry (HPLC/MS). Method A: UHPLC-MS on Shimadzu Nexera UHPLC & Shimadzu LCMS 20-20 ESI. Analytical UHPLC column: Phenomenex Kinetex 1.7 μm XB-C18 100A; 50×2.1 mm; mobile phase: A: water+0.1% TFA; B: acetonitrile; gradient: 5-100% B in 1.50 minutes; 100% B 0.20 min; flow: 0.8-1.0 mL/min in 1.50 minutes at 60° C. MS-method: ESI positive; mass range (m/z) 100-700. M+1 means mass of the molecule plus 1 Dalton.

### Synthesis Example A

### Example 1: 2-(3-ethylsulfonylimidazo[1,2-a]pyridin-2-yl)-1-methyl-6-(trifluoromethoxy)imidazo[4,5-c]quinoline (compound C-4)

### Step 1: Synthesis of N-methyl-3-nitro-8-(trifluoromethoxy)quinolin-4-amine

To a solution of 4-chloro-3-nitro-8-(trifluoromethoxy)quinoline (4 g) in THF (40 mL), at 20 to 25° C., was added methylamine (40 mL, 2M solution in THF). The reaction mixture was then warmed to 50° C. and stirred for 1 h. Reaction was monitored by TLC, after the complete conversion of 4-chloro-3-nitro-8-(trifluoromethoxy)quinoline, the reaction mixture was then concentrated in vacuo, to afford a residue containing N-methyl-3-nitro-8-(trifluoromethoxy)quinolin-4-amine (3.9 g, 100% yield), which was used in Step 2 without further purification. Similar procedure is described in WO 2008117225. HPLC-MS (Method A): mass found for C₁₁HF₃N₃O₃ [M+H]+ 287.8; tR=0.791 min.

### Step 2: Synthesis of N4-methyl-8-(trifluoromethoxy)quinoline-3,4-diamine

To a suspension of Zn-powder (3.6 g) in CH₃COOH (60 mL) was slowly added a solution of N-methyl-3-nitro-8-(trifluoromethoxy)quinolin-4-amine (3.9 g) in 10 mL EtOAc at a temperature of up to 30° C. The reaction mixture was stirred for an additional 2 h at 20 to 25° C. After the complete conversion of N-methyl-3-nitro-8-(trifluoromethoxy)quinolin-4-amine, the reaction mixture was diluted with EtOAc and filtrated. The filtrate was washed with H₂O. The combined H₂O-phases were adjusted to an alkaline pH with aqueous NaOH and extracted with EtOAc. The combined organic extracts were dried and concentrated in vacuo to afford a residue containing N4-methyl-8-(trifluoromethoxy)quinoline-3,4-diamine (2.35 g, 67% yield), which was used in Step 3 without further purification. HPLC-MS (Method A): mass found for C₁₁H₁₀F₃N₃O [M+H]+ 257.8; tR=0.665 min.

### Step 3: Synthesis of 3-ethylsulfanyl-N-[4-(methylamino)-8-(trifluoromethoxy)-3-quinolyl]imidazo[1,2-a]pyridine-2-carboxamide

To a stirred solution of N4-methyl-8-(trifluoromethoxy)quinoline-3,4-diamine (0.417 g, 0.0016 mol) in DMF (15 V) at 0° C., DIPEA (0.34 g, 0.003 mol) and 3-ethylsulfanylimidazo[1,2-a]pyridine-2-carboxylic acid (was synthesised similarly as mentioned in WO2016162318) (0.30 g, 0.0013 mol) were added, then was followed by the addition of HATU (0.82 g, 0.002 mol) portion wise. The resultant reaction mixture was stirred at the room temperature for 24 h. Reaction was monitored by TLC, after the complete conversion of starting material, reaction mixture was partitioned between ethyl acetate (150 mL×2) and water (250 mL×2). Organic layer was separated, dried over Na₂SO₄ and concentrated to get crude mass. Crude was purified by column chromatography eluting with 20% ethyl acetate in heptane gradient to afford 3-ethylsulfanyl-N-[4-(methylamino)-8-(trifluoromethoxy)-3-quinolyl]imidazo[1,2-a]pyridine-2-carboxamide as an off white solid. (0.60 g, 95% yield). LC-MS: mass calculated for C₂₁H₁₈F₃N₅O₂S [M+H]⁺ 462.0, found 462.0; Rₜ=0.867 min (Rₜ: retention time).

### Step 4: Synthesis of 2-(3-ethylsulfanylimidazo[1,2-a]pyridin-2-yl)-1-methyl-6-(trifluoromethoxy)imidazo[4,5-c]quinoline

A suspension of 3-ethylsulfanyl-N-[4-(methylamino)-8-(trifluoromethoxy)-3-quinolyl]imidazo[1,2-a]pyridine-2-carboxamide (0.21 g, 0.46 mmol) in acetic acid (3 V) was refluxed for 5 h. Reaction was monitored by HPLC, after the complete conversion of starting material, reaction mixture was partitioned between ethyl acetate (150 mL×2) and water (250 mL×2). Organic layer was separated, washed with saturated bicarbonate solution (100 mL×2). The combined organic layers were separated, dried over Na₂SO₄ and concentrated to get crude mass. Crude was purified by column chromatography eluting with 10% ethyl acetate in heptane gradient to afford 2-(3-ethylsulfanylimidazo[1,2-a]pyridin-2-yl)-1-methyl-6-(trifluoromethoxy)imidazo[4,5-c]quinoline as an off white solid. (0.14 g, 67% yield). LC-MS: mass calculated for C₂₁H₁₆F₃N₅OS [M+H]⁺ 444.0, found 444.0; Rₜ=1.013 min (Rₜ: retention time).

### Step 5: Synthesis of 2-(3-ethylsulfonylimidazo[1,2-a]pyridin-2-yl)-1-methyl-6-(trifluoromethoxy)imidazo[4,5-c]quinoline

A suspension of 2-(3-ethylsulfanylimidazo[1,2-a]pyridin-2-yl)-1-methyl-6-(trifluoromethoxy)imidazo[4,5-c]quinoline (139 mg, 0.31 mmol) in acetic acid (3 mL) was stirred at RT. Then to the reaction mixture Na₂WO₄.H₂O (3 mg, 0.0094 mmol) and 30% H₂O₂ (89 μL) was added and the reaction was allowed to stir at RT overnight. Reaction was monitored by HPLC, after the complete conversion of starting material, reaction mixture was completely evaporated on rotavapor. The reaction mixture was dissolved in Ethyl acetate (15 mL) and washed with saturated bicarbonate solution (20 mL×2). The combined organic layers were separated, dried over Na₂SO₄ and concentrated to get crude mass. Crude was purified by column chromatography eluting with 10% ethyl acetate in heptane gradient to afford 2-(3-ethylsulfonylimidazo[1,2-a]pyridin-2-yl)-1-methyl-6-(trifluoromethoxy)imidazo[4,5-c]quinoline as an off white solid. (75 mg, 50.7% yield). LC-MS: mass calculated for C₂₁H₁₆F₃N₅O₃S [M+H]⁺ 476.0, found 476.0; Rₜ=0.966 min (Rₜ: retention time).

### Example 2: 8-(3-ethylsulfonylimidazo[1,2-a]pyridin-2-yl)-4-(trifluoromethyl)imidazo[1,2-a][1,8]naphthyridine (compound C-7)

### Step 1: Synthesis of N-[7-hydroxy-5-(trifluoromethyl)-1,8-naphthyridin-2-yl]acetamide

A suspension of 7-amino-4-(trifluoromethyl)-1,8-naphthyridin-2-ol (4 g, 0.017 mol) in acetic anhydride (10 V) was refluxed to 2 h. Reaction was monitored by HPLC, after the complete conversion of 7-amino-4-(trifluoromethyl)-1,8-naphthyridin-2-ol, the above reaction mixture was cooled to room temperature, obtained solid was filtered and washed with water (100×2). Solid was dried over rota to afford desired compound 2 as a brown solid. (3.9 g, 83% yield). The above reaction was followed by the literature Organic & Biomolecular Chemistry Volume 10. LC-MS: mass calculated for C₁₁H₈H₃N₃O₂ [M+H]⁺ 272.0, found 271.9; Rₜ=0.760 min (Rₜ: retention time).

### Step 2: Synthesis of N-[7-chloro-5-(trifluoromethyl)-1,8-naphthyridin-2-yl]acetamide

A suspension of N-[7-hydroxy-5-(trifluoromethyl)-1,8-naphthyridin-2-yl]acetamide (3.9 g, 0.014 mol) in POCl₃ (10 V) at 0° C., then the resultant reaction mixture was gradually heated to 100° C. for 90 minutes. Reaction was monitored by HPLC, after the complete conversion of SM, the above reaction mixture was cooled to room temperature, quenched with water (200 mL) maintaining the exothermicity of reaction mixture. Then, was followed by the addition of 10% ammonia solution until pH 9. Obtained solid was filtered and washed with water (100×2). Solid was dried over rota to afford desired N-[7-chloro-5-(trifluoromethyl)-1,8-naphthyridin-2-yl]acetamide as a brown solid. (3.9 g, 95% yield). The above reaction was followed by literature as Journal of the American Chemical Society Volume 123. LC-MS: mass calculated for C₁₁H₇ClF₃N₃O [M+H]⁺ 290.0, found 289.7; Rₜ=1.001 min (Rₜ: retention time).

### Step 3: Synthesis of 7-chloro-5-(trifluoromethyl)-1,8-naphthyridin-2-amine

A suspension of N-[7-chloro-5-(trifluoromethyl)-1,8-naphthyridin-2-yl]acetamide (3.9 g, 0.013 mol) in 10% sulphuric acid (20 V) was refluxed for 2 h. Reaction was monitored by HPLC, after the complete conversion of SM, the above reaction mixture was cooled to room temperature, quenched with water (200 mL) maintaining the exothermicity of reaction mixture. Then, was followed by the addition of 10% ammonia solution until pH 9. Obtained solid was filtered and washed with water (100×2). Solid was dried over rota to afford desired 7-chloro-5-(trifluoromethyl)-1,8-naphthyridin-2-amine as a yellow solid. (3.5 g, 90% yield). The above reaction was followed by literature as WO 2016210234 A1. LC-MS: mass calculated for C₉H₅ClF₃N₃[M+H]⁺ 248.0, found 247.8; Rₜ=0.759 min (Rₜ: retention time).

### Step 4: Synthesis of 2-chloro-8-(3-ethylsulfonylimidazo[1,2-a]pyridin-2-yl)-4-(trifluoromethyl)imidazo[1,2-a][1,8]naphthyridine

To a stirred solution of 7-chloro-5-(trifluoromethyl)-1,8-naphthyridin-2-amine (1 g, 0.004 mol) in tert-butanol (10 V) was added 2-bromo-1-(3-ethylsulfonylimidazo[1,2-a]pyridin-2-yl)ethanone (synthesised as described in WO2016129684 A1) (1.34 g, 0.004 mol) and the resultant reaction mixture was heated in Radley's to 95° C. for 5 days. Reaction was monitored by TLC, after the complete conversion of SM, the above reaction mixture was filtered through celite bed, celite bed was washed with ethyl acetate (30 mL×3), filtrate was collected and concentrated under reduced pressure to get crude mass. Crude was purified by column chromatography eluting 40% with ethyl acetate in heptane gradient to afford 2-chloro-8-(3-ethylsulfonylimidazo[1,2-a]pyridin-2-yl)-4-(trifluoromethyl)imidazo[1,2-a][1,8]naphthyridine as a brown solid (0.5 g, 34% yield). The compound was synthesized using similar procedure as described in WO 2017/167832. LC-MS: mass calculated for C₂₀H₁₃ClF₃N₅O₂S [M+H]⁺ 480.0, found 480.0; Rₜ=1.031 min (Rₜ: retention time).

### Step 5: Synthesis of 8-(3-ethylsulfonylimidazo[1,2-a]pyridin-2-yl)-4-(trifluoromethyl) imidazo[1,2-a][1,8]naphthyridine

To a stirred solution of 2-chloro-8-(3-ethylsulfonylimidazo[1,2-a]pyridin-2-yl)-4-(trifluoromethyl)imidazo[1,2-a][1,8]naphthyridine (0.5 g, 0.001 mol) in methanol (5 V), were added cyclohexene (0.34 g, 0.004 mol) and Pd 10% on activated carbon (0.106 g, 0.1 mmol) in microwave at 90° C. for 30 minutes. Reaction was monitored by HPLC, after the complete conversion of SM, reaction mixture was filtered through celite bed, celite bed was washed with ethyl acetate (30 mL×3). Filtrate was concentrated on rota and the residue was subjected to purification by column chromatography eluting with 10% ethyl acetate in heptane gradient to afford desired compound as an off white solid. (0.16 g, 37% yield). LC-MS: mass calculated for C₂₀H₁₄F₃N₅O₂ [M+H]⁺ 446.0, found 446.0; Rₜ=1.008 min (Rₜ: retention time).

### Example 3: 2-(3-ethylsulfonylimidazo[1,2-a]pyridin-2-yl)-1-methyl-5-(trifluoromethyl)imidazo[4,5-f]quinoline (compound C-11)

### Step 1: synthesis of N-[6-nitro-8-(trifluoromethyl)-5-quinolyl]acetamide

To a solution of 6-nitro-8-(trifluoromethyl)quinolin-5-amine (10.03 mmol) and (CH₃CH₂)₃N (30.1 mmol) in THF (25 ml) at 20 to 25° C. was added acetylacetate (50.16 mmol) dropwise. The resulting reaction mixture was stirred at 20 to 25° C. for 7 days. Then, (CH₃CH₂)₃N (10.03 mmol) and acetyl acetate (20.06 mmol) were added and the reaction mixture, which was subsequently stirred for another 7 days. The reaction mixture was then concentrated under reduced pressure to afford a residue. The residue was dissolved in H₂O, and extracted. The organic layer was dried, filtered and concentrated under reduced pressure to afford N-[6-nitro-8-(trifluoromethyl)-5-quinolyl]acetamide (2.97 g) The crude product was used in the next step without further purification. LC/MS retention time: 1.048 min, m/z=300 (M+H⁺)

### Step 2: synthesis N-methyl-N-[6-nitro-8-(trifluoromethyl)-5-quinolyl]acetamide

To a solution of N-[6-nitro-8-(trifluoromethyl)-5-quinolyl]acetamide (9.93 mmol) in DMF (40 ml) at 20 to 25° C. was added Cs₂CO₃ (29.78 mmol). The reaction mixture was then cooled to 0° C. and iodomethane (14.89 mmol) was added dropwise. The resulting mixture was allowed to warm up to 20 to 25° C. and stirred for 12-16 hours. The reaction mixture was then concentrated under reduced pressure to afford a residue. The residue was dissolved in CH₂Cl₂ and washed with H₂O. The organic layer was dried, filtered and concentrated under reduced pressure to afford N-methyl-N-[6-nitro-8-(trifluoromethyl)-5-quinolyl]acetamide (2.85 g). The crude product was used in the next step without further purification. LC/MS retention time: 0.962 min, m/z=314 (M+H⁺)

### Step 3: synthesise of N-methyl-6-nitro-8-(trifluoromethyl)quinolin-5-amine

To a solution of N-methyl-N-[6-nitro-8-(trifluoromethyl)-5-quinolyl]acetamide (9.10 mmol) in CH₃COOH (conc., 25 ml) at 20 to 25° C. was added sulfuric acid (conc., 3.5 ml). The resulting reaction mixture was heated to 100° C. and stirred for 6 hours. After cooling to 20 to 25° C., the mixture was concentrated under reduced pressure to afford a residue. The residue was dissolved in H₂O, treated with an aqueous saturated solution of NaHCO₃ until pH 10-11 was reached and extracted. The organic layer was dried, filtered and concentrated under reduced pressure to give N-methyl-6-nitro-8-(trifluoromethyl)quinolin-5-amine (1.19 g). The crude product was used in the next step without further purification. LC/MS retention time: 1.053 min, m/z=272 (M+H⁺)

### Step 4: N5-methyl-8-(trifluoromethyl)quinoline-5,6-diamine

To a solution of N-methyl-6-nitro-8-(trifluoromethyl)quinolin-5-amine (7.04 mmol) in CH₃COOCH₂CH₃ (50 ml) at 20 to 25° C. under N₂ atmosphere was added Pd (10% on C, 750 mg, 0.70 mmol). The flask was purged with H₂, and the resulting mixture stirred for 12 to 16 hours. Then, the reaction mixture was filtered und the filtrate was concentrated under reduced pressure to afford N5-methyl-8-(trifluoromethyl)quinoline-5,6-diamine (1.68 g). The crude product was used in the next step without further purification. LC/MS retention time: 0.690 min, m/z=242 (M+H⁺)

### Step 5: 2-(3-ethylsulfonylimidazo[1,2-a]pyridin-2-yl)-1-methyl-5-(trifluoromethyl)imidazo[4,5-f]quinoline (compound C-11)

Compound C-11 was obtained from N5-methyl-8-(trifluoromethyl)quinoline-5,6-diamine by a series of reaction steps as described in Example 1, Steps 3-5. LC-MS retention time: 1,037 min, m/z=461.0 (M+H⁺)

### Example 4: Synthesis of 2-(3-ethylsulfonylimidazo[1,2-a]pyrimidin-2-yl)-6-methoxy-1-methyl-imidazo[4,5-c]quinoline (compound C-17)

### Step-1: synthesis of ethyl imidazo[1,2-a]pyrimidine-2-carboxylate

To a stirred solution of 2-aminopyrimidine (0.010 mol) in acetone (10 mL) was added slowly ethyl 3-bromo-2-oxo-propanoate (0.010 mol) dropwise over a period of 10 min at 20 to 25° C. Subsequently, the reaction mixture was heated to reflux for 2 hours. Then the precipitate was filtered off and the resulting solid was dissolved in a mixture of CH₃CH₂OH:H₂O mixture (10:3) and heated to 65° C. Then, one equivalent of NaHCO₃ was added to the reaction mixture. The reaction mixture was allowed to cool down to 20 to 25° C., and concentrated under reduced pressure. The resulting solid was filtered off to afford ethyl imidazo[1,2-a]pyrimidine-2-carboxylate. (0.9 g). ¹H-NMR (d6-DMSO) 8.99-8.97 (dd, 1H), 8.68-8.67 (dd, 1H), 8.45 (S, 1H), 7.17-7.15 (dd, 1H), 4.33 (q, 2H), 1.33 (t, 3H), LC-MS (M+1)=192

### Step-2: synthesis of ethyl 3-chloroimidazo[1,2-a]pyrimidine-2-carboxylate

Ethyl imidazo[1,2-a]pyrimidine-2-carboxylate (0.005 mol) was dissolved in CHCl₃ (10 mL), upon which Palauchlor (1.31 g) was added at 20 to 25° C. under N₂-atmosphere. The reaction mixture was then stirred at 20 to 25° C. for 12 to 15 hours. Upon completion of the reaction, the reaction mixture was quenched and extracted. The combined organic layers were washed, dried and concentrated under reduced pressure to afford ethyl 3-chloroimidazo[1,2-a]pyrimidine-2-carboxylate. (0.900 g). ¹H-NMR (d6-DMSO) 8.96-8.94 (m, 1H), 8.83-8.81 (m, 1H), 7.37-7.35 (m, 1H), 4.43 (q, 2H), 1.41 (t, 3H). LCMS (M+1)=226

### Step-3: ethyl 3-ethylsulfanylimidazo[1,2-a]pyrimidine-2-carboxylate

To a stirred solution of ethyl 3-chloroimidazo[1,2-a]pyrimidine-2-carboxylate (0.093 mol) in DMF (100 mL) was added sodium ethane thiolate (0.120 mol) in DMF (100 mL) dropwise at 0° C., upon which the resulting reaction mixture was stirred at 0° C. for 2 hours. The reaction was then quenched and the reaction mixture was extracted. The organic layer was washed, dried and concentrated under reduced pressure to afford a crude product. The crude product was purified by flash chromatography to afford ethyl 3-ethylsulfanylimidazo[1,2-a]pyrimidine-2-carboxylate (14 g). ¹H-NMR (d6-DMSO) 9.08-9.07 (m, 1H), 8.77-8.76 (dd, 1H), 7.38-7.30 (dd, 1H), 4.37 (q, 2H), 2.90 (q, 2H), 1.36 (t, 3H), 1.07 (t, 3H) LCMS (M+1)=252

### Step-4: synthesis of ethyl 3-ethylsulfonylimidazo[1,2-a]pyrimidine-2-carboxylate

To a stirred solution of ethyl 3-ethylsulfanylimidazo[1,2-a]pyrimidine-2-carboxylate (0.047 mol) in CH₂Cl₂ (300 mL) was added meta-chloroperoxybenzoic acid (2.3 equivalents) at 0° C. Then the resulting reaction mixture was allowed to warm up to 20 to 25° C. Subsequently, the reaction mixture was stirred 16 hours. The reaction was then quenched with H₂O and a saturated aqueous solution of sodium bisulphite solution was added. Then the reaction mixture was stirred for another 10 minutes upon which an aqueous 10 wt % solution of NaHCO₃ was added. The organic phase was separated off, the aqueous layer was extracted, and the combined organic phases were concentrated under reduced pressure to afford ethyl 3-ethylsulfonylimidazo[1,2-a]pyrimidine-2-carboxylate (12 g). ¹H-NMR (d6-DMSO): 9.32-9.31 (m, 1H), 8.92-8.91 (m, 1H), 7.47-7.45 (m, 1H), 4.41 (q, 2H), 3.67 (q, 2H), 1.38 (t, 3H), 1.26 (t, 3H). LC-MS (M+1)=284

### Step-5: synthesis of 3-ethylsulfonylimidazo[1,2-a]pyrimidine-2-carboxylic acid: hydrochloride

To a stirred solution of ethyl 3-ethylsulfonylimidazo[1,2-a]pyrimidine-2-carboxylate (0.017 mol) in CH₃CH₂OH (75 mL) was added a 2N aqueous solution of KOH (0.070 mol) at 28° C. Then, the resulting reaction mixture was heated at 70° C. for 3 hours. The reaction mixture was then cooled to 20 to 25° C., and concentrated under reduced pressure. The resulting residue was diluted with 40 ml of H₂O and acidified with an aqueous 1N solution of HCl up to pH 3. The mixture was extracted and the combined organic layers were dried under reduced pressure to afford 3-ethylsulfonylimidazo[1,2-a]pyrimidine-2-carboxylic acid; hydrochloride. (3.0 g) ¹H-NMR (d6-DMSO) 9.57-9.55 (m, 1H), 8.92-8.91 (m, 1H), 7.48-7.46 (m, 1H), 3.65 (q, 2H), 1.26 (t, 3H). LC-MS (M+1)=256

### Step-7: synthesis of 2-(3-ethylsulfonylimidazo[1,2-a]pyrimidin-2-yl)-6-methoxy-1-methyl-imidazo[4,5-c]quinoline

Compounds 3-ethylsulfonylimidazo[1,2-a]pyrimidine-2-carboxylic acid; hydrochloride and N4-methyl-8-(trifluoromethoxy)quinoline-3,4-diamine were converted to afford 2-(3-ethylsulfonylimidazo[1,2-a]pyrimidin-2-yl)-6-methoxy-1-methyl-imidazo[4,5-c]quinoline in a series of reaction steps in analogy to Example 1, Steps 3 and 4. LC-MS (M+1)=476.9, retention time: 0,866 With appropriate modification of the starting materials or intermediates thereof, the procedures as described in the preparation examples above were used to obtain further compounds of formula I. The compounds obtained in this manner are listed in the below Table C, together with physical data.

TABLE C
List of compounds C-1 to C-20 with physical characterization data

| Com- | | | |
|---|---|---|---|
| pound | | HPLC/MS | Rt |
| no. | Structure | (M + 1) [g/mol] | [min] |
| C-1 | 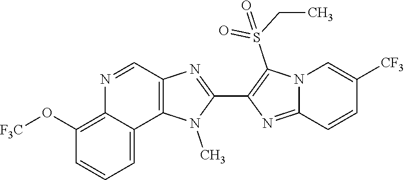 [SMILES: CCS(=O)(=O)c1c(-c2nc3cnc4c(OC(F)(F)F)cccc4c3n2C)nc2ccc(C(F)(F)F)cn12] | 544 | 1.165 |
| C-2 | 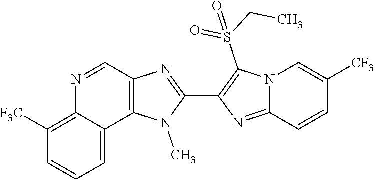 [SMILES: CCS(=O)(=O)c1c(-c2nc3cnc4c(C(F)(F)F)cccc4c3n2C)nc2ccc(C(F)(F)F)cn12] | 528.1 | 1.222 |
| C-3 | 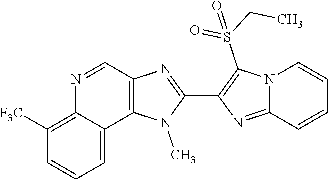 [SMILES: CCS(=O)(=O)c1c(-c2nc3cnc4c(C(F)(F)F)cccc4c3n2C)nc2ccccn12] | 459.9 | 1.022 |
| C-4 | 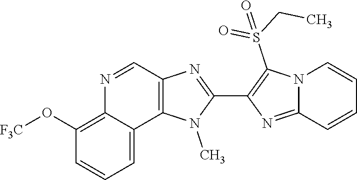 [SMILES: CCS(=O)(=O)c1c(-c2nc3cnc4c(OC(F)(F)F)cccc4c3n2C)nc2ccccn12] | 476.0 | 0.968 |
| C-5 | 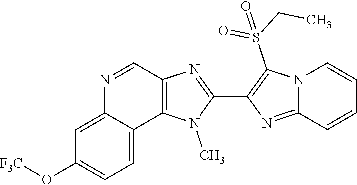 [SMILES: CCS(=O)(=O)c1c(-c2nc3cnc4cc(OC(F)(F)F)ccc4c3n2C)nc2ccccn12] | 494.0 | 0.852 |
| C-6 | 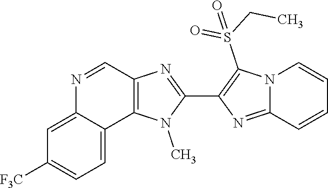 [SMILES: CCS(=O)(=O)c1c(-c2nc3cnc4cc(C(F)(F)F)ccc4c3n2C)nc2ccccn12] | 460.0 | 0.938 |
| C-7 | 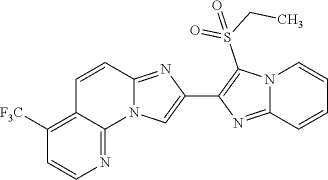 [SMILES: CCS(=O)(=O)c1c(-c2cn3c(ccc4c(C(F)(F)F)ccnc43)n2)nc2ccccn12] | 446.0 | 1.008 |
| C-8 | 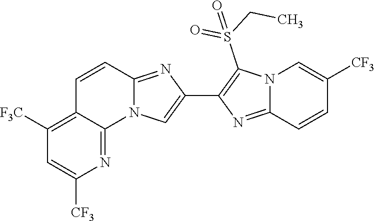 [SMILES: CCS(=O)(=O)c1c(-c2cn3c(ccc4c(C(F)(F)F)cc(C(F)(F)F)nc43)n2)nc2ccc(C(F)(F)F)cn12] | 582.0 | 1,351 |
| C-9 | 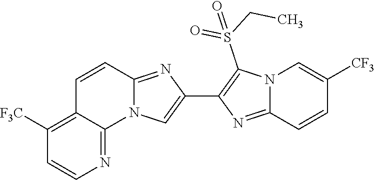 [SMILES: CCS(=O)(=O)c1c(-c2cn3c(ccc4c(C(F)(F)F)ccnc43)n2)nc2ccc(C(F)(F)F)cn12] | 514.0 | 1.217 |
| C-10 | 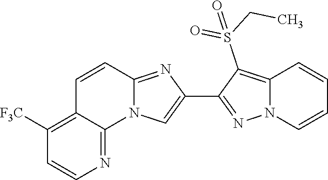 [SMILES: CCS(=O)(=O)c1c(-c2cn3c(ccc4c(C(F)(F)F)ccnc43)n2)nn2ccccc12] | 446.0 | 1.068 |
| C-11 | 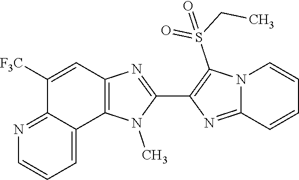 [SMILES: CCS(=O)(=O)c1c(-c2nc3cc(C(F)(F)F)c4ncccc4c3n2C)nc2ccccn12] | 461.0 | 1.037 |
| C-12 | 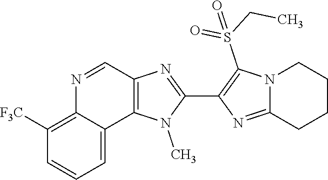 [SMILES: CCS(=O)(=O)c1c(-c2nc3cnc4c(C(F)(F)F)cccc4c3n2C)nc2n1CCCC2] | 464.3 | 1.008 |
| C-13 | 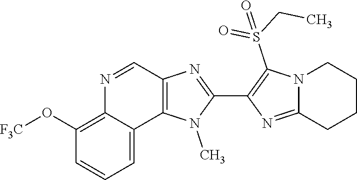 [SMILES: CCS(=O)(=O)c1c(-c2nc3cnc4c(OC(F)(F)F)cccc4c3n2C)nc2n1CCCC2] | 484.3 | 0.89 |
| C-14 | 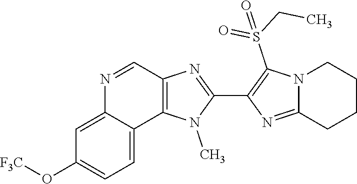 [SMILES: CCS(=O)(=O)c1c(-c2nc3cnc4cc(OC(F)(F)F)ccc4c3n2C)nc2n1CCCC2] | 480.3 | 0.877 |
| C-15 | 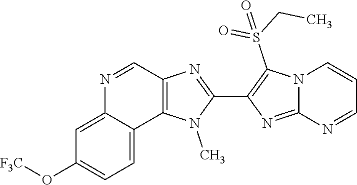 [SMILES: CCS(=O)(=O)c1c(-c2nc3cnc4cc(OC(F)(F)F)ccc4c3n2C)nc2ncccn12] | 477.3 | 0.85 |
| C-16 | 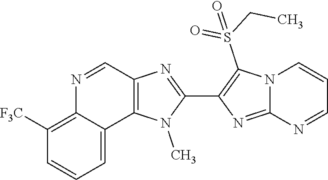 [SMILES: CCS(=O)(=O)c1c(-c2nc3cnc4c(C(F)(F)F)cccc4c3n2C)nc2ncccn12] | 461.3 | 0.86 |
| C-17 | 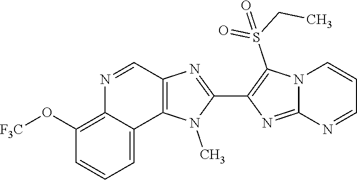 [SMILES: CCS(=O)(=O)c1c(-c2nc3cnc4c(OC(F)(F)F)cccc4c3n2C)nc2ncccn12] | 476.9 | 0.866 |
| C-18 | 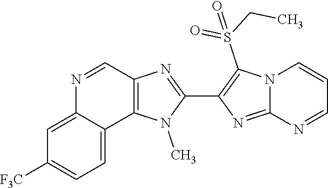 [SMILES: CCS(=O)(=O)c1c(-c2nc3cnc4cc(C(F)(F)F)ccc4c3n2C)nc2ncccn12] | 460.9 | 0.87 |
| C-19 | 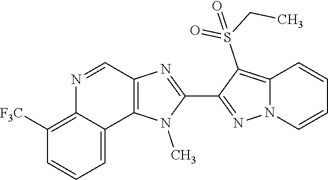 [SMILES: CCS(=O)(=O)c1c(-c2nc3cnc4c(C(F)(F)F)cccc4c3n2C)nn2ccccc12] | 459.9 | 1.091 |
| C-20 | 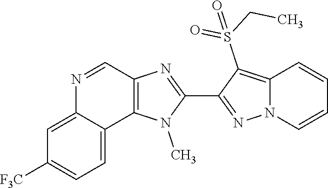 [SMILES: CCS(=O)(=O)c1c(-c2nc3cnc4cc(C(F)(F)F)ccc4c3n2C)nn2ccccc12] | 460.2 | 0.956 |
| C-21 | 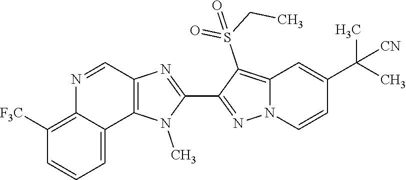 [SMILES: CCS(=O)(=O)c1c(-c2nc3cnc4c(C(F)(F)F)cccc4c3n2C)nn2ccc(C(C)(C)C#N)cc12] | 527.0 | 1.163 |
| C-22 | 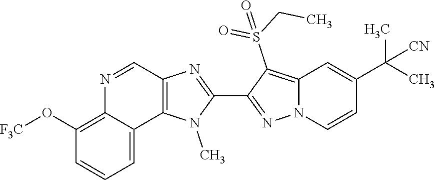 [SMILES: CCS(=O)(=O)c1c(-c2nc3cnc4c(OC(F)(F)F)cccc4c3n2C)nn2ccc(C(C)(C)C#N)cc12] | 543.0 | 1.067 |
| C-23 | 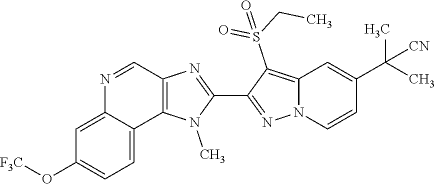 [SMILES: CCS(=O)(=O)c1c(-c2nc3cnc4cc(OC(F)(F)F)ccc4c3n2C)nn2ccc(C(C)(C)C#N)cc12] | 543.0 | 1.081 |
| C-24 | 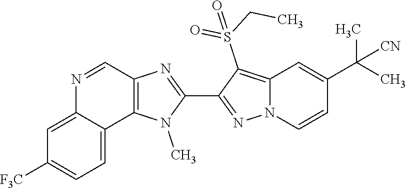 [SMILES: CCS(=O)(=O)c1c(-c2nc3cnc4cc(C(F)(F)F)ccc4c3n2C)nn2ccc(C(C)(C)C#N)cc12] | 527.0 | 1.058 |
| C-25 | 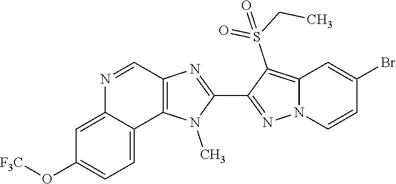 [SMILES: CCS(=O)(=O)c1c(-c2nc3cnc4cc(OC(F)(F)F)ccc4c3n2C)nn2ccc(Br)cc12] | 554.2 | 0.99 |
| C-26 | 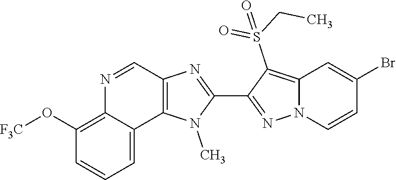 [SMILES: CCS(=O)(=O)c1c(-c2nc3cnc4c(OC(F)(F)F)cccc4c3n2C)nn2ccc(Br)cc12] | 553.8 | 1.106 |
| C-27 | 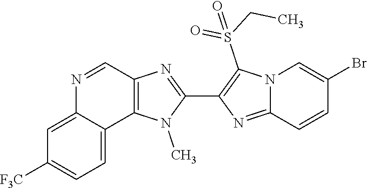 [SMILES: CCS(=O)(=O)c1c(-c2nc3cnc4cc(C(F)(F)F)ccc4c3n2C)nc2ccc(Br)cn12] | 538.2 | 1.035 |
| C-28 | 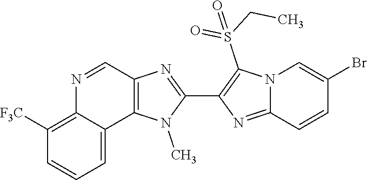 [SMILES: CCS(=O)(=O)c1c(-c2nc3cnc4c(C(F)(F)F)cccc4c3n2C)nc2ccc(Br)cn12] | 539.8 | 1.174 |
| C-29 | 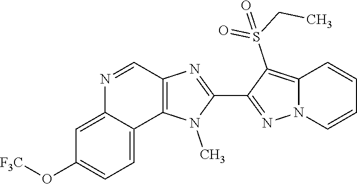 [SMILES: CCS(=O)(=O)c1c(-c2nc3cnc4cc(OC(F)(F)F)ccc4c3n2C)nn2ccccc12] | 476.2 | 0.93 |
| C-30 | 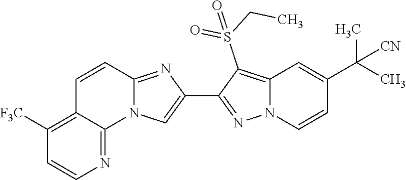 [SMILES: CCS(=O)(=O)c1c(-c2cn3c(ccc4c(C(F)(F)F)ccnc43)n2)nn2ccc(C(C)(C)C#N)cc12] | 512.9 | 1.172 |

### B. BIOLOGICAL EXAMPLES

The activity of the compounds of formula (I) of the present invention could be demonstrated and evaluated in biological tests described in the following. If not otherwise specified, the test solutions are prepared as follows: The active compound is dissolved at the desired concentration in a mixture of 1:1 (vol:vol) distilled water:acetone. The test solution is prepared at the day of use. Test solutions are prepared in general at concentrations of 2500 ppm, 1000 ppm, 800 ppm, 500 ppm, 300 ppm, 100 ppm and 30 ppm (wt/vol).

Boll Weevil (Anthonomus grandis)

For evaluating control of boll weevil (Anthonomus grandis) the test unit consisted of 96-well-microtiter plates containing an insect diet and 5-10 A. grandis eggs. The compounds were formula ted using a solution containing 75% v/v water and 25% v/v DMSO. Different concentrations of formulated compounds were sprayed onto the insect diet at 5 μl, using a custom built micro atomizer, at two replications. After application, microtiter plates were incubated at about 25±1° C. and about 75±5% relative humidity for 5 days. Egg and larval mortality was then visually assessed. In this test, compounds C-1, C-2, C-3, C-4, C-5, C-6, C-7 at 2500 ppm showed over 75% mortality in comparison with untreated controls. In this test, compounds C-8, C-9, C-10, C-11, C-12, C-13, C-19, C-20, C-22, C-23, C-27, C-28, and C-29 at 800 ppm showed over 75% mortality in comparison with untreated controls.

Tobacco Budworm (Heliothis virescens)

For evaluating control of tobacco budworm (Heliothis virescens) the test unit consisted of 96-well-microtiter plates containing an insect diet and 15-25 H. virescens eggs. The compounds were formulated using a solution containing 75% v/v water and 25% v/v DMSO. Different concentrations of formulated compounds were sprayed onto the insect diet at 10 μl, using a custom built micro atomizer, at two replications. After application, microtiter plates were incubated at about 28±1° C. and about 80±5% relative humidity for 5 days. Egg and larval mortality was then visually assessed. In this test, compounds C-1, C-2, C-3, C-4, C-5, C-6, C-7 at 2500 ppm showed over 75% mortality in comparison with untreated controls. In this test, compounds C-8, C-9, C-11, C-12, C-13, C-14, C-18, C-19, C-20, C-22, C-23, C-27, C-28, C-29 at 800 ppm showed over 75% mortality in comparison with untreated controls.

Green Peach Aphid (Myzus persicae) For evaluating control of green peach aphid (Myzus persicae) through systemic means the test unit consisted of 96-well-microtiter plates containing liquid artificial diet under an artificial membrane. The compounds were formulated using a solution containing 75% v/v water and 25% v/v DMSO. Different concentrations of formulated compounds were pipetted into the aphid diet, using a custom built pipetter, at two replications. After application, 5-8 adult aphids were placed on the artificial membrane inside the microtiter plate wells. The aphids were then allowed to suck on the treated aphid diet and incubated at about 23±1° C. and about 50±5% relative humidity for 3 days. Aphid mortality and fecundity was then visually assessed. In this test, compounds C-1, C-2, C-3, C-4, C-5, C-7 at 2500 ppm showed over 75% mortality in comparison with untreated controls. In this test, compounds C-9, C-10, C-11, C-12, C-13, C-14, C-15, C-16, C-17, C-19, C-22, C-28, C-29 at 800 ppm showed over 75% mortality in comparison with untreated controls.

Greenhouse Whitefly (Trialeurodes vaporariorum)

For evaluating control of Greenhouse Whitefly (Trialeurodes vaporariorum) the test unit consisted of 96-well-microtiter plates containing a leaf disk of egg plant leaf disk with white fly eggs. The compounds or mixtures were formulated using a solution containing 75% water and 25% DMSO. Different concentrations of formulated were sprayed onto the insect diet at 2.5 μl, using a custom built micro atomizer, at two replications. After application, microtiter plates were incubated at 23±1° C., 65±5% RH for 6 days. Mortality of hatched crawlers was then visually assessed. In this test, compound C-13 at 800 ppm showed over 75% mortality in comparison with untreated controls.

Yellow Fever Mosquito (Aedes aegypti)

For evaluating control of yellow fever mosquito (Aedes aegypti) the test unit consisted of 96-well-microtiter plates containing 200 μl of tap water per well and 5-15 freshly hatched A. aegypti larvae. The active compounds were formulated using a solution containing 75% (v/v) water and 25% (v/v) DMSO. Different concentrations of formulated compounds or mixtures were sprayed onto the insect diet at 2.5 μl, using a custom built micro atomizer, at two replications. After application, microtiter plates were incubated at 28±1° C., 80±5% RH for 2 days. Larval mortality was then visually assessed. In this test, compounds C-1, C-2, C-3, C-4, C-5, C-7, C-9, C-12, C-19, C-27, C-28, C-29 at 800 ppm showed at least 75% mortality in comparison with untreated controls.

Vetch Aphid (Megoura viciae)

For evaluating control of vetch aphid (Megoura viciae) through contact or systemic means the test unit consisted of 24-well-microtiter plates containing broad bean leaf disks.

The compounds were formulated using a solution containing 75% v/v water and 25% v/v DMSO. Different concentrations of formulated compounds were sprayed onto the leaf disks at 2.5 μl, using a custom built micro atomizer, at two replications. After application, the leaf disks were air-dried and 5-8 adult aphids placed on the leaf disks inside the microtiter plate wells. The aphids were then allowed to suck on the treated leaf disks and incubated at about 23±1° C. and about 50±5% relative humidity for 5 days. Aphid mortality and fecundity was then visually assessed. In this test, compounds C-1, C-2, C-3, C-4, at 2500 ppm showed over 75% mortality in comparison with untreated controls.

1. A compound of formula (I), or an agrochemically or veterinarily acceptable salt, stereoisomer, tautomer, or N-oxide thereof 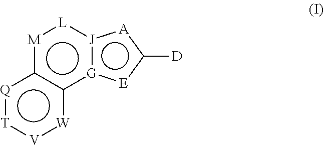
wherein the variables in formula (I) have the following meaning,
A is CH, N, or NH;
E is N, O, S, NR^{E}, or CR^{E};
G, J are independently C or N;
L is N or CR^{L};
M is N or CR^{M};
Q is N or CR^{Q};
T is N or CR^{T};
V is N or CR^{V};
W is N or CR^{W};
R^{E}, R^{L}, R^{M}, R^{Q}, R^{T}, R^{V}, and R^{W} are independently selected from H, halogen, N₃, CN, NO₂, SCN, SF₅, C₁-C₆-alkyl, C₁-C₆-alkoxy, C₂-C₆-alkenyl, tri-C₁-C₆-alkylsilyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxyx-C₁-C₄-alkyl, which groups are unsubstituted or substituted with halogen;
C(═O)OR¹, NR²R³, C₁-C₆-alkylen-NR²R³, O—C₁-C₆-alkylen-NR²R³, C₁-C₆-alkylen-CN, NH—C₁-C₆-alkylen-NR²R³, C(═O)NR²R³, C(═O)R⁴, SO₂NR²R³, S(═O)_{q}R⁵, OR⁶, SR⁶, phenyl, and benzyl, wherein the phenyl ring g is unsubstituted or substituted with one or more, same or different substituents R¹¹;
R¹ is H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, or C₃-C₆-cycloalkoxy-C₁-C₄-alkyl, which groups are unsubstituted or substituted with halogen;
C₁-C₆-alkylen-NR²R³, C₁-C₆-alkylen-CN, or
phenyl or benzyl, wherein the phenyl ring is unsubstituted, or substituted with one or more, same or different substituents R¹¹;
R¹¹ is selected from halogen, N₃, OH, CN, NO₂, SCN, SF₅,
C₁-C₆-alkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, which groups are unsubstituted or substituted with halogen;
R² is H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted, or substituted with one or more, same or different substituent selected from halogen, CN and HO;
C(═O)R²¹, C(═O)OR²¹, C(═O)NR²¹, C₁-C₆-alkylen-CN, or phenyl or benzyl, wherein the phenyl ring is unsubstituted or substituted with one or more, same or different substituents R¹¹;
R²¹ is H, C₁-C₆-alkyl, C₁-C₆-haloalkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄ alkyl, phenyl, or a saturated, partially-, or fully unsaturated 5- or 6-membered heterocycle, wherein the cyclic moieties are unsubstituted or substituted with one or more, same or different substituents R¹¹;
R³ is H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen;
C₁-C₆-alkylen-CN, or phenyl or benzyl, wherein the phenyl ring is unsubstituted or substituted with one or more, same or different substituents R¹; or
NR²R³ may also form an N-bound, saturated 3- to 8-membered heterocycle, which in addition to the nitrogen atom may have 1 or 2 further heteroatoms or heteroatom moieties selected from 0, S(═O)_{q}, NH, and N—C₁-C₆-alkyl, and wherein the N-bound heterocycle is unsubstituted or substituted with one or more, same or different substituents selected from halogen, C₁-C₄-alkyl, C₁-C₄-haloalkyl, C₁-C₄-alkoxy and C₁-C₄-haloalkoxy;
R⁴ is H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, or C₃-C₆-cycloalkoxy-C₁-C₄-alkyl, which groups are unsubstituted or substituted with one or more, same of different substituents selected from halogen, CN, and OH;
phenyl or benzyl, wherein the phenyl ring unsubstituted, or substituted with one or more, same or different substituents R¹¹;
R⁵ is C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, or C₃-C₆-cycloalkoxy-C₁-C₄-alkyl, which groups are unsubstituted or substituted with halogen;
C₁-C₆-alkylen-NR²R³, C₁-C₆-alkylen-CN, phenyl or benzyl, wherein the phenyl ring is unsubstituted, or substituted with one or more, same or different substituents R¹¹;
R⁶ is phenyl, which is unsubstituted or substituted with one or more, same or different substituents R¹¹;
D is a moiety of formula 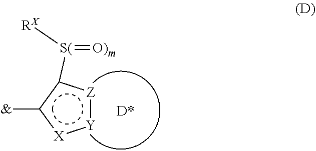
wherein the “&”-symbol signifies the connection to the remainder of formula (I), wherein the dotted circle in the 5-membered ring means that the 5-membered ring may be saturated, partially unsaturated, or fully unsaturated;
R^{X} is C₁-C₆-alkyl, C₃-C₆-cycloalkyl, C₃-C₆-cycloalkyl-C₁-C₄-alkyl, which are unsubstituted or substituted with halogen; or
phenyl or benzyl, wherein the phenyl ring is unsubstituted or substituted with one or more, same or different substituents R¹¹;
X is N, S, O, CR⁷, or NR⁸;
Y and Z are independently C or N, wherein at least one of the variables selected from Y and Z is C;
D* is a 5- or 6-membered saturated, partially unsaturated, or fully unsaturated carbo- or heterocycle, which carbo- or heterocycle includes the atoms Y and Z as ring members and is unsubstituted or substituted with one or more, same or different substituents R⁹, and wherein said heterocycle comprises 0, 1, 2, or 3, same or different heteroatoms O, N, or S in addition to those that may be present as ring members Y and Z;
R⁷ is H, halogen, OH, CN, NC, NO₂, N₃, SCN, NCS, NCO, SF₅,
C₁-C₆-alkyl, C₃-C₆-cycloalkyl, C₂-C₆-alkenyl, C₃-C₆-cycloalkenyl, C₂-C₆-alkynyl, which groups are unsubstituted, or substituted with one or more, same or different substituents R^{G1};
a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents R^{H1}, and wherein said N- and S-atoms are independently oxidized, or non-oxidized;
phenyl, which is unsubstituted, or substituted with one or more, same or different substituents R^{J1};
OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═S)SR^{K1}, OS(═O)_{q}R^{K1}, OS(═O)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)_{q}R^{V1}, SC(═O)SR^{K1}, SC(═O)NR^{L1}R^{M1}, S(═O)_{q}NR^{L1}R^{M1}, C(═O)R^{P1}, C(═S)R^{P1}, C(═O)NR^{L1}R^{M1}, C(═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, Si(R^{S1})₂R^{T1};
R⁸ is H, CN, C₁-C₆-alkyl, C₃-C₆-cycloalkyl, C₂-C₆-alkenyl, C₃-C₆-cycloalkenyl, C₂-C₆-alkynyl, which groups are unsubstituted or substituted with one or more, same or different substituents R^{G1};
a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents R^{H1}, and wherein said N- and S-atoms are independently oxidized, or non-oxidized;
phenyl, which is unsubstituted, or substituted with one or more, same or different substituents R^{J1};
OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═S)SR^{K1}, OS(═O)_{q}R^{K1}, OS(═O)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)_{q}R^{V1}, SC(═O)SR^{K1}, SC(═O)NR^{L1}R^{M1}, S(═O)_{q}NR^{L1}R^{M1}, C(═O)R^{P1}, C(═S)R^{P1}, C(═O)NR^{L1}R^{M1}, C(═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, or Si(R^{S1})₂R^{T1};
each R⁹ is independently H, halogen, OH, CN, NC, NO₂, N₃, SCN, NCS, NCO, SF₅,
C₁-C₆-alkyl, C₃-C₆-cycloalkyl, C₂-C₆-alkenyl, C₃-C₆-cycloalkenyl, or C₂-C₆-alkynyl, C₃-C₆-cycloalkyl-C₁-C₃-alkyl, which groups are unsubstituted, or substituted with one or more, same or different substituents R^{G1};
a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents R^{H1}, and wherein said N- and S-atoms are independently oxidized, or non-oxidized;
phenyl, which is unsubstituted, or substituted with one or more, same or different substituents R^{J1};
OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═S)SR^{K1}, OS(═O)_{q}R^{K1}, OS(═O)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)_{q}R^{V1}, SC(═O)SR^{K1}, SC(═O)NR^{L1}R^{M1}, S(═O)_{q}NR^{L1}R^{M1}, C(═O)R^{P1}, C(═S)R^{P1}, C(═O)NR^{L1}R^{M1}, C(═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, or Si(R^{S1})₂R^{T1};
or two substituents R^{G1} form, together with the ring members of ring D to which they are bound, a 5- or 6-membered saturated, partially unsaturated, or fully unsaturated carbo- or heterocycle, which carbo- or heterocycle is unsubstituted, or substituted with one or more, same or different substituents R^{J1}, and wherein said heterocycle comprises one or more, same or different heteroatoms O, N, or S;
each R^{G1} is independently halogen, OH, CN, NC, NO₂, C₁-C₆-alkyl, C₃-C₆-cycloalkyl, C₃-C₆-cycloalkenyl, which groups are unsubstituted or substituted with one or more, same or different substituents selected from halogen, OH, CN, C₁-C₃-alkoxy, C₁-C₃-haloalkoxy, and C₁-C₃-alkyl-carbonyl;
a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents selected from halogen, OH, CN, C₁-C₃-alkoxy, C₁-C₃-haloalkoxy, and C₁-C₃-alkyl-carbonyl, and wherein said N- and S-atoms are independently oxidized, or non-oxidized;
phenyl, which is unsubstituted or substituted with one or more, same or different substituents selected from halogen, OH, CN, NO₂, C₁-C₃-alkyl, C₁-C₃-haloalkyl, C₁-C₃-alkoxy, C₁-C₃-haloalkoxy, and C₁-C₃-alkyl-carbonyl; OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═S)SR^{K1}, OS(═O)_{q}R^{K1}, OS(═O)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)_{q}R^{V1}, SC(═O)SR^{K1}, SC(═O)NR^{L1}R^{M1}, S(═O)_{q}NR^{L1}R^{M1}, C(═O)R^{P1}, C(═S)R^{P1}, C(═O)NR^{L1}R^{M1}, C(═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, Si(R^{S1})₂R^{T1};
each R^{H1} is independently halogen, CN, NC, NO₂, SCN, NCS, NCO,
C₁-C₆-alkyl, C₃-C₆-cycloalkyl, C₃-C₆-cycloalkenyl, which groups are unsubstituted, or substituted with one or more, same or different substituents selected from halogen, OH, CN, C₁-C₁₀-alkoxy, C₁-C₃-haloalkoxy, and C₁-C₃-alkyl-carbonyl;
phenyl, which is unsubstituted, or substituted with one or more, same or different substituents selected from halogen, OH, CN, NO₂, C₁-C₃-alkyl, C₁-C₃-haloalkyl, OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═S)SR^{K1}, OS(═O)_{q}R^{K1}, OS(═O)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)_{q}R^{V1}, SC(═O)SR^{K1}, SC(═O)NR^{L1}R^{M1}, S(═O)_{q}NR^{L1}R^{M1}, C(═O)R^{P1}, C(═S)R^{P1}, C(═O)NR^{L1}R^{M1}, C(═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, Si(R^{S1})₂R^{T1}; or
two geminal substituents R^{H1} form together with the atom to which they are bound a group ═O, ═S, or ═NR^{L};
each R^{J1} is independently halogen, CN, NC, NO₂, SCN, NCS, NCO, C₁-C₆-alkyl, C₃-C₆-cycloalkyl, C₃-C₆-cycloalkenyl, which groups are unsubstituted, or substituted with one or more, same or different substituents selected from halogen, OH, CN, C₁-C₁₀-alkoxy, C₁-C₃-haloalkoxy, and C₁-C₃-alkyl-carbonyl;
phenyl, which is unsubstituted, or substituted with one or more, same or different substituents selected from halogen, OH, CN, NO₂, C₁-C₃-alkyl, C₁-C₃-haloalkyl, OR^{K1}, SR^{K1}, OC(═O)R^{K1}, OC(═O)OR^{K1}, OC(═O)NR^{L1}R^{M1}, OC(═O)SR^{K1}, OC(═S)NR^{L1}R^{M1}, OC(═S)SR^{K1}, OS(═O)_{q}R^{K1}, OS(═O)_{q}NR^{L1}R^{M1}, ONR^{L1}R^{M1}, ON═CR^{N1}R^{O1}, NR^{L1}R^{M1}, NOR^{K1}, ONR^{L1}R^{M1}, N═CR^{N1}R^{O1}, NNR^{L1}, N(R^{L1})C(═O)R^{K1}, N(R^{L1})C(═O)OR^{K1}, S(═O)_{q}R^{V1}, SC(═O)SR^{K1}, SC(═O)NR^{L1}R^{M1}, S(═O)_{q}NR^{L1}R^{M1}, C(═O)R^{P1}, C(═S)R^{P1}, C(═O)NR^{L1}R^{M1}, C(═O)OR^{K1}, C(═S)NR^{L1}R^{M1}, C(═S)OR^{K1}, C(═S)SR^{K1}, C(═NR^{L1})R^{M1}, C(═NR^{L1})NR^{M1}R^{R1}, Si(R^{S1})₂R^{T1};
each R^{K1} is independently H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with one or more, same or different substituents selected from halogen, CN, NR^{M1}R^{N1}; C(═O)NR^{M1}R^{N1}, C(═O)R^{T1}; or
phenyl or benzyl, wherein the phenyl ring is unsubstituted or substituted with one or more, same or different substituents R^{X1};
each R^{L1} is independently H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen;
C₁-C₆-alkylen-CN;
phenyl and benzyl, which groups are unsubstituted or substituted with one or more, same or different substituents R^{X1};
each R^{M1}, R^{R1} is independently H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen;
C₁-C₆-alkylen-CN; or
phenyl or benzyl, wherein the phenyl ring is unsubstituted or substituted with one or more, same or different substituents R^{X1};
each moiety NR^{M1}R^{R1} or NR^{L1}R^{M1} may also form an N-bound, saturated 5- to 8-membered heterocycle, which in addition to the nitrogen atom may have 1 or 2 further heteroatoms or heteroatom moieties selected from O, S(═O)_{q}, and N—R′, wherein R′ is H or C₁-C₆-alkyl and wherein the N-bound heterocycle is unsubstituted or substituted with one or more, same or different substituents selected from halogen, C₁-C₄-alkyl, C₁-C₄-haloalkyl, C₁-C₄-alkoxy and C₁-C₄-haloalkoxy;
each R^{N1} is independently H, halogen, CN, NO₂, SCN, C₁-C₁₀-alkyl, C₃-C₅-cycloalkyl, C₂-C₆-alkenyl, C₃-C₆-cycloalkenyl, C₂-C₆-alkynyl, which groups are unsubstituted, or substituted with one or more, same or different substituents selected from halogen, C₁-C₆-alkyl, C₁-C₆-alkoxy, C₁-C₆-haloalkyl, and C₁-C₆-haloalkoxy;
a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more, same or different substituents selected from halogen, C₁-C₃-alkyl, C₁-C₃-alkoxy, C₁-C₃-haloalkyl, and C₁-C₃-haloalkoxy, and wherein said N- and S-atoms are independently oxidized, or non-oxidized;
phenyl, which is unsubstituted, or substituted with one or more, same or different substituents selected from halogen, C₁-C₃-alkyl, C₁-C₃-alkoxy, C₁-C₃-haloalkyl, and C₁-C₃-haloalkoxy;
each R^{O1} is independently H, C₁-C₄-alkyl, C₁-C₆-cycloalkyl, C₁-C₂-alkoxy-C₁-C₂-alkyl, phenyl, or benzyl;
each R^{P1} is independently H, C₁-C₆-alkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which groups are unsubstituted or substituted with halogen;
phenyl or benzyl, wherein the phenyl ring is unsubstituted or substituted with one or more, same or different substituents R^{X1};
each R^{S1}, R^{T1} is independently H, C₁-C₆-alkyl, C₁-C₆-haloalkyl, C₁-C₆-alkoxy, C₁-C₄-alkoxy-C₁-C₄-alkyl, C₃-C₆-cycloalkyl, C₃-C₆-halocycloalkyl, C₁-C₄-haloalkoxy-C₁-C₄-alkyl, or phenyl;
each R^{V1} is independently C₁-C₆-alkyl, C₃-C₆-cycloalkyl, C₃-C₆-cycloalkyl-C₁-C₄-alkyl, which are unsubstituted or substituted with halogen; or
phenyl or benzyl, wherein the phenyl ring is unsubstituted or substituted with R^{X1};
each R^{X1} is independently halogen, N₃, OH, CN, NO₂, SCN, SF₅, C₁-C₆-alkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, which groups are unsubstituted or substituted with halogen;
the index m is 0, 1, or 2;
the index q is 0, 1, or 2.

2. The compound of formula (I) according to claim 1, wherein A is N.

3. The compound of formula (I) according to claim 1, wherein formula (I) is selected from formulae (I-A), (I-C), and (I-D).

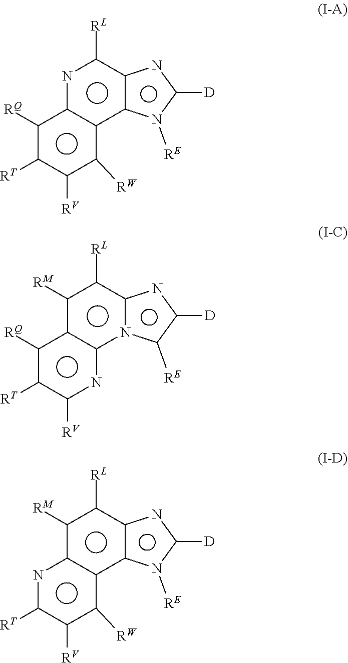

4. The compound of formula (I) according to claim 1, wherein
R^{L}, R^{M}, R^{Q}, R^{T}, R^{V}, and R^{W} independently are selected from H, halogen,
C₁-C₆-alkyl, C₁-C₆-alkoxy, C₂-C₆-alkenyl, C₂-C₆-alkynyl, C₃-C₆-cycloalkyl, C₃-C₆-cycloalkoxy, and C₁-C₆-alkyl-S(═O)_{q}, which groups are unsubstituted or substituted with halogen.

5. The compound of formula (I) according to claim 1, wherein D is selected from the formulae D1, D3, D8, and D50, 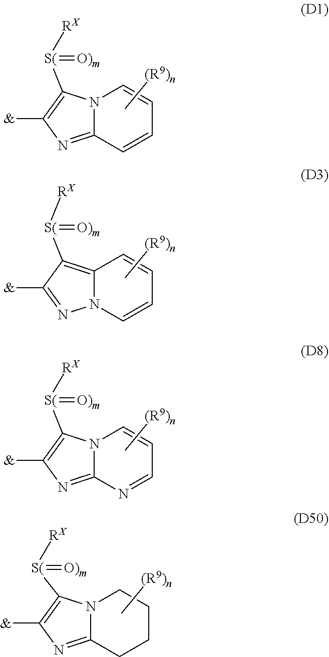
wherein n is 0, 1, 2, 3, or 4.

6. The compound of formula (I) according to claim 1, wherein R^{X} is C₁-C₄-alkyl, which is unsubstituted or substituted with halogen.

7. The compound of formula (I) according to claim 1, wherein R⁹ is independently selected from H, halogen, OH, CN, C₁-C₃-alkyl, C₁-C₃-alkoxy, C₂-C₃-alkenyl, C₂-C₃-alkynyl, and C₃-C₆-cycloalkyl, which groups are unsubstituted or substituted with CN or halogen.

8. (canceled)

9. A pesticidal mixture comprising a compound of formula (I) as defined in claim 1, and another agrochemically active ingredient.

10. An agrochemical or veterinary composition comprising a compound of formula (I) as defined in claim 1 and a liquid or solid carrier.

11. A method for controlling invertebrate pests, infestation, or infection by invertebrate pests, comprising contacting the pests, their food supply, habitat, breeding grounds or their locus with a compound of formula (I) as defined in claim 1 in a pesticidally effective amount.

12. A method for protecting growing plants from attack or infestation by invertebrate pests, comprising contacting a plant, or soil or water in which the plant is growing, with a pesticidally effective amount of at least one compound of the formula (I), according to claim 1.

13. A seed comprising a compound of formula (I) as defined in claim 1 in an amount of from 0.1 g to 10 kg per 100 kg of seeds.

14. A method for treating, or protecting an animal against infestation or infection by a parasite, or controlling, or preventing infestations or infections of animals by a parasite, comprising administering or applying orally, topically, or parenterally to the animal a compound of the general formula (I) as defined in claim 1.

15. The pesticidal mixture of claim 9 wherein the agrochemically active ingredient is an insecticide, fungicide, or mixture thereof.
